SEQUENCE LISTING (23 sequences)

SEQ ID NO: 1 - protein, 585 aa, Artificial Sequence
  Description of Artificial Sequence/note = synthetic construct
MAAVELEWIPETLYNTAISAVVDNYIRSRRDIRSLPENIQFDVYYKLYQQGRLCQLGSEFCELEVFAKVLRALDKRHLLHHCFQALMDHGVKVASVLAYSFSRRCSYIAESDAAVKEKAIQVGFVLGGFLSDAGWYSDAEKVFLSCLQLCTLHDEMLHWFRAVECCVRLLHVRNGNCKYHLGEETFKLAQTYMDKLSKHGQQANKAALYGELCALLFAKSHYDEAYKWCIEAMKEITAGLPVKVVVDVLRQASKACVVKREFKKAEQLIKHAVYLARDHFGSKHPKYSDTLLDYGFYLLNVDNICQSVAIYQAALDIRQSVFGGKNIHVATAHEDLAYSSYVHQYSSGKFDNALFHAERAIGIITHILPEDHLLLASSKRVKALILEEIAIDCHNKETEQRLLQEAHDLHLSSLQLAKKAFGEFNVQTAKHYGNLGRLYQSMRKFKEAEEMHIKAIQIKEQLLGQEDYEVALSVGHLASLYNYDMNQYENAEKLYLRSIAIGKKLFGEGYSGLEYDYRGLIKLYNSIGNYEKVFEYHNVLSNWNRLRDRQYSVTDALEDVSTSPQSTEEVVQSFLISQNVEGPSC

SEQ ID NO: 2 - DNA, 1758 bp, Artificial Sequence
  Description of Artificial Sequence/note = synthetic construct
atggcggccgtggaactagagtggatcccagagactctctataacaccgccatctccgctgtcgtggacaactacatccgctcccgccgagacatccgctccttgcccgagaacatccagtttgatgtttactacaagctttaccaacagggacgcttatgtcaactgggcagtgaattttgtgaattggaagtttttgctaaagtactgagagctttggataaaagacatttgcttcatcattgttttcaggctttgatggatcatggtgttaaagttgcttcagtcttggcctactcattcagtaggcggtgctcttatatagcagaatcagatgctgcagtaaaggaaaaagccattcaggttggctttgttttaggtggctttctttcagatgcaggctggtacagtgatgctgagaaagtttttctgtcctgccttcagttgtgtactctacacgatgagatgcttcattggtttcgtgcagtagaatgttgtgtgaggttgcttcatgtgcgaaatggaaactgcaaatatcatttgggtgaagaaacatttaaattagctcagacatatatggataaactatcaaaacatggccagcaagcaaataaagctgcactctatggagaactgtgtgcactcctatttgcaaaaagtcactatgatgaggcatacaaatggtgcatcgaggcaatgaaagaaattacagcaggcttaccagtgaaagttgtggtggatgtcttaagacaagcttctaaggcttgtgtagtaaaacgtgaatttaagaaggcagaacagttaattaaacatgcagtgtatttggcacgggatcattttggatccaaacacccaaaatattctgatacactgctagattatgggttctacttactcaatgtagataatatctgtcagtctgttgcaatttatcaggcagcccttgacattagacagtcagtgtttggtggcaaaaatatccacgtagcaacagctcatgaagatttggcctactcttcttatgtccaccagtatagctctgggaaatttgacaatgcactatttcatgcagaaagagctattggtatcattacccacatcctacctgaagatcatcttcttttggcttcttcaaagagggtgaaagcacttattttagaggagattgcaattgattgtcataataaggaaactgaacagaggctgcttcaagaagctcatgatttgcacctgtcttcactccaactagctaaaaaagcttttggggaatttaatgtacagactgcaaaacactatggaaaccttggaagactttatcagtcaatgagaaaatttaaggaagctgaagaaatgcacatcaaagcaattcagattaaagaacaacttcttggtcaagaagattatgaagtagccctttcagtgggacatctggcttctttatataattatgacatgaatcagtatgaaaatgctgagaaactttatttgcgatctatagcaattgggaagaaactttttggtgagggctacagtggactagaatatgattatcgaggtctcattaaactttacaactccattggaaattacgagaaagtgtttgaatatcacaatgttctgtctaactggaaccggttgcgagatcggcaatattcagtgacagatgctcttgaagatgtcagcaccagcccccagtccactgaagaagtggtgcagtccttcctgatttctcagaatgtcgagggaccgagctgctga

SEQ ID NO: 3 - protein, 919 aa, Artificial Sequence
  Description of Artificial Sequence/note = synthetic construct
MEVQLGLGRVYPRPPSKTYRGAFQNLFQSVREVIQNPGPRHPEAASAAPPGASLLLLQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 4 - DNA, 4321 bp, Artificial Sequence
  Description of Artificial Sequence/note = synthetic construct
cgagatcccggggagccagcttgctgggagagcgggacggtccggagcaagcccacaggcagaggaggcgacagagggaaaaagggccgagctagccgctccagtgctgtacaggagccgaagggacgcaccacgccagccccagcccggctccagcgacagccaacgcctcttgcagcgcggcggcttcgaagccgccgcccggagctgccctttcctcttcggtgaagtttttaaaagctgctaaagactcggaggaagcaaggaaagtgcctggtaggactgacggctgcctttgtcctcctcctctccaccccgcctccccccaccctgccttccccccctcccccgtcttctctcccgcagctgcctcagtcggctactctcagccaacccccctcaccacccttctccccacccgcccccccgcccccgtcggcccagcgctgccagcccgagtttgcagagaggtaactccctttggctgcgagcgggcgagctagctgcacattgcaaagaaggctcttaggagccaggcgactggggagcggcttcagcactgcagccacgacccgcctggttagaattccggcggagagaaccctctgttttcccccactctctctccacctcctcctgccttccccaccccgagtgcggagcagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaaccctatttccccaccccagctcatgccccctttcagatgtcttctgcctgttataactctgcactactcctctgcagtgccttggggaatttcctctattgatgtacagtctgtcatgaacatgttcctgaattctatttgctgggctttttttttctctttctctcctttctttttcttcttccctccctatctaaccctcccatggcaccttcagactttgcttcccattgtggctcctatctgtgttttgaatggtgttgtatgcctttaaatctgtgatgatcctcatatggcccagtgtcaagttgtgcttgtttacagcactactctgtgccagccacacaaacgtttacttatcttatgccacgggaagtttagagagctaagattatctggggaaatcaaaacaaaaaacaagcaaacaaaaaaaaaa

SEQ ID NO: 5 - protein, 433 aa, Artificial Sequence
  Description of Artificial Sequence/note = synthetic construct
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKDSSGTGHFTSGVRVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 6 - DNA, 1639 bp, Artificial Sequence
  Description of Artificial Sequence/note = synthetic construct
atcatctatatgttaaatatccgtgccgatctgtcttgaaggagaaatatatcgcttgttttgttttttatagtatacaaaaggagtgaaaagccaagaggacgaagtctttttctttttcttctgtgggagaacttaatgctgcatttatcgttaacctaacaccccaacataaagacaaaaggaagaaaaggaggaaggaaggaaaaggtgattcgcgaagagagtgatcatgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggattcgtcaggaacaggacatttcacctcaggagtgcgggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacctgaacagtcccgagcagccagctgcacaggaaaaaccaccagttacttgagtgtcactcagcaacactggtcacgtttggaaagaatattaaaaaaaaaaaaaaaaaa

SEQ ID NO: 7 - protein, 391 aa, Artificial Sequence
  Description of Artificial Sequence/note = synthetic construct
MKCLVTGGNVKVLGKAVHSLSRIGDELYLEPLEDGLSLRTVNSSRSAYACFLFAPLFFQQYQAATPGQDLLRCKILMKSFLSVFRSLAMLEKTVEKCCISLNGRSSRLVVQLHCKFGVRKTHNLSFQDCESLQAVFDPASCPHMLRAPARVLGEAVLPFSPALAEVTLGIGRGRRVILRSYHEEEADSTAKAMVTEMCLGEEDFQQLQAQEGVAITFCLKEFRGLLSFAESANLNLSIHFDAPGRPAIFTIKDSLLDGHFVLATLSDTDSHSQDLGSPERHQPVPQLQAHSTPHPDDFANDDIDSYMIAMETTIGNEGSRVLPSISLSPGPQPPKSPGPHSEEEDEAEPSTVPGTPPPKKFRSLFFGSILAPVRSPQGPSPVLAEDSEGEG

SEQ ID NO: 8 - DNA, 2102 bp, Artificial Sequence
  Description of Artificial Sequence/note = synthetic construct
gcgcgggaagggaccccggacccggaggtcgcggagagctgggcagtgttggccgctggcggagcgctggggcagcatgaagtgcctggtcacgggcggcaacgtgaaggtgctcggcaaggccgtccactccctgtcccgcatcggggacgagctctacctggaacccttggaggacgggctctccctccggacggtgaactcctcccgctctgcctatgcctgctttctctttgccccgctcttcttccagcaataccaggcagccacccctggtcaggacctgctgcgctgtaagatcctgatgaagtctttcctgtctgtcttccgctcactggcgatgctggagaagacggtggaaaaatgctgcatctccctgaatggccggagcagccgcctggtggtccagctgcattgcaagttcggggtgcggaagactcacaacctgtccttccaggactgtgagtccctgcaggccgtcttcgacccagcctcgtgcccccacatgctccgcgccccagcacgggttctgggggaggctgttctgcccttctctcctgcactggctgaagtgacgctgggcattggccgtggccgcagggtcatcctgcgcagctaccacgaggaggaggcagacagcactgccaaagccatggtgactgagatgtgccttggagaggaggatttccagcagctgcaggcccaggaaggggtggccatcactttctgcctcaaggaattccgggggctcctgagctttgcagagtcagcaaacttgaatcttagcattcattttgatgctccaggcaggcccgccatcttcaccatcaaggactctttgctggacggccactttgtcttggccacactctcagacaccgactcgcactcccaggacctgggctccccagagcgtcaccagccagtgcctcagctccaggctcacagcacaccccacccggacgactttgccaatgacgacattgactcttacatgatcgccatggaaaccactataggcaatgagggctcgcgggtgctgccctccatttccctttcacctggcccccagccccccaagagccccggtccccactccgaggaggaagatgaggctgagcccagtacagtgcctgggactcccccacccaagaagttccgctcactgttcttcggctccatcctggcccctgtacgctccccccagggccccagccctgtgctggcggaagacagtgagggtgaaggctgaaccaagaacctgaagcctgtacccagaggccttggactagacgaagccccagccagtggcagaactgggtctctcagccctggggatcagaaaggtgggcttgctggagctgagctgtttcactgcctctcgcaggccccagctggctgtcactgtaaagctgtcccacagcggtcgggcctgggccgttatctccccacaacccccagccaatcaggactttccagacttggccctgaactactgacgttcctacctcttatttctcattgagcctcaggctatactccagctggccaaggctggaaacctgtctccctcaggctcaccttcctaaggaaaatgtcatagtaggtgctgctggcccctggtgatccagcttctctgccaatcatgacctgttccttcctgaagtcctgggcatgcatctgggacccccgtggagctgacaagttttccttgctttcctgatactctttggcgctgacttggaattctaagagccttggacccgagtgtgtggctagggttgccctggctggggcccggtgccgagactcccaagcggctctgtgcagaagagctgccaggcagtgtcttagatgtgagacggaggccatggcgagaatccagctttgacctttattcaagagaccagatgggttgccccaggatccggctgccagccctgaggccaagcacggctggagacccacgacctggcctgccgttgccctgagctgcagcctcggccccaggatcctgctcacagtcaccgcaggtgcaggcaggaagcagccctgggggactggacgctgctattgattcattaaaaaaagaaaagaaaaat

SEQ ID NO: 9 - DNA, 22 bp, Artificial Sequence
  Description of Artificial Sequence/note = synthetic construct
gggcccctggatggatagctac

SEQ ID NO: 10 - DNA, 21 bp, Artificial Sequence
  Description of Artificial Sequence/note = synthetic construct
gtagctatccatccaggggcc

SEQ ID NO: 11 - DNA, 49 bp, Artificial Sequence
  Description of Artificial Sequence/note = synthetic construct
gggcccctggatggatagctacctcgaggtagctatccatccaggggcc

SEQ ID NO: 12 - DNA, 54 bp, Artificial Sequence
  Description of Artificial Sequence/note = synthetic construct
tttttgggcccctggatggatagctacctcgaggtagctatccatccaggggcc

SEQ ID NO: 13 - protein, 483 aa, Artificial Sequence
  Description of Artificial Sequence/note = synthetic construct
MATIEEIAHQIIEQQMGEIVTEQQTGQKIQIVTALDHNTQGKQFILTNHDGSTPSKVILARQDSTPGKVFLTTPDAAGVNQLFFTTPDLSAQHLQLLTDNSPDQGPNKVFDLCVVCGDKASGRHYGAVTCEGCKGFFKRSIRKNLVYSCRGSKDCIINKHHRNRCQYCRLQRCIAFGMKQDSVQCERKPIEVSREKSSNCAASTEKIYIRKDLRSPLTATPTFVTDSESTRSTGLLDSGMFMNIHPSGVKTESAVLMTSDKAESCQGDLSTLANVVTSLANLGKTKDLSQNSNEMSMIESLSNDDTSLCEFQEMQTNGDVSRAFDTLAKALNPGESTACQSSVAGMEGSVHLITGDSSINYTEKEGPLLSDSHVAFRLTMPSPMPEYLNVHYIGESASRLLFLSMHWALSIPSFQALGQENSISLVKAYWNELFTLGLAQCWQVMNVATILATFVNCLHNSLQQDAKVIAALIHFTRRAITDL

SEQ ID NO: 14 - protein, 596 aa, Artificial Sequence
  Description of Artificial Sequence/note = synthetic construct
MTSPSPRIQIISTDSAVASPQRIQIVTDQQTGQKIQIVTAVDASGSPKQQFILTSPDGAGTGKVILASPETSSAKQLIFTTSDNLVPGRIQIVTDSASVERLLGKTDVQRPQVVEYCVVCGDKASGRHYGAVSCEGCKGFFKRSVRKNLTYSCRSNQDCIINKHHRNRCQFCRLKKCLEMGMKMESVQSERKPFDVQREKPSNCAASTEKIYIRKDLRSPLIATPTFVADKDGARQTGLLDPGMLVNIQQPLIREDGTVLLATDSKAETSQGALGTLANVVTSLANLSESLNNGDTSEIQPEDQSASEITRAFDTLAKALNTTDSSSSPSLADGIDTSGGGSIHVISRDQSTPIIEVEGPLLSDTHVTFKLTMPSPMPEYLNVHYICESASRLLFLSMHWARSIPAFQALGQDCNTSLVRACWNELFTLGLAQCAQVMSLSTILAAIVNHLQNSIQEDKLSGDRIKQVMEHIWKLQEFCNSMAKLDIDGYEYAYLKAIVLFSPDHPGLTSTSQIEKFQEKAQMELQDYVQKTYSEDTYRLARILVRLPALRLMSSNITEELFFTGLIGNVSIDSIIPYILKMETAEYNGQITGASL

SEQ ID NO: 15 - DNA, 2029 bp, Artificial Sequence
  Description of Artificial Sequence/note = synthetic construct
gaattcgggcccgtcggctttcttcaaccctctcttcccggagcgcccccaatccacgagtggcagccgcgggactgtcgcgtcggcgcccgacgcggagtcagcaggggcgaaaagcggtagatcatggcaaccatagaagaaattgcacatcaaattattgaacaacagatgggagagattgttacagagcagcaaactgggcagaaaatccagattgtgacagcacttgatcataatacccaaggcaagcagttcattctgacaaatcacgacggctctactccaagcaaagtcattctggccaggcaagattccactccgggaaaagttttccttacaactccagatgcagcaggtgtcaaccagttattttttaccactcctgatctgtctgcacaacacctgcagctcctaacagataattctccagaccaaggaccaaataaggtttttgatctttgcgtagtatgtggagacaaagcatcaggacgtcattatggagcagtaacttgtgaaggctgcaaaggattttttaaaagaagcatccgaaaaaatttagtatattcatgtcgaggatcaaaggattgtattattaataagcaccaccgaaaccgctgtcaatactgcaggttacagagatgtattgcgtttggaatgaagcaagactctgtccaatgtgaaagaaaacccattgaagtatcacgagaaaaatcttccaactgtgccgcttcaacagaaaaaatctatatccgaaaggaccttcgtagcccattaactgcaactccaacttttgtaacagatagtgaaagtacaaggtcaacaggactgttagattcaggaatgttcatgaatattcatccatctggagtaaaaactgagtcagctgtgctgatgacatcagataaggctgaatcatgtcagggagatttaagtacattggccaatgtggttacatcattagcgaatcttggaaaaactaaagatctttctcaaaatagtaatgaaatgtctatgattgaaagcttaagcaatgatgatacctctttgtgtgaatttcaagaaatgcagaccaacggtgatgtttcaagggcatttgacactcttgcaaaagcattgaatcctggagagagcacagcctgccagagctcagtagcgggcatggaaggaagtgtacacctaatcactggagattcaagcataaattacaccgaaaaagaggggccacttctcagcgattcacatgtagctttcaggctcaccatgccttctcctatgcctgagtacctgaatgtgcactacattggggagtctgcctccagactgctgttcttatcaatgcactgggcactttcgattccttctttccaggctctagggcaagaaaacagcatatcactggtgaaagcttactggaatgaactttttactcttggtcttgcccagtgctggcaagtgatgaatgtagcaactatattagcaacatttgtcaattgtcttcacaatagtcttcaacaagatgccaaggtaattgcagccctcattcatttcacaagacgagcaatcactgatttataaatgcttaactatagaatggcttatgactacccaaaacagtgccccatcaacaaatggggaaaattgccttttgagctcaggaataatttataaattggggactaccttttagttctttagcatattctatttcttattgttttatataatttttaaatcatttgcttcctccttatgtttaacagcagaggggtaatcaccttaaaatgtcatcaaaaatagatctactagaaggcagcatcacattcccatcttacttatggactcctacccctggttcatgtcttatatgcctgtaatggttataaagcctaccttcaggaaagctatggttgactaattactaatggatgggttttaaacatgtccctctacaataaattaaaatctttcaatgtttgaatataatgtggaggtgtttacctgagggcctctctatctccccgaattc

SEQ ID NO: 16 - DNA, 6450 bp, Artificial Sequence
  Description of Artificial Sequence/note = synthetic construct
gagttgtgcctggagtgatgtttaagccaatgtcagggcaaggcaacagtccctggccgtcctccagcacctttgtaatgcatatgagctcgggagaccagtacttaaagttggaggcccgggagcccaggagctggcggagggcgttcgtcctgggagctgcacttgctccgtcgggtcgccggcttcaccggaccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctgagagctccctggctcccacacggttcagataatccctgctgcattttaccctcatcatgcaccactttagccaaattctgtctcctgcatacactccggcatgcatccaacaccaatggctttctagatgagtggccattcatttgcttgctcagttcttagtggcacatcttctgtcttctgttgggaacagccaaagggattccaaggctaaatctttgtaacagctctctttcccccttgctatgttactaagcgtgaggattcccgtagctcttcacagctgaactcagtctatgggttggggctcagataactctgtgcatttaagctacttgtagagacccaggcctggagagtagacattttgcctctgataagcactttttaaatggctctaagaataagccacagcaaagaatttaaagtggctcctttaattggtgacttggagaaagctaggtcaagggtttattatagcaccctcttgtattcctatggcaatgcatccttttatgaaagtggtacaccttaaagcttttatatgactgtagcagagtatctggtgattgtcaattcacttccccctataggaatacaaggggccacacagggaaggcagatcccctagttggccaagacttattttaacttgatacactgcagattcagagtgtcctgaagctctgcctctggctttccggtcatgggttccagttaattcatgcctcccatggacctatggagagcaacaagttgatcttagttaagtctccctatatgagggataagttcctgatttttgtttttatttttgtgttacaaaagaaagccctccctccctgaacttgcagtaaggtcagcttcaggacctgttccagtgggcactgtacttggatcttcccggcgtgtgtgtgccttacacaggggtgaactgttcactgtggtgatgcatgatgagggtaaatggtagttgaaaggagcaggggccctggtgttgcatttagccctggggcatggagctgaacagtacttgtgcaggattgttgtggctactagagaacaagagggaaagtagggcagaaactggatacagttctgagcacagccagacttgctcaggtggccctgcacaggctgcagctacctaggaacattccttgcagaccccgcattgcctttgggggtgccctgggatccctggggtagtccagctcttattcatttcccagcgtggccctggttggaagaagcagctgtcaagttgtagacagctgtgttcctacaattggcccagcaccctggggcacgggagaagggtggggaccgttgctgtcactactcaggctgactggggcctggtcagattacgtatgcccttggtggtttagagataatccaaaatcagggtttggtttggggaagaaaatcctcccccttcctcccccgccccgttccctaccgcctccactcctgccagctcatttccttcaatttcctttgacctataggctaaaaaagaaaggctcattccagccacagggcagccttccctgggcctttgcttctctagcacaattatgggttacttcctttttcttaacaaaaaagaatgtttgatttcctctgggtgaccttattgtctgtaattgaaaccctattgagaggtgatgtctgtgttagccaatgacccaggtagctgctcgggcttctcttggtatgtcttgtttggaaaagtggatttcattcatttctgattgtccagttaagtgatcaccaaaggactgagaatctgggagggcaaaaaaaaaaaaaaaagtttttatgtgcacttaaatttggggacaattttatgtatctgtgttaaggatatgcttaagaacataattcttttgttgctgtttgtttaagaagcaccttagtttgtttaagaagcaccttatatagtataatatatatttttttgaaattacattgcttgtttatcagacaattgaatgtagtaattctgttctggatttaatttgactgggttaacatgcaaaaaccaaggaaaaatatttagtttttttttttttttttgtatacttttcaagctaccttgtcatgtatacagtcatttatgcctaaagcctggtgattattcatttaaatgaagatcacatttcatatcaacttttgtatccacagtagacaaaatagcactaatccagatgcctattgttggatattgaatgacagacaatcttatgtagcaaagattatgcctgaaaaggaaaattattcagggcagctaattttgcttttaccaaaatatcagtagtaatatttttggacagtagctaatgggtcagtgggttctttttaatgtttatacttagattttcttttaaaaaaattaaaataaaacaaaaaaaatttctaggactagacgatgtaataccagctaaagccaaacaattatacagtggaaggttttacattattcatccaatgtgtttctattcatgttaagatactactacatttgaagtgggcagagaacatcagatgattgaaatgttcgcccaggggtctccagcaactttggaaatctctttgtatttttacttgaagtgccactaatggacagcagatattttctggctgatgttggtattgggtgtaggaacatgatttaaaaaaaaaactcttgcctctgctttcccccactctgaggcaagttaaaatgtaaaagatgtgatttatctggggggctcaggtatggtggggaagtggattcaggaatctggggaatggcaaatatattaagaagagtattgaaagtatttggaggaaaatggttaattctgggtgtgcaccaaggttcagtagagtccacttctgccctggagaccacaaatcaactagctccatttacagccatttctaaaatggcagcttcagttctagagaagaaagaacaacatcagcagtaaagtccatggaatagctagtggtctgtgtttcttttcgccattgcctagcttgccgtaatgattctataatgccatcatgcagcaattatgagaggctaggtcatccaaagagaagaccctatcaatgtaggttgcaaaatctaacccctaaggaagtgcagtctttgatttgatttccctagtaaccttgcagatatgtttaaccaagccatagcccatgccttttgagggctgaacaaataagggacttactgataatttacttttgatcacattaaggtgttctcaccttgaaatcttatacactgaaatggccattgatttaggccactggcttagagtactccttcccctgcatgacactgattacaaatactttcctattcatactttccaattatgagatggactgtgggtactgggagtgatcactaacaccatagtaatgtctaatattcacaggcagatctgcttggggaagctagttatgtgaaaggcaaataaagtcatacagtagctcaaaaggcaaccataattctctttggtgcaagtcttgggagcgtgatctagattacactgcaccattcccaagttaatcccctgaaaacttactctcaactggagcaaatgaactttggtcccaaatatccatcttttcagtagcgttaattatgctctgtttccaactgcatttcctttccaattgaattaaagtgtggcctcgtttttagtcatttaaaattgttttctaagtaattgctgcctctattatggcacttcaattttgcactgtcttttgagattcaagaaaaatttctattcatttttttgcatccaattgtgcctgaacttttaaaatatgtaaatgctgccatgttccaaacccatcgtcagtgtgtgtgtttagagctgtgcaccctagaaacaacatacttgtcccatgagcaggtgcctgagacacagacccctttgcattcacagagaggtcattggttatagagacttgaattaataagtgacattatgccagtttctgttctctcacaggtgataaacaatgctttttgtgcactacatactcttcagtgtagagctcttgttttatgggaaaaggctcaaatgccaaattgtgtttgatggattaatatgcccttttgccgatgcatactattactgatgtgactcggttttgtcgcagctttgctttgtttaatgaaacacacttgtaaacctcttttgcactttgaaaaagaatccagcgggatgctcgagcacctgtaaacaattttctcaacctatttgatgttcaaataaagaattaaact

SEQ ID NO: 17 - DNA, 23 bp, Artificial Sequence
  Description of Artificial Sequence/note = synthetic construct
cgctgtaagatcctgatgaagtc

SEQ ID NO: 18 - DNA, 19 bp, Artificial Sequence
  Description of Artificial Sequence/note = synthetic construct
tgcctcctcctcgtggtag

SEQ ID NO: 19 - DNA, 21 bp, Artificial Sequence
  Description of Artificial Sequence/note = synthetic construct
tgccttccttggatgtggtag

SEQ ID NO: 20 - DNA, 21 bp, Artificial Sequence
  Description of Artificial Sequence/note = synthetic construct
cgtctgccctatcaactttcg

SEQ ID NO: 21 - protein, 920 aa, Artificial Sequence
  Description of Artificial Sequence/note = synthetic construct
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFHRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 22 - protein, 12 aa, Artificial Sequence
  Description of Artificial Sequence/note = synthetic construct
PKKFRSLFFGSI

SEQ ID NO: 23 - protein, 19 aa, Artificial Sequence
  Description of Artificial Sequence/note = synthetic construct
HPTHSSRLWELLMEATPTM

(19) United States
(12) Patent Application Publication
(10) Pub. No.: US 2008/0050387 A1
(43) Pub. Date: Feb. 28, 2008

(54) Non-Androgen Dependent Roles for Androgen Receptor and Non-Androgen Related Inhibitors of Androgen Receptor

(76) Inventor: Chawnshang Chang, Pittsford, NY (US)

Correspondence Address: NEEDLE & ROSENBERG, P.C., SUITE 1000, 999 PEACHTREE STREET, ATLANTA, GA, 30309-3915, US

(21) Appl. No.: 10/582,292

(22) PCT Filed: Dec. 13, 2004
(86) PCT No.: PCT/US04/41631

§ 371 Date: May 31, 2007

Related U.S. Application Data
(60) Provisional application No. 60/529,011, filed on Dec. 12, 2003.

(51) Int. Cl. A61K 31/70; A61K 38/00; A61K 39/395; A61P 43/00; C12Q 1/68; G01N 33/574

(52) U.S. Cl. 424/143.1; 424/130.1; 435/6; 435/7.23; 514/12; 514/44

(57) ABSTRACT

Disclosed are compositions and methods for modulating AR activity, such as non-androgen dependent AR activity. Also disclosed are compositions and methods for diagnosing beast cancer and for inhibiting breast cancer growth. In addition, disclosed are methods for identifying molecules that inhibit AR in non-androgen dependent ways.

### I. ACKNOWLEDGEMENTS

This invention was made with government support under federal grants DK60905 and DK60948 awarded by the NIH and Army grants DAMD17-02-1-0557 and NY-CO 17047. The Government has certain rights to this invention.

### II. BACKGROUND OF THE INVENTION

Androgen receptor (AR) is a member of the steroid hormone superfamily of nuclear receptors. Androgen receptor has been implicated in many cancers in an androgen dependent way. Disclosed herein androgen receptor is also involved in the development of breast tissue and in the progression of breast cancers in androgen independent ways. Furthermore, while antiandrogens, such as hydroxyflutamide have been used to treat AR dependent cancers for many years, disclosed are molecules that inhibit AR activity, particularly androgen independent AR activity. The disclosed molecules and their interactions between androgen receptor, as well as the information that androgen receptor can have effects in proliferation of cancers through a non-androgen mechanism provide for methods of identifying compositions which modulate or mimic this activity, as well as methods of modulating AR activity itself.

### III. SUMMARY OF THE INVENTION

In accordance with the purposes of this invention, as embodied and broadly described herein, this invention, in one aspect, relates to compositions and methods related to androgen receptor and methods of inhibiting cancer.

It is to be understood that both the foregoing general description and the following detailed description are exemplary and explanatory only and are not restrictive of the invention, as claimed.

### IV. BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 shows the generation and characterization of immature female AR^{-/-} mice.

FIG. 1A shows gene targeting strategies. To generate female AR^{-/-} mice, a Cre-lox strategy for conditional knockout is applied. The Cre-lox system utilizes the expression of P1 phage Cre recombinase (Cre) to catalyze the excision of DNA located between flanking lox sites. FIG. 1B shows the breeding strategy of female AR^{-/-} mice and genotyping of female AR−/− mice. Using the Cre-lox strategy, the targeted exon 2 of AR is not disrupted but floxed in the male mice. Thus the AR functions normally in male mice, which can be bred with female AR^{+/-} ACTB Cre⁺ mice and generate homozygous female AR^{-/-} mice. For examining the X chromosome with floxed AR, primer “select” and primer “2-3” are used. “Select” is located in the intron 1 with sequence: 5′-GTTGATACCTTAACCTCTGC-3′. “2-3” is the 3′ end primer which is located in the exon 2 with the sequence 5′-TTCAGCGGCTCTTTTGAAG-3′. This pair of primers will amplify a product with 444 bp for floxed AR, 410 bp for wtAR. For examining the AR knockout (ARKO) locus, primer “select” and “2-9” were used. “2-9” is located in intron 2 with sequence: 5′-CCTACATGTACTGTGAGAGG-3′. The PCR product size from this pair of primers would be 238 bp for ARKO allele and 580 bp for wt AR allele. The expression of Cre and internal control IL-2 were also confirmed by PCR genotyping. FIG. 1C shows the defects in the ductal development of mammary gland in immature female AR^{-/-} mice. Whole breast mounts from 4-wk-old female AR^{-/-} mice show lessened extension of mammary ducts, as compared with age-matched AR^{+/+} mice. FIG. 1D shows the decrease in the percentage of BrdU-positive staining (brown color) are observed in both 4- and 6-wk-old mice. FIG. 1E shows statistic results of the distance of ductal extension indicating the retarded growth of mammary glands in female AR^{-/-} mice (left panel). Statistic results of BrdU signal (right panel) (n−5 for each group). FIG. 1F shows the number of Cap cells (indicated as arrows) in TEB of AR^{-/-} mice is less than that in the AR mice.

FIG. 2 shows that AR^{-/-} mammary glands show the defects of the terminal branching and alveologenesis during maturity and pregnancy. Whole breast mounts from 8-, 16-, 20-wk-old mature and 8-wk-old pregnant AR^{+/+} and AR^{-/-} female were examined. In (A-C) Note less secondary and tertiary terminal branching in AR^{-/-} mice, compared with AR^{+/+} mice. Also, the ductal spaces are reduced in AR^{-/-} mammary glands. FIG. 2C shows early degeneration occurs in AR^{+/+} mammary glands at 20-wk-old mice. FIG. 2D shows the decreased milk producing lobuloalveolar development in the 8-wk-old pregnant AR^{-/-} mice. FIG. 2E shows that using H & E staining, the results indicate that the shrunken ductal space occurs in some AR^{-/-} mammary glands in 16- to 20-wk-old mice (n=4 for each group).

FIG. 3 shows the reduced MAPK activity and mRNA expression of IGF-IR, HGF, and Efp in AR^{-/-}— mammary glands. FIGS. 3A and 3B shows that reduced MAPK activities (α-phospho-MAPK (p), brown color) were observed in AR^{-/-} mammary glands of 6-wk-old and 4-wk-old mice (results from 6-wk-old mice were shown as representative). FIG. 3A represents ductal structure, the positive MAPK stainings are mainly located on luminal epithelial cells; (B) represents lobule part. FIG. 3C shows the mRNA expression of IGF-IR, but not IGF-I, is reduced in AR^{-/-} mice. Total RNA was extracted from 4-wk-old AR+/+ and AR−/− mice and quantitated by real-time RT-PCR. Cyclin D1, a proliferation indicator, is also reduced in mammary gland of female AR^{-/-} mice. FIG. 3D shows the mRNA expressions of two ER target genes, HGF and Efp, are reduced in AR^{-/-} mice. Total RNA was extracted from 5-wk-old A^{+/+} and AR^{-/-} mice injected with E2 (n=5 for each group).

FIG. 4 shows targeted deletion of AR gene in MCF7 cells results in severe defects in cell proliferation and colony formation. FIG. 4A shows the schematic diagram of the strategy of targeting AR genes in MCF7 cells. FIG. 4B shows genotyping by Southern blot analysis. Genomic DNA extracted from neomycin-resistant clones was digested with XbaI. The untargeted and targeted loci produced approximately 9.0-kb and 3.5-kb bands, respectively. FIG. 4C shows the AR protein is ablated in AR^{-/-} MCF7 cells. FIG. 4D shows the ligand-activated transcriptional activity of AR is reduced in AR^{+/+} MCF7 cells and abrogated in AR^{-/-} MCF7 cells, compared with AR^{+/+} MCF7 cells. FIG. 4E shows the proliferation of AR^{-/-} MCF7 cells is reduced in medium containing 10% normal serum (left panel) or 10% CDS serum with ethanol (e) or 10⁻¹⁰ E2 (right panel), compared with AR^{+/+} MCF7 cells, using MTT proliferation assay. FIG. 4F shows the soft-agar colony formation capacity of AR^{-/-} MCF7 cells is reduced, compared with AR^{+/+} MCF7 cells.

FIG. 5 shows AR is essential for growth factor and estrogen signaling pathway. FIG. 5A shows that growth factor-induced cell proliferation is impaired in AR^{-/-} MCF7 cells, compared with AR^{+/+} MCF7 cells. Cultures were incubated with 0.2% serum-containing RPMI media treated with or without growth factors for 8 days. FIG. 5B shows the steady-state level of the active form of MAPK is lower in AR^{-/-} MCF7 cells than that in AR^{+/+} MCF7 cells, when cells were cultured in 1% HI-FBS-containing medium for 5 days (left panel). Growth factor-induced transcriptional activity of GAL4-Elk1 is diminished in AR^{-/-} MCF7 cells (right upper panel) and in AR^{+/+} MCF7 cells transfected with AR siRNA (right bottom panel). FIG. 5C shows the reduced MAPK activity can be restored by np-AR which expresses AR driven by natural AR promoter, np-AR can synergistically enhance EGF-induced GAL4-Elk1 transactivation. FIG. 5D shows the AR-FL-activated GAL4-Elk1 transactivation can be inhibited by a MAPK phosphatase (CL-100), a specific inhibitor U0126, dominant-negative Ras (Ras-DN) or Raf (Raf-DN). AR-FL, full-length wt AR. FIG. 5E shows the transcriptional activity of ER is reduced in AR^{-/-} MCF7 cells, compared with AR^{+/+} MCF7 cells. FIG. 5F shows that the reduced ER activity in AR^{-/-} MCF7 cells can be restored by np-AR. pG5-luc and ERE-Luc were the reporters for GAL4-Elk1 and ER, respectively. 5 ng pRL-TK per well was used for internal control. Transfections were performed using SuperFect (Qiagen) according to manufacturer. Values represented are mean ±S.D. from at least four independent experiments.

FIG. 6 shows the N-terminus/DBD of AR are required for normal MAPK activation, and an AR mutant (R608K) is associated with the excessive activation of MAPK. FIG. 6A shows that reintroducing AR can enhance the reduced activation of MAPK in AR^{-/-} MCF7 cells. The N-terminus together with the DBD, but not N, DBD, LBD, or LBD-dH12 alone, are required for activating MAPK, using a transient transfection assay (middle panel) and a Western blot (bottom panel) with anti-phospho-MAPK and anti-MAPK antibodies. All of the sequences were FLAG-tagged and constructed into pCDNA3 vector (top panel, Invitrogen). V, vector alone; dH4-12, AR with deletion from helix 4 to helix 12. FIG. 6B shows that AR-R608SK-induced GAL4-Elk1 transactivation is higher than AR-FL. AR-R614H-dprm, containing a point mutation (R614H) and a deletion of proline-rich motif (dprm), has lost the ability to activate MAPK, while AR-R614H or AR-dprm still partially retains MAPK activation capacity. pG5-luc was the reporter for GAL4-Elk1. 5 ng pRL-TK per well was used for internal control. Transfections were performed using SuperFect (Qiagen) according to manufacturer. Values represented are mean ±S.D. from at least four independent experiments. FIG. 6C shows the proposed molecular mechanisms. The AR abrogation in mammary glands or mammary cancer cells retards the growth or development via the impairments of the growth factor and ER signaling pathways. The reduced ER activity, as demonstrated by the decreased target gene expression (Efp and HGF), may partly result from the impairment of the growth factors/MAPK signaling pathway. The reduced ER activity may be due to the reduction of ER activity and/or the serum level of progesterone (P) after puberty but not before puberty (asterisk). The decreased cyclin D1 expression may be caused by the impairments of both the growth factor/MAPK and ER signaling pathways. Taken together, these impaired signals may contribute to the developmental defects in mouse mammary glands of AR^{-/-} mice and in breast cancer AR^{-/-} MCF7 cells.

FIG. 7 shows the identification of ARA67 as ARN interacting protein using CytoTrap Sos system. FIG. 7(A) shows a model of CytoTrap Sos system screening strategy: target protein (ARA67) is anchored to cell membrane; hSos fused with bait protein (ARN) is recruited to the membrane through target-bait interaction, activating the Ras-signaling pathway by promoting GDP/GTP exchange; Ras activates the signaling cascade that permits mutant yeast cdc25H to grow at the restrictive temperature of 37° C. FIG. 7(B) shows the interaction of ARN and ARA67 in yeast. cdc25H yeast cells were co-transformed with different combinations of expression constructs and plated on different SD/Glu (−LU) plates. After the colonies appeared on plates incubated at a permissive temperature of 25° C., 12 colonies of each transformants were picked and spotted on SD/Gal (−LU) and SD/Glu (−LU) plates for interaction tests. In section 1, yeast cells were co-transformed with pSos vector and pMyr-ARA67 (control to eliminate the false positive clones); section 2, co-transformed with pSos-ARN and pMyr-ARA67; section 3, co-transformed with pSos-MAFB and pMyr-MAFB (positive control).

FIG. 8 shows the distribution of ARA67 mRNA in human tissues and multiple cell lines. FIG. 8(A) shows the Human MTN Blot (Clontech) was hybridized with a ³²P-labeled cDNA probe covering amino acid residues 8-140 of ARA67, and subsequently probed with β-actin. Three transcripts were detected, corresponding to the sizes of 2.5 kb, 4.41 kb and 7.5 kb. FIG. 8 (1) shows the total RNA from 13 cell lines (as indicated) were used to prepare the membrane. 18S RNA was used as RNA loading control. The membrane was hybridized with ³²P labeled probe as above.

FIG. 9 shows ARA67 and AR interact in vitro and in vivo. FIG. 9(A) shows mammalian two-hybrid assay. 0.5 μg of each pM, pVP16, pVP16-ARN, and pVP16-ARA67 were co-transformed into H1299 cells in combinations as shown. Luciferase activity of the reporter, 0.5 μg pG5-Luc, was normalized by the luciferase activity of 5 ng internal control, pRL-TK, expressed as fold increase over control. The relative reporter gene activity was compared by setting the luciferase activity of vector alone group as 1. FIG. 9(B) shows purified GST control protein and GST-ARA67 fusion protein were incubated with 5 μl [³⁵S]methionine-labeled AR, ARN, ARDBD and ARLBD in the presence and absence of 10 μM DHT. Pulled-down proteins were separated on SDS-PAGE and visualized by autoradiography. FIG. 9(C) shows COS-1 cells were co-transfected with 1.5 μg pCMV-AR and 9.0 μg pKH3-ARA67 or pKH3 vector. After transfection cells were treated with or without 10 nM DHT for 24 h before harvesting. 500 μg total cell lysate proteins from each samples were immunoprecipitated with anti-AR antibody for Western blot analysis with anti-AR and anti-HA antibody (Roche).

FIG. 10 shows ARA67 suppresses AR transactivation. FIG. 10(A) shows 100 ng pSG5-AR in combination with different doses of pSG5-ARA67 (as shown in figure) and/or pSG5 vector were transfected into H1299 cells together with 500 ng of MMTV-Luc or ARE4-Luc as reporter and 2 ng of pRL-SV40 as internal control. After transfection, cells were treated with or without 10 nM DHT for 20-24 h. FIG. 10(B) shows pSG5-AR, pSG5-ARA70 and pSG5-ARA67 were co-transfected in different combinations as shown into H1299 cells. After transfection, cells were treated with or without 10 nM DHT for 24 h, and then assayed for luciferase activity. 500 ng PSA-Luc or ARE4-Luc was used as reporter, internal control was the same as above. FIG. 10(C) shows LNCap cells were transfected with or without 6.0 μg pSG5-ARA67 (as shown) in a 100 mm cell culture dish using SuperFect tranfection kit (Clontech). 50 μg total cell lysate proteins from each sample were loaded to gel and Western blotted for AR, PSA and β-actin. FIG. 10(D) shows 100 ng pSG5-AR, pSG5-GR, and pSG5-ER were co-transfected with 500 ng pSG5-ARA67 or pSG5 vector, respectively (as shown). 500 ng MMTV-Luc was used as reporter for both AR and GR, and 500 ng ERE-Luc was used as reporter for ER. Each receptor group was treated with or without their cognate ligands as shown for 24 h, and then assayed for luciferase activity. 2 ng of pRL-SV40 was used as internal control.

FIG. 11 shows the interaction domains between ARA67 and AR and their influence on AR transactivation. FIG. 11(A) shows GST only and GST-fused ARN fragments (as shown) were incubated with [³⁵S]methionine-labeled ARA67. [³⁵S]methionine-labeled ARN (B) and AR LBD (C) were incubated with GST only and GST-fased ARA67 fragments (as shown). GST pull-down assays were performed as described. FIG. 11(D) shows H1299 cells were transfected with 100 ng of pS G5-AR in combination of 600 ng of other plasmid constructs (as shown). 300 ng of MMTV-luc was used as reporter and 3 ng of pRL-TK as internal control.

FIG. 12 shows ARA67 influences AR N/C interaction and AR protein level. FIG. 12(A) shows COS-1 cells were transfected with different plasmid constructs in a combination shown in the figure and treated with or without 10 nM DHT. DNA of pSG5-ARA67 and pSG5-SRC-1 were co-transfected with AR N/C interaction pair (VP16-ARN and Gal4-ARDL) in a ratio of 4 to 3. The assays were carried out as described in mammalian two-hybrid assay. FIG. 12(B) shows H1299 cells were co-transfected with pSG5-AR and pKH3-ARA67 or pKH3 vector in a ratio of 1 to 6. Cells were treated with or without 10 nM DHT for 24 h before harvesting. 40 μg of proteins from total cell lysate from each sample were loaded to the gel and Western blotted for AR, ARA67 (anti-HA), and β-actin.

FIG. 13 shows Histone deacetylase (HDAc) activity is not involved in ARA67 mediated suppression effect on AR. COS-1 cells were transfected with pSG5-AR in combination of 6 fold of pSG5-ARA67 or pSG5 vector. Cells were then treated with DHT and TSA as indicated in the figure. Luciferase activities of reporter MMT-Luc were assayed as described above.

FIG. 14 shows ARA67 influences the subcellular distribution of AR. FIG. 14(A) shows COS-1 cells were transfected with pCMV-AR in combination of 6 fold of pcDNA4-ARA67 or pcDNA4 vector. Immunofluorescence staining was performed as described. Arrowheads point out the cells where the AR was prevented from entering the nuclei. FIG. 14(B) shows COS-1 cells were transfected with pSG5-AR in combination of 6 fold pSG5-ARA67 or pSG5 vector, and then treated with or without 10 nM for 16-20 h. Subcellular fractionation of cells were performed as described followed by Western blotting for AR and β-actin.

FIG. 15 shows the expression and activity of GSK3β. Several cell lines were incubated with 5% FBS for 24 h. Total amount of GSK3β in 50 gg cell lysate was subjected to immunoblot analysis using anti-GSK3β antibody (top panel). Inactive form of GSK3β was detected by specific anti-phospho-GSK3β antibody (bottom panel). GSK3β is constitutively active in PC-3 and DU145 cells while its activity is inhibited in LNCaP and COS-1 cells.

FIG. 16 shows the effect of GSK3β on androgen receptor transcriptional activity. FIG. 16(A) shows the expression of GSK3β, but not the kinase-mutant GSK3β, suppressed AR transactivation in COS-1 cells. AR-negative COS-1 cells were transiently transfected using SuperFect transfection reagent (QIAGEN) with 3 gg p (ARE) 4-1 uc reporter plasmid, 100 ng pRLtk-luc as an internal control, 1 pg of AR pSGS-AR expression plasmid, and 6 pg wild type, S9A, or kinase-mutant GSK3β expression plasmids as indicated. The total amount of plasmids was adjusted to 10 μg with vector plasmids. Transfected cells were induced with 10 nM DHT for 18 h before the luciferase activities were measured. Luciferase activity was analyzed following manufacturer's instructions (Promega). The results are shown as mean ±S.D. of three independent experiments. FIG. 16(B) shows the overexpression of GSK3β inhibits AR transcriptional activity in a dose-dependent manner. COS-1 cells were transfected with increasing amounts of wild type GSK3β expression plasmids as indicated. Experiments were performed and analyzed as described in A using MMTV-Luc instead of (ARE)4-Luc reporter. The results are shown as mean ±S.D. of three independent experiments. C. Overexpression of GSK3β has no effect on human GR transcriptional activity. Experiments were performed and analyzed as described in B. D. LiCl, a specific inhibitor of GSK3β, enhances AR transactivation in PC-3 cells in the absence of DHT. Experiments were performed and analyzed as described in A.

FIG. 17 shows the suppression of AR transactivation and PSA expression by GSK-3β in LNCaP cells. FIG. 17(A) shows the LNCaP cells were transfected with wild type GSK3β for 3 h, followed by DHT treatment for 18 h. Transactivation was measured by Luciferase activity using MMTV-Luc as a reporter. The data are means ±S.D. from three independent experiments. FIG. 17(B) shows overexpression of GSK3β represses PSA promoter activity. Experiments were performed and analyzed as described in A using PSA-luc instead of MMTV-luc reporter. FIG. 17(C) shows inhibition of AR target gene PSA expression by GSK3β. LNCaP cells were transfected with wild type GSK-3 or vector. The cells were treated with ethanol or 10 nM DHT for 18 h. Total RNA was isolated and PSA mRNA level was monitored by Northern blot assay.

FIG. 18 shows the phosphorylation of AR-N and suppression of AF-1 by GSK3β. FIG. 18(A) shows that for the in vitro kinase assays, the kinase buffer contains 25 mM HEPES/pH 7.4, 10 mM MgCl2, and 1 mM dithiothreitol. Purified GSK3β was obtained from Upstate Biotechnology. The kinase reactions were performed for 30 min at 30° C. in the presence of 10 μCi [−³²P]ATP, and 10 μM ATP. The reactions were terminated by addition of 4×SDS sample buffer. The samples were boiled and loaded on 20% SDS-polyacrylamide gel electrophoresis gels followed by autoradiogram. FIG. 18(B) shows COS-1 cells were transfected with the indicated plasmids and pG5-Luc reporter. Transfected cells were cultured for 24 h before the luciferase activities were measured. The data are means ±S.D. from three independent experiments.

FIG. 19 shows GSK3β interacts with AR in vitro and in vivo. FIG. 19(A) shows GST and GST fused GSK3β were expressed in E. coli. and purified by Glutathione-Sepharose 4B beads as instructed by manufacturer (Amersham Pharmacia). 5 μl of in vitro-translated β5S]-labeled AR was incubated with the GST or GST-GSK3β bound to glutathione-Sepharose beads in a pull-down assay. After extensive washing, bead-bound protein complexes were loaded onto 8% SDS-PAGE and analyzed by Phosphorlmager. The input represents 20% amount of [³⁵S]-labeled proteins used in each pull-down assay. FIG. 19(B) shows COS-1 cells plated on 100-mm dishes were transfected with pSG5-AR and pCMV-GSK3β-HA for 24 hours. COS-1 cells were solubilized in RIPA buffer containing 0.5% NP-40 and protease inhibitors. Immunoprecipitation was performed using mouse HA antibody (1:1000) or normal mouse IgG (N-IgG) and then analyzed by Western blot with anti-AR NH27 (1:1000) or anti-GSK3β (1:1000) antibodies, followed by incubation with AP conjugate goat anti-rabbit or rabbit anti-mouse IgM antibodies, and visualized with AP conjugate kit (Bio-Rad). FIG. 19(C) shows that 500 μg of total proteins from LNCaP cells were immunoprecipitated with normal rabbit IgG or rabbit anti-AR NH27, and the immunoprecipitates were subjected to a Western blot analysis using the antibody for GSK3β and the NH27 for AR.

FIG. 20 shows that stably transfected GSK3β inhibits prostate cancer CWR22R cell growth. FIG. 20(A) shows myc-tagged S9A-GSK3β or the inducible vector, pBig, was stably transfected into prostate cancer CWR22R cells. CWR22R-S9A-GSK3β and CWR22R-pBig cells were cultured in 5% FBS for 24 hr and followed by doxycycline treatment for 16 hr. Whole cell lysates were subjected to immunoblot analysis using anti-GSK3β antibody. FIG. 20(B) shows CWR22R-pBig or CWR22R-S9A-GSK3β cells were transfected with MMTV-Luc and pRLtk-Luc for 3 hr, followed by DHT treatment for 18 hr. Transactivation was measured by Luciferase activity as described in Material and Methods. FIG. 20(C) shows growth assays were performed by the MTT method as instructed by the manufacturer (Sigma). 5×10³ CWR22-S9A-GSK3β and CWR22-pBig cells were seeded in 24-well plates and incubated in RPMI with 5% charcoal-dextran-treated fetal bovine serum for 48 h. Cells were then treated with ethanol, 10 nM DHT, and/or 2 μg/ml doxycycline as indicated. After 5 days of treatments, cells were harvested for an MTT assay. Values are the means ±S.D. of ARA70 from three independent wells of cells.

FIG. 21 shows the effect of GSK3β on the interaction between AR and ARA70. FIG. 21(A) shows GSK3β does not change AR protein amount. COS-1 cells were transfected with wild type GSK3β or mock vector as indicated. After 24 h transfection, 50 μg whole-cell extract was immunoblotted with AR antibody (NH27). FIG. 21(B) shows modulation of interaction between AR and ARA70 by GSK3β. The COS-1 cells were transfected with GAL4-ARA70 and VP 16-AR. The interaction between AR and ARA70 was determined by Luciferase assay by using pG5-Luc as a reporter.

FIG. 22 shows a simplified model for the roles of GSK3β in AR-mediated target genes transactivation.

FIG. 23 shows the isolation of bRad9 as an AR coregulator by yeast two-hybrid assay. FIG. 23(A) shows GAL4-DBD-AR-DBD-LBD fusion was used as bait. FIG. 23(B) shows the structures of the human Rad9 and hRad9 fusion protein isolated from yeast screening. FIG. 23(C) shows AH109 yeast cells were transformed with GAL4-DBD-AR-DBD-LBD and GAL4-AD fused with hRad9 (aa 327-391). Liquid β-gal assay was performed as described previously.

FIG. 24 shows hRad9 expression in human prostate. FIG. 24(A) shows a human multiple tissue Northern blot (Clontech) containing 2 μg poly (A+) mRNA from the indicated tissues was hybridized with [³²P]-labeled probes corresponding to hRad9 (top panel) and β-actin (bottom panel). FIG. 24(B) shows the expression of hRad9 proteins in prostate cancer cells. Equal amounts of (30 μg) of proteins from the indicated cell lines were analyzed by immunoblotting with anti-bRad9. FIG. 24(C) shows total RNA was isolated from clinical prostatic carcinoma. Sections of tumors and normal tissues were confirmed by hematoxylin and eosin staining. After cDNA synthesis, real time quantitative PCR was performed to analyze the hRad9 amount in tumor or normal tissues.

FIG. 25 shows that hRad9 interacts with AR in mammalian cells. FIG. 25(A) shows the interaction between AR and the hRad9 C-terminus using mammalian two-hybrid assays. PC-3 cells were transiently transfected with 0.4 μg reporter plasmid pG4-LUC, and 0.3 μg GAL4-DBD fused hRad9 constructs as indicated (upper), with or without 0.3 μg of VP16 fused AR (VP 16-AR) as indicated. After 24 h 10 nM DHT treatment, the cells were harvested for LUC assay. FIG. 25(B) shows the interaction between full length of hRad9 and AR is reduced by HF. PC-3 cells were transfected with a DNA mixture containing pG4-LUC, VP16-AR, and pCMX-GAL4-hRad9, as described in FIG. 25(A). PC-3 cells were incubated with 10⁻⁵ M HF 1 h prior to 10⁻⁸ M DHT treatment. Luciferase activities were measured after another 24 h of incubation. FIG. 25(C) shows 293T cells that overexpressed AR and Flag-hRad9 were treated with or without DHT. Cell extracts were immunoprecipitated with anti-Flag antibody followed by immunoblotting with antibody to AR. FIG. 25(D) shows CWR22R cells were prepared and immunoprecipitations were performed with the use of antibody to AR, followed by immunoblotting with antibody to hRad9.

FIG. 26 shows the mapping of the domains of AR that are responsible for bRad9 interaction. FIG. 26(A) shows AH109 yeast cells were transformed with GAL4-DBD fused with various AR domains and GAL4-AD fused with hRad9 (aa 327-391). Liquid β-gal assays were performed as described in FIG. 23(A). FIG. 26(B) shows A series of ³⁵S-labeled mtARs (upper) were incubated with purified GST-hRad9 or GST alone in the presence (closed bars) or absence (open bars) of 1 DHT. The results (lower) indicated AR LBD mediates the interaction with hRad9.

FIG. 27 shows that the FXXLF motif in hRad9 mediates the AR-hRad9 interaction. FIG. 27(A) shows mutants of hRad9 were constructed using the QuikChange kit. Mammalian two-hybrid assays were performed with PC-3 cells using 0.3 μg GAL4-f-hRad9 coding for the GAL4 DNA binding domain fused to the fragment of hRad9 isolated from yeast containing residues 327-391 with wild-type (WT) or the indicated mutant sequences. GAL4-f-hRad9 was cotransfected with the 0.4 μg pG4LUC reporter vector and 0.3 μg VP16-AR containing the residues 37 to 919. Assays were performed with PC-3 cells in the presence or absence of 10 nM DHT. FIG. 27(B) shows full length of hRad9, WT or indicated mutants were fused with GAL4-DBD and used in mammalian two-hybrid assays as described in FIG. 27A. FIG. 27(C) shows mammalian two-hybrid assays were performed with PC-3 cells by coexpressing GAL4-hRad9 peptides containing the GAL4 DNA binding domain (GAL4-DBD) and the indicated hRad9 FXXLF motif.

FIG. 28 shows that hRad9 suppresses AR transcriptional activity. FIG. 28(A) shows PC-3 cells were co-transfected with 106 ng pCMV-AR, pCDNA3-Flag vectors expressing wild type of hRad9 (WT-hRad9) or mutant of hRad9 (FXXAA-hRad9) as indicated, and MMTV-Luc reporter vector using SuperFect. phRL-tk-Luc expression vector was used as a control for transfection efficiency. Cells were treated with EtOH or DHT and then lysed for Luc activities. The MMTV-Luc reporter activity from was normalized by control Luc activity. The Luc activity relative to lane 1 was calculated, and results shown here are the mean of ±S.D, of three independent experiments. FIG. 28(B) shows CWR22R cells were transfected with indicated RNAi plasmids targeting hRad9 by electroporation. Two days after transfection, cell lyses were collected and tested by immunoblotting with antibodies to hRad9 or β-actin. CWR22R cells were transfected as FIG. 28(A) to determine the effect of blocking endogenous hRad9 on AR transcriptional activity. FIG. 28(C) shows LNCaP cell were transfected with pCDNA vector or pCDNA-hRad9 by electroporation. After 24 hr, cells were treated with EtOH or 10 nM DHT for another 48 hr and 50 μg cell extracts from LNCaP were loaded on 10% SDS-polyacrylamide gel and analyzed by Western blotting.

FIG. 29 shows hRad9 has little effect on ER- or VDR-mediated transactivation. FIG. 29(A) shows PC-3 cells were transfected with DNA mixtures of pG4-Luc, pM-f-hRad9, VP16-ERα, or VP 16-VDR as indicated GAL4-D30 and GAL4-RXRα were used as positive controls for VP16-ERα and VP16-VDR respectively. FIG. 29(B) shows PC-3 cells were transfected as in FIG. 28(A). pSG5-ERα or pSG5-VDR, and their respective reporter plasmids were used as indicated.

FIG. 30 shows the C-terminus of hRad9 interrupts AR N/C interaction. FIG. 30(A) shows the FXXLF containing fragment of hRad9 efficiently blocked the interaction between the N-terminus of AR and the AR-LBD. The upper panel shows the reconstituted AR transcription assay to determine the AR N/C interaction. PC-3 cells were transfected with MMTV-LUC, pRL-tk-Luc, AR mutants, and hRad9 as indicated. After transfection, cells were treated with 10 nM DHT for 24 hr before harvesting. The Luc activity relative to lane 1 was calculated, and results are the mean ±S.D. of three independent experiments. FIG. 30(B) shows the C-terminus, not the N-terminus, of hRad9 inhibits AR transactivation. PC-3 cells were transfected as described in FIG. 30(A), except using pCMV-AR that expresses intact AR.

FIG. 31 shows a model for the role of hRad9 in AR signaling. See text for discussion.

### V. DETAILED DESCRIPTION

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

Using three different approaches (Cre-lox conditional knockout, siRNA, and homologous recombination) to abrogate the AR function in female mice and MCF7 breast cancer cells, it was demonstrated that AR can go through interruption of MAPK activity and ER signaling to exert its essential roles for the normal mammary gland development and breast cancer growth. These results are in agreement with early reports showing that both MAPK and ER are essential factors for mammary gland development and breast cancer growth. For example, ER^{-/-} mice exhibit undeveloped mammary glands similar to those of newborn mice, indicating the essential roles of ER in the ductal growth (Couse and Korach 1999), and the loss of ER can significantly delay the onset of tumor induction in MMTV-Neu (Neu/ER^{-/-}) or -Wnt-1 (Wnt-1/ER^{-/-}) transgenic mice lacking functional ER (Bocchinfuso et al. 1999; Hewitt et al. 2002). MAPK inhibitor, PD098059, could inhibit both the mammary gland alveolar morphogenesis (Niemann et al. 1998) and Her2/Neu-, H-Ras- or C-myc-initiated mammary tumor growth (Amundadottir and Leder 1998).

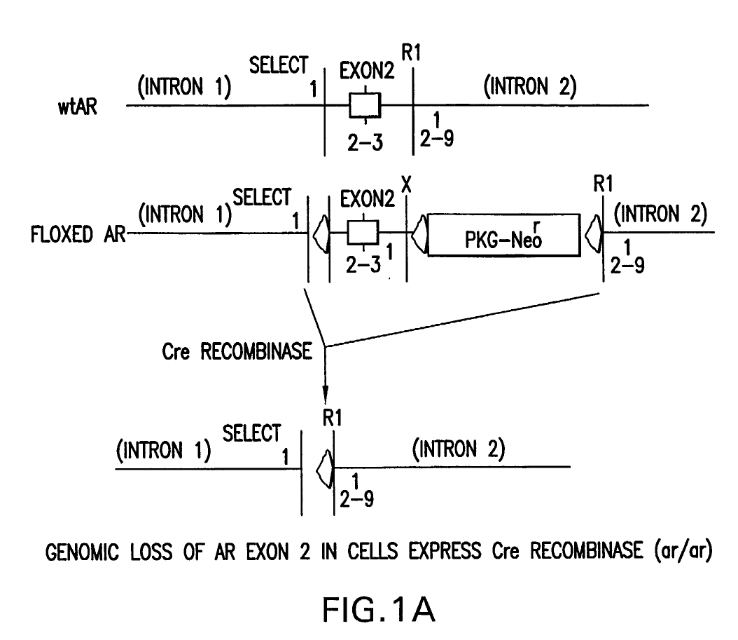

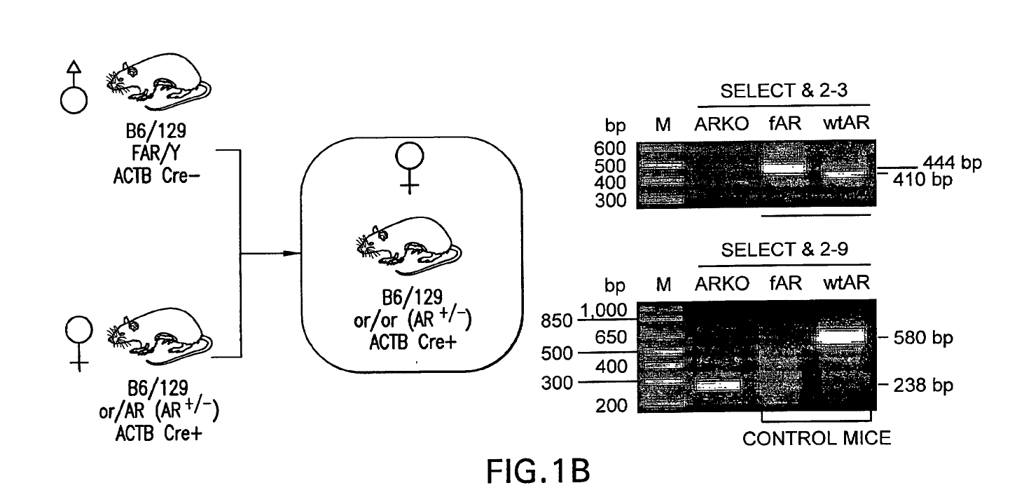

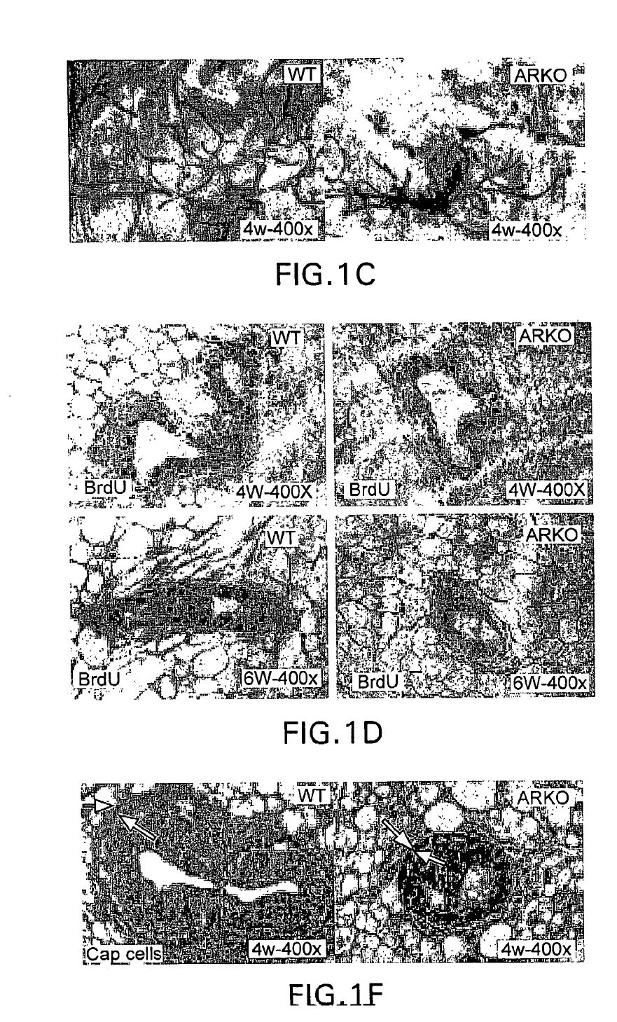

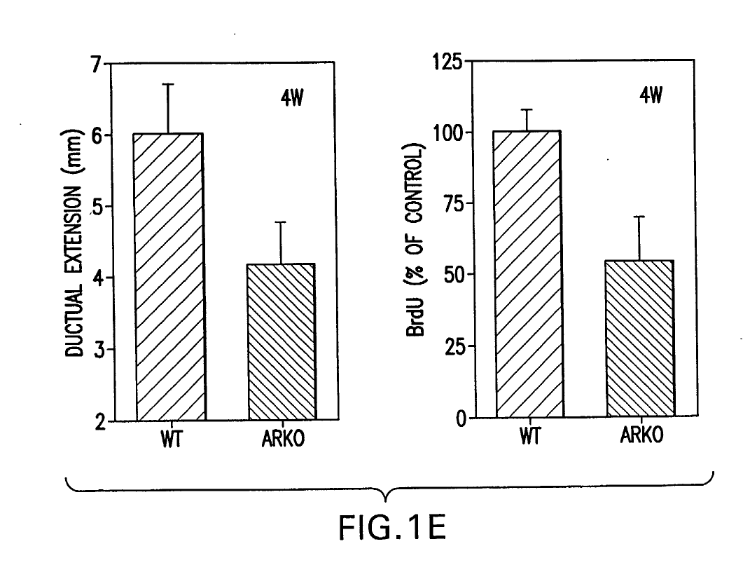

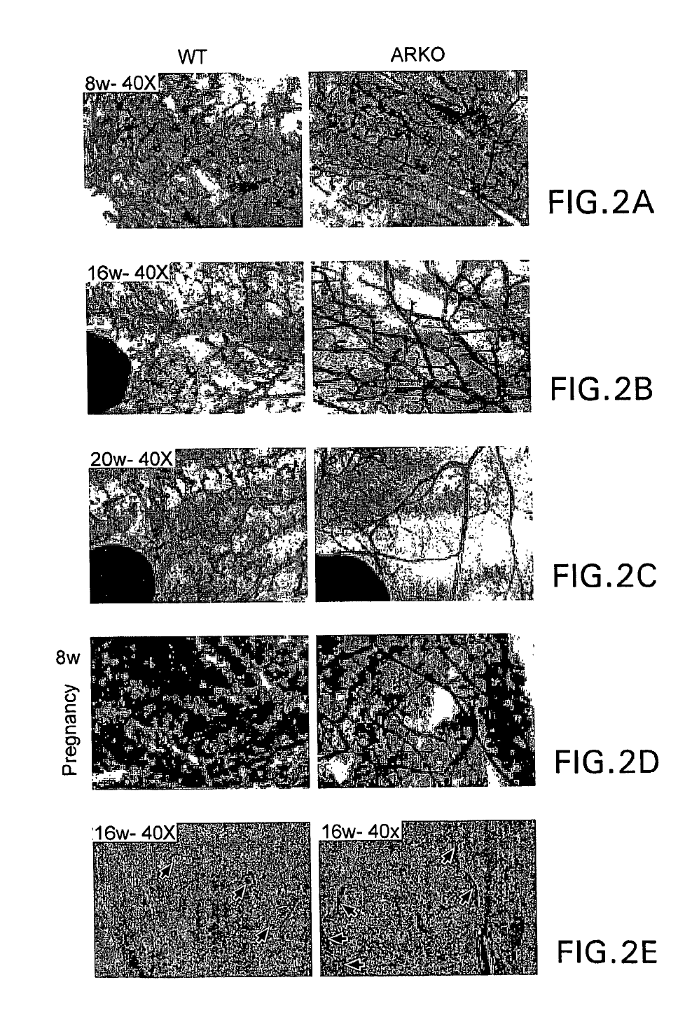

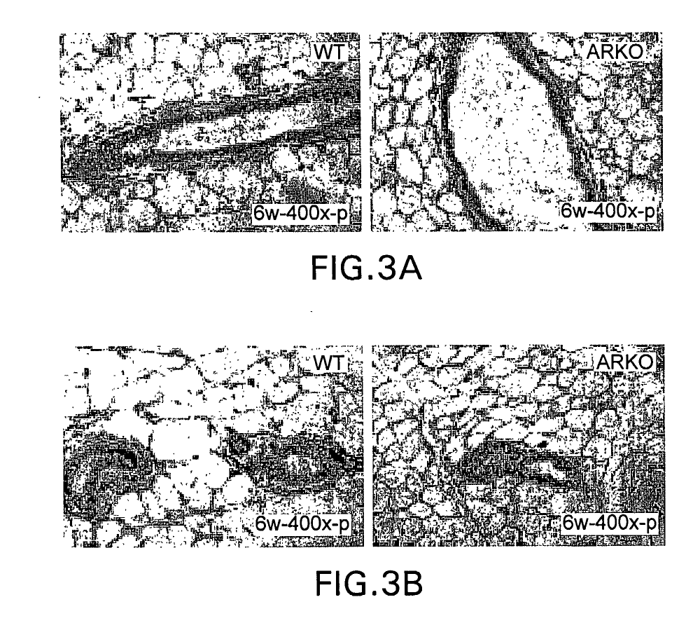

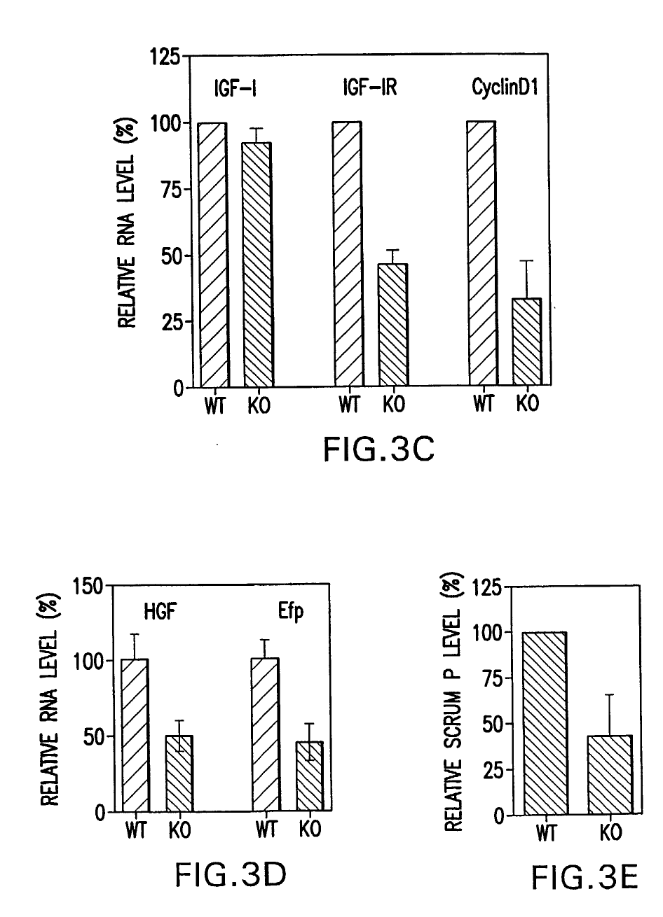

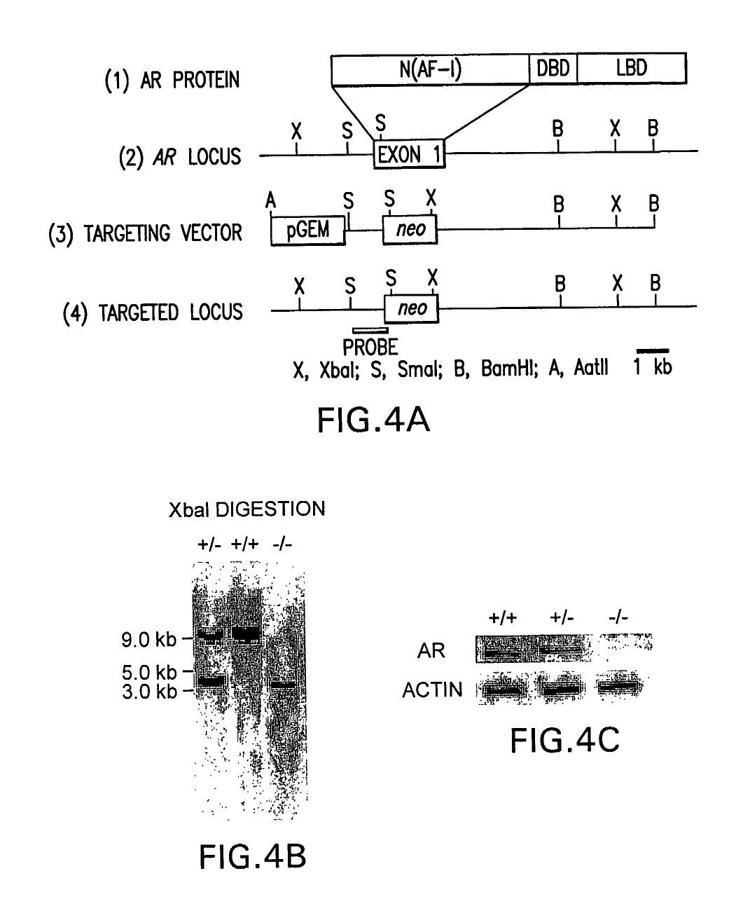

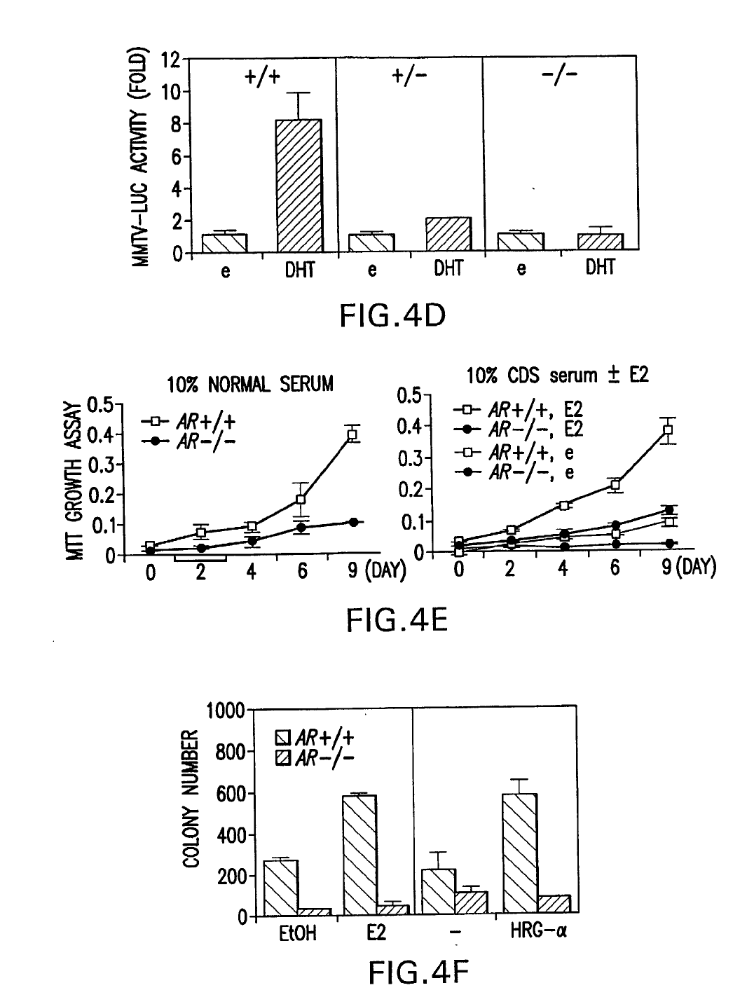

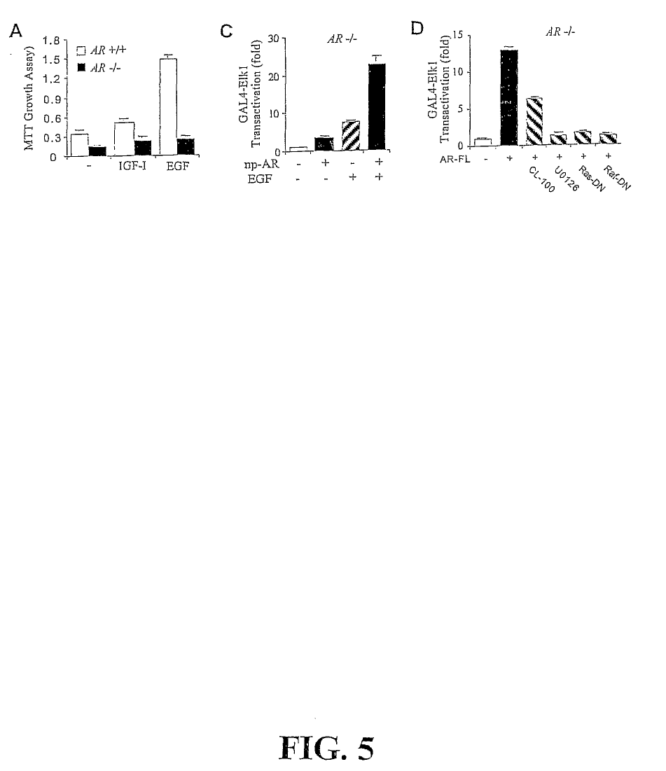

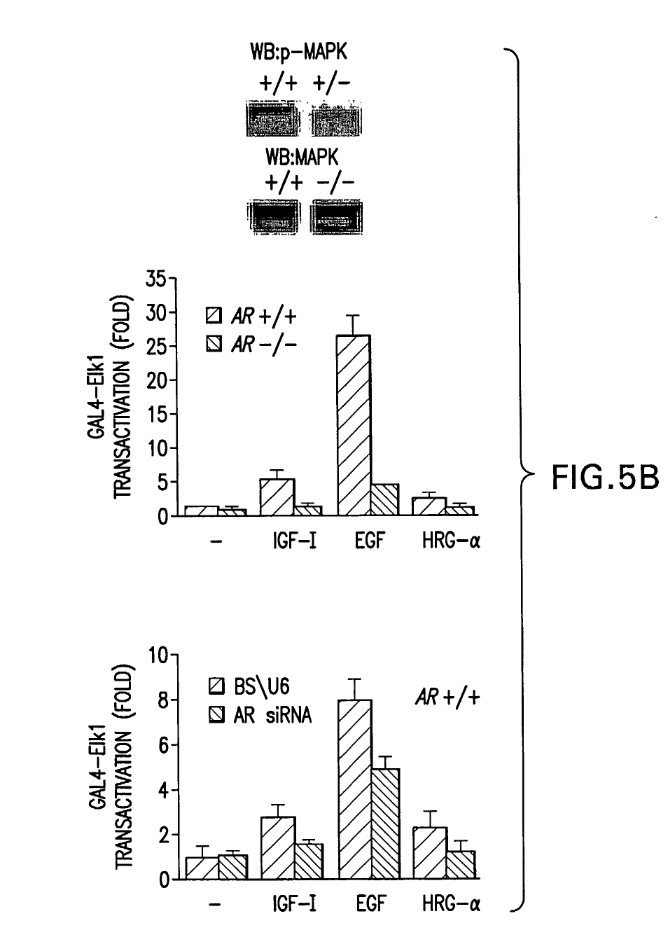

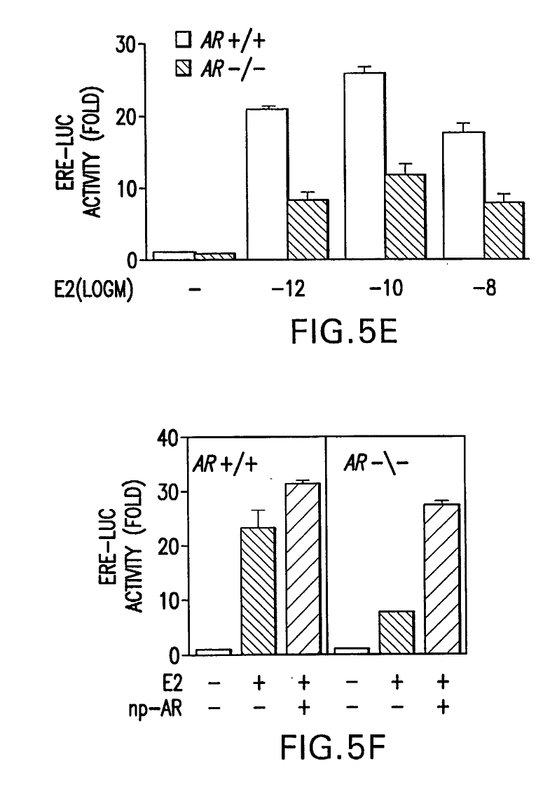

To dissect how AR influences MAPK activity, it was found that loss of AR could disrupt the IGF-I-, EGF-, and HRG-α-induced MAPK activity (FIG. 4B) and reduced IGF-IR expression in AR^{-/-} mice (FIG. 3C). Bonnette et al (Bonnette and Hadsell 2001) found that mice with defective IGF-IR have less branching and decreased cellular proliferation of TEB in developing mammary glands, and these defects could be only partly restored during pregnancy. Similar phenomena also occurred in AR^{-/-} mice (FIGS. 1 and 2), indicating that the suppression of IGF-IR by the AR abrogation can contribute to the retarded mammary gland development in AR^{-/-} mice. Consistent with the data from FIGS. 4F and 5B showing that the loss of AR interrupts HRG-α-induced anchorage-independent cell growth and MAPK activation, Watson et al (Watson et al. 2002) found that in transgenic rats with overexpression of HER2/Neu in the mammary gland, only normal males, but not castrated males, developed mammary tumors. These results may suggest the potential cross-talk between androgen/AR and HER2/Neu signaling pathways in mammary tumor progression.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

### B. COMPOSITIONS AND METHODS

Disclosed are methods of screening a subject for breast cancer comprising: a) obtaining a tissue sample, and b) assaying for the presence of androgen receptor, wherein the presence of androgen receptor indicates an increased risk of or presence of breast cancer. Also disclosed are methods of testing. Screening means identifying the presence of a property while testing means determining if a particular property exists.

Disclosed are methods, wherein the screening is in a cell, wherein the subject is a mouse, wherein the subject is a human, or wherein the subject is male.

Also disclosed are methods of screening a subject for breast cancer comprising: a) obtaining a tissue sample, and b) assaying for the presence of androgen receptor mRNA, wherein the presence of androgen receptor indicates an increased risk of or presence of breast cancer.

Also disclosed are methods, wherein the screening is in a cell, wherein the subject is a mouse, wherein the subject is a human, or wherein the subject is male.

Disclosed are methods of treating cancer comprising administering to a subject an androgen receptor inhibitor.

Also disclosed are methods, wherein the androgen receptor inhibitor reduces nuclear translocation of androgen receptor, wherein the androgen receptor inhibitor comprises ARA67, or fragment thereof, wherein the androgen receptor inhibitor phosphorylates androgen receptor, wherein the androgen receptor inhibitor comprises GSK2B or fragment thereof, wherein the androgen receptor inhibitor reduces an interaction between the N-terminus and C terminus of androgen receptor, wherein the androgen receptor inhibitor comprises hRad9 or fragment thereof, wherein the androgen receptor inhibitor is ARA67, GSK2B, or hRad9, or fragment thereof, wherein the androgen receptor inhibitor interacts with androgen receptor mRNA, wherein the androgen receptor inhibitor is a functional nucleic acid, wherein the androgen receptor inhibitor is an siRNA, wherein the siRNA comprises SEQ ID NO:11, wherein the cancer is breast cancer, or wherein the subject is a male.

Disclosed are methods of screening a composition for the ability to modulate AR activity comprising administering the compound to a system, wherein the system comprises AR and ARA67, GSK2B, or hRad9, and determining if the compound reduces the interaction between AR and ARA67, GSK2B, or hRad9.

A system can be anything having the components necessary to perform the function(s). For example a cell can be system, as well as a test tube, fore example, having the particular components needed for the system to function as needed.

Disclosed are methods of screening a composition for the ability to modulate AR activity comprising administering the compound to a system, wherein the system comprises AR and determining if the compound decreases the amount of nuclear AR.

Disclosed are methods of screening a composition for the ability to modulate AR activity comprising administering the compound to a system, wherein the system comprises AR and determining if the compound decreases the amount of phoshorylated AR.

Disclosed are methods of screening a composition for the ability to modulate AR activity comprising administering the compound to a system, wherein the system comprises AR and determining if the compound decreases the amount of N-terminus Ar interacting with the C-terminus of AR.

Disclosed are methods, wherein the system is a breast cancer cell or cell line or wherein the breast cancer cell line is MCF-7, 7R-75-1, or T47-D.

Disclosed are compositions for inhibiting androgen receptor activity comprising a protein, peptide, antibody, or functional nucleic acid, wherein the composition reduces AR translocation to the nucleus, wherein the composition is not SEQ ID NO:1.

Disclosed are compositions, wherein the composition comprises a fragment of ARA67, wherein the fragment binds androgen receptor.

Disclosed are compositions for inhibiting androgen receptor activity comprising a protein, peptide, antibody, or functional nucleic acid, wherein the composition reduces the interaction between the AR N-terminus and the AR C-terminus, wherein the composition is not SEQ ID NO:7.

Disclosed are compositions wherein the composition comprises a fragment of hRad9, wherein the fragment binds androgen receptor.

Disclosed are compositions for inhibiting androgen receptor activity comprising a functional nucleic acid, wherein the functional nucleic acid interacts with the mRNA of AR.

Disclosed are compositions, wherein the composition comprises an siRNA or wherein the siRNA comprises SEQ ID NO:11.

Disclosed are compositions for inhibiting androgen receptor activity comprising an antibody or a functional nucleic acid, wherein the composition reduces AR translocation to the nucleus, and wherein the composition competes with ARA67 for binding to androgen receptor.

Disclosed are compositions for inhibiting androgen receptor activity comprising an antibody or a functional nucleic acid, wherein the composition reduces AR translocation to the nucleus, and wherein the composition competes with hRad9 for binding to androgen receptor.

Disclosed are compositions for inhibiting androgen receptor activity comprising an antibody or a functional nucleic acid, wherein the composition reduces AR translocation to the nucleus, and wherein the composition competes with GSK2B for binding to androgen receptor.

Disclosed are compositions for inhibiting androgen receptor activity comprising an antibody or a functional nucleic acid, wherein the composition reduces AR translocation to the nucleus, and wherein the composition binds androgen receptor as ARA67 binds androgen receptor.

Disclosed are compositions for inhibiting androgen receptor activity comprising an antibody or a functional nucleic acid, wherein the composition reduces AR translocation to the nucleus, and wherein the composition binds androgen receptor as hRad9 binds androgen receptor.

Disclosed are compositions for inhibiting androgen receptor activity comprising an antibody or a functional nucleic acid, wherein the composition reduces AR translocation to the nucleus, and wherein the composition binds androgen receptor.

Disclosed are compositions, wherein the composition is an antibody, wherein the antibody is a monoclonal antibody, or wherein the antibody is a polyclonal antibody, or wherein the composition is a functional nucleic acid, or wherein the functional nucleic acid is an aptamer.

Disclosed are compounds produced by the method of screening a compound for the ability to modulate AR activity comprising administering the compound to a system, wherein the system comprises AR and ARA67, GSK2B, or hRad9, and determining if the compound reduces the interaction between AR and ARA67, GSK2B, or hRad9 and making the compound.

Disclosed are compounds produced by the method of screening a compound for the ability to modulate AR activity comprising administering the compound to a system, wherein the system comprises AR and determining if the compound decreases the amount of nuclear AR and making the compound.

Disclosed are compounds produced by the method of screening a compound for the ability to modulate AR activity comprising administering the compound to a system, wherein the system comprises AR and determining if the compound decreases the amount of phoshorylated AR and making the compound.

Disclosed are compounds produced by the method of screening a compound for the ability to modulate AR activity comprising administering the compound to a system, wherein the system comprises AR and determining if the compound decreases the amount of N-terminus AR interacting with the C-terminus of AR and making the compound.

Also disclosed are compositions, wherein the composition is not SEQ ID NO1, 5, or 7.

1. AR

Androgen receptor (AR) is a member of steroid hormone receptor (SHR) family and mediates androgen actions that are involved in a wide range of developmental and physiological responses, such as male sexual differentiation, virilization, and male gonadotropin regulation (Quigley, C. A., et al. 1995. Endocr. Rev. 16:271-321, (Brown, T. R., J Androl 16:299-303 (1995)). Besides its physiological roles, AR also contributes to pathological conditions highlighted by its role in prostate carcinogenesis (Quigley, C. A., et al. 1995. Endocr. Rev. 16:271-321, Santen, R. J. 1992. J. Clin. Endocrinol. Metab. 75:685-689). Like other members of SHR family, the AR contains an amino-terminal (N-terminal) transcription activation domain (TAD, amino acids 1-557 SEQ ID NO: 3 are AF1), a DNA-binding domain (DBD, amino acids 557-623), and a carboxyl-terminal ligand-binding domain (LBD, amino acids 624-919). (AF2 aa 872-908) (Mangelsdorf, D. J., et al., Cell 83:835-9 (1995)). Upon ligand binding, the AR dissociates from chaperone proteins including heat shock proteins, homodimerizes, translocates to the nucleus, and turns on the expression of its target genes by binding to the androgen receptor response element (ARE) (Quigley, C. A., et al. 1995. Endocr. Rev. 16:271-321; Chang, C., A. et al., Crit. Rev Eukaryot Gene Expr 5:97-125 (1995)).

a) AR Domains

Compared to the quite conserved DBD and LBD, the N-terminus is quite polymorphic in terms of sequence and length between (nuclear receptors) NRs. The N-terminus is more likely to provide unique surfaces to recruit distinct factors that contribute to the specific action of a certain NR. The AR has a large N-terminus (ARN) and there are two distinct regions important for its transactivation function residing within the ARN: residues 141-338, which are required for full ligand-inducible transactivation, and residues 360-494, where the ligand-independent activation function-1 (AF-1) region is located (Heinlein, C. A., et al. 2002. Endocr. Rev. 23:175-200). Coactivators and corepressors have been identified to interact with ARN (Hsiao, P., et al. 1999. J. Biol. Chem. 274:22373-22379, Hsiao, P., et al. 1999. J. Biol. Chem. 274:20229-20234, Knudsen, K. E., et al. 1999. Cancer Res. 59:2297-2301, Lee, D. K., et al. 2000. J. Biol. Chem. 275:9308-9313, Markus, S. M., et al. 2002. Mol. Biol. Cell 13:670-682, Petre, C. E., et al. 2002. J. Biol. Chem. 277:2207-2215). Furthermore, although ARN extends to more than one half of the fall length protein, its associated proteins are relatively fewer compared to those associated with AR DBD and AR LBD, presumably due to the existence of the AF-1 region which limits the application of conventional yeast-two hybrid system by using ARN as bait. It's likely there are still more ARN associated proteins remaining to be identified.

AR is classified with glucocorticoid receptor (GR), mineralocorticoid receptor and progesterone receptor (PR) as one group within the nuclear receptor (NR) superfamily, since they share high homology in the DBD and recognize very similar hormone response elements (Forman, B. M. et al. 1990. Mol. Endocrinol. 4:1293-1301, Laudet, V., et al. 1992. EMBO J. 11:1003-1013). However, the physiological responses mediated by these receptors upon cognate ligand activation are quite distinct and hormone specific. Apparently, these cannot be explained by a specific DNA-binding through the DBD. Factors located outside the DBD may play a key role in determining the specific hormone responses.

2. Coregulators Interact with AR and Other Steroid Receptors

Steroid receptors may function through direct or indirect interaction with other regulatory proteins in cells (McKenna, N. J., and B. W. O'Malley, Cell 108:465-74 (2002); McKenna, N. J., and B. W. O'Malley, Endocrinology 143:2461-5 (2002)). A number of transcriptional coregulators, including coactivators and corepressors, have been identified that enhance or suppress the interactions between steroid receptors and the basal transcriptional machinery (Hermanson, O., et al., Trends Endocrinol Metab 13: 55-60 (2002); 31. Jepsen, K., et al., Cell 102:753-63 (2000); McInerney, E. M., et al., Proc Natl Acad Sci USA 93:10069-73 (1996); Xu, L., et al., Curr Opin Genet Dev 9:140-7 (1999)). It has been suggested that regulation by coregulators is an efficient way to achieve cell- and promoter-specific activation (Pearce, D. et al. 1993. Science 259:1161-1164). A large number of coregulators have been identified in recent years (reviewed in Heinlein, C. A., et al. 2002. Endocr. Rev. 23:175-200, McKenna, N. J., et al. 1999. Endocr. Rev. 20:321-344). For example, SRC-1 can serve as a coactivator to many NRs like PR, estrogen receptor (ER), GR, thyroid hormone receptor (TR) and retinoid X receptor (RXR) (Onate, S. A., et al., Science 270:1354-1357 (1995)). Although NCo-R and SMRT were initially identified to mediate active suppression by unliganded TR and retinoid acid receptor (Chen, J. D., et al. 1995. Nature 377:454-457, Horlein, A. J., et al. 1995. Nature 377:397-404), later studies suggest that they also serve as corepressors to PR (Wagner, B. L., et al. 1998. Mol. Cell. Biol. 18: 1369-1378), ER (Lavinsky, R. M., et al. 1998. Proc. Natl. Acad. Sci. USA 95:2920-2925) and AR (Dotzlaw, H., et al. 2002. Mol. Endocrinol. 16:661-673, Liao, G., et al. 2003. J. Biol. Chem. 278:5052-5061). It is assumed coregulators that can preferentially bind and influence an individual NR at a specific subcellular environment may help to determine the specificity of NR mediated responses.

The p160/steroid receptor coactivator (SRC) family is the most clearly defined class of coactivators, including SRC-1, SRC-2/TIF2, and SRC-3/AIB1/pCIP/RAC3 (Glass, C. K., and M. G. Rosenfeld, Genes Dev 14:121-41 (2000); Llopis, J., et al., Proc Natl Acad Sci USA 97:4363-8 (2000); McKenna, N. J., and B. W. O'Malley, Cell 108:465-74 (2002)). Interaction between ligand-activated steroid receptors and the p160 coactivators is mediated by a small ˜t-helical motif containing the LXXLL sequence (where L is leucine and X is any amino acid) (44). Ligand binding leads to realignment of the helix 12 in the LBD domain revealing a hydrophobic groove where the LXXLL motifs bind (Bledsoe, R. K., et al., Cell 110: 93-105 (2002), Darimont, B. D., et al., Genes Dev 12:3343-56 (1998), Feng, W., et al., Science 280:1747-9 (1998), Heery, D. M., et al., Nature 387:733-6 (1997)). In addition to LXXLL motifs, a number of AR coregulators, such as ARA54 and ARA70, interact with AR in an androgen-dependent manner through FXXLF motifs (where F is phenylalanine) (He, B., et al., J Biol Chem 277:10226-35 (2002), Kang, H. Y., et al., J Biol Chem 274:8570-6 (1999), 63. Yeh, S., and C. Chang, Proc Natl Acad Sci U S A 93:5517-21 (1996)). Furthermore, the FXXLF motif located in the AR N-terminal region is found to mediate the interaction between the LBD and N-terminus of AR (N/C interaction), which is important for the full AR transactivation capacity (Chang, C., J. D. et al., Mol Cell Biol 19:8226-39 (1999), He, B., et al., J Biol Chem 275:22986-94 (2000), Langley, E., et al., J Biol Chem 270:29983-90 (1995)). Phage display technique confirms the FXXLF motif is a ligand-dependent AR associated peptide moti (Hsu, C. L., et al., J Biol Chem 278:23691-8 (2003)).

3. Prostate Cancer

Prostate cancer is the most common invasive malignancy and second leading cause of cancer deaths in males in the United States (Gittes, R. F. (1991) NEnglJMed324 (4), 236-45, Greenlee, R. T., et al. (2000) CA Cancer J Clin 50 (1), 7-33). In the early stages of this disease, most patients respond favorably to androgen ablation and antiandrogen therapy. Unfortunately, the effects of androgen ablation are usually transient as cancer cells eventually progress into the androgen-independent phenotype. Although the mechanism underlying this resistance to androgen ablation remains largely unknown, mutations in the androgen receptor (AR), enhanced expression of growth factor receptors and associated ligands, and overexpression of some AR cofactors have been shown to be the causal genetic events in prostate cancer progression (Gittes, R. F. (1991) NEnglJMed324 (4), 236-45).

4. Androgen Receptor Signaling

Androgen exerts its effects via the intracellular AR, a member of the superfamily of nuclear receptors (Chang, C. S., et al. (1988) Science 240 (4850), 324-6, Mangelsdorf, D. J., et al. (1995) Cell 83 (6), 835-9). Upon androgen binding, AR dissociates from the heat-shock proteins and binds to androgen response elements (AREs), resulting in upregulation or downregulation of the transcription of AR target genes. In addition to responding to ligands, the AR is affected by kinase signaling pathways which directly or indirectly alter the biological response to androgens. This phenomenon is mediated by the AR, as antiandrogens have been shown to block kinase-induced transcriptional activation (Sadar, M. D. (1999) JBiol Chem 274 (12), 7777-83). Growth factors, cytokines, and neuropeptides have been implicated in various in vitro and in vivo models of human malignancies, including prostate cancers (Burfeind, P., et al. (1996) Proc Natl Acad Sci USA 93 (14), 7263-8). In the absence of androgens, insulin-like growth factor-1 (IGF-1), keratinocyte growth factor (KGF), and epidermal growth factor (EGF) are able to activate transcription of androgen receptor-regulated genes in prostate cancer cells (Culig, Z., et al. (1995) Eur Urol 27 (Suppl 2), 45-7). MAPK and Akt kinase cascades have been shown to be involved in growth factor-mediated AR activation (Yeh, S., et al. (1999) Proc Natl Acad Sci USA 96 (10), 5458-63, Wen, Y., et al. (2000) Cancer Res 60 (24), 6841-5, Lin, H. K., et al. (2001) Proc Natl A cad Sci USA 98 (13), 7200-5). Some neuropeptides, such as bombesin and neurotensin, can stimulate AR activation and cancer cell growth in the absence of androgen, by activation of tyrosine kinase signaling pathways (Lee, L. F., et al. (2001) Mol Cell Biol 21 (24), 8385-97). Prostate cancer cells may progress from androgen-dependence to a refractory state resulting from activation of AR by various kinases, thus circumventing the normal growth inhibition caused by androgen ablation.

5. AR Role in Normal Mammary Cell Development

Epidemiological studies indicated some positive correlation between testosterone concentration and breast cancer incidence, although it is arguable that testosterone effects on breast cancer progression could also come from conversion to 17β-estradiol (E2) via aromatization in peripheral tissues (Secreto and Zumoff 1994; Berrino et al. 1996). Other reports, however, also suggested that androgens could negatively regulate the growth of mammary epithelial and breast cancer cells (Birrell et al. 1995; Szelei et al. 1997; Dimitrakakis et al. 2002). AR is expressed in normal breast and up to 85% of breast tumors are AR-positive (Lea et al. 1989; Kuenen-Boumeester et al. 1992; Wilson and McPhaul 1996). Also, 25% to 82% of metastatic breast tumors, which are ER- and PR-negative, still express a significant amount of AR (Lea et al. 1989; Bayer-Garner and Smoller 2000). Disclosed herein, AR itself, not androgen mediated AR activity, is responsible for normal mammary gland development and is involved in mammary cancer. Thus, disclosed are 1) methods of diagnosing breast cancer based on the presence of AR, and 2) methods and compositions for inhibiting breast cancer wherein the compositions inhibit AR activity, including AR activity that is androgen independent.

Female mice which are homozygous knockouts of AR have smaller mammary glands, ovaries, and uterus than normal female mice. The weight of these organs are 15-23% less in female AR^{-/-} mice as comparing to their age-matched littermates. Disclosed herein, there is a role for AR in the normal development of breast tissue in mice, and this role is involves the MAPK and IGF-I and IGF-I receptor (IGF-IR) pathways.

Specifically, the loss of AR causes a reduction in the number and size of the terminal bud ends, which is related to a reduction in the number of mammary glands. Furthermore, the size and number of cap cells, which are responsible for the ductal extension from the terminal end buds were reduced. On the whole, the mammary glands were less functional in mice lacking AR, having less milk production. A full discussion of the defects of mammary gland development in mice lacking AR can be found in the Examples.

Disclosed herein, the defects in mammary gland development, caused by the loss of AR in the female mice, is linked to the signaling pathways of MAPK and IGF-I/IGF-IR. Phospho-MAPK activity was decreased in the AR−/− mice, even though total MAPK protein remains about the same. IGF-I and IGF-IR are upstream regulators of MAPK. It was found that IGF-IR, but not IGF-I, mRNA expression is reduced by 46% in immature female AR^{-/-} mice (FIG. 3C) consistent with the IGF-I/IGF-IR→MAPK signaling pathway being defective in female AR^{-/-} mice. Cyclin D1 is a down stream target in the IGF-I/IGF-IR/MAPK pathway. The cyclin D1 mRNA expression was significantly reduced in female AR^{-/-} mice (FIG. 3C). Similar reduction of the cyclin D1 protein levels, using immunostaining, also occurred.

The data disclosed herein indicates that AR plays a role in upregulating the signaling of the IGF-I/IGF-IR→MAPK→cyclin D1 pathway through upregulation of the IGF-IR. Thus, a downregulation or a loss of AR will cause a down regulation or loss of signaling through the IGF-I/IGF-IR→MAPK→cyclin D1 pathway because of a down regulation of IGF-IR, and this down regulation will result in retarded and defective mammary gland development in female mice.

Thus, disclosed are methods of regulating the IGF-F/IGF-IR→MAPK→cyclin D1 pathway through the regulation of the amount of active AR, by for example, regulating the amount of AR or its activity, as AR is a positive regulator of this pathway. In addition, this AR effect occurs at least in the prepuberty stage of development, i.e. in early mammary gland development. Thus, the role of growth factors, such as IGF-I are modulated by the presence or absence of AR, which modulates the presence of the IGF-I receptor. This regulation can be accomplished using any of the means of regulation of AR known and/or explicitly disclosed herein.

In addition, the AR−/− mice had reduced signaling from the Estrogen receptor (ER), as estrogen responsive genes, Efp and hepatocyte growth factor (HGF) were down regulated in prepuberty female mice lacking AR. However, Progesterone Receptor (PR) expression was normal in prepuberty female AR^{-/-} mice, but progesterone was decreased in adult mice.

6. AR Role in Breast Cancer

The role of AR in breast cancer was investigated by taking a breast cancer cell line, an MCF7 cell line, and making an MCF7 line that was lacking AR, through homologous recombination and another set of knockdown AR through siRNA for AR. The proliferation of MCF7 cells lacking AR was severely reduced when cultured in media containing normal, steroid deprived, or 10⁻¹⁰ M E2-treated serum (FIG. 4E). Furthermore, the colony formation was defective, even in response to E2 (10⁻¹⁰M) or heregulin-α (HRG-α, 100 ng/ml), an activator for the HER2/HER3/HER4 family. This data indicate that AR plays an essential role in the development of breast cancer. Thus, disclosed are assays for diagnosing breast cancer and determining the prognosis of a breast cancer patient by assaying the levels of AR in the breast cancer or cells of the breast cancer subject. Also disclosed are methods of modulating breast cancer by reducing the amount of AR activity in the breast cancer cell. For example, disclosed herein are siRNAs that effectively reduce the AR activity in MCF7 breast cancer cells and thus, reduce the tumorgenicity of the breast cancer cells, by for example, reducing the ability of the cells to form colonies in a colony forming assay, or reducing the proliferation of the MCF7 cells.

Furthermore, IGF-I, epidermal growth factor (EGF), or HRG-α are stimulators of MCF7 proliferation through MAPK. However, in cells lacking AR, MAPK activity was impaired and cell proliferation reduced. Furthermore, using siRNA, the IGF-I/EGF/HRG-α-induced MAPK activation (FIG. 5B) and cell proliferation, as judged by cells entering S phase, were also reduced. These effects could be rescued by transfection of an AR expression plasmid under the control of a natural AR promoter (np-AR). Adding EGF with np-AR can enhance synergistically the transactivation of GAL4-Elk1 in AR^{-/-} MCF7 cells, compared with the cells treated with EGF alone (FIG. 5C), indicating a significant involvement of AR in the growth factor signaling pathway. Furthermore, the AR-activated GAL4-Elk1 activity can be diminished by MAPK phosphatase-1 (CL-100) or a specific inhibitor U0126, as well as dominant-negative Ras or Raf (Sugimoto et al. 1998) (FIG. 5D). Also, the reduction of MAPK activation by AR siRNA can be recovered by constitutively activated MEK (EK-CA), Ras (Ras-CA), or Raf (Raf-CA), but not by Rac (Sells et al. 1997) (Rac-CA) or PI3K (p110 subunit). These results indicate that AR is an important upstream regulator of the Ras/Raf/MAPK cascade.

The ER activity in AR^{-/-} MCF7 cells was examined. The transcriptional activities of ER were reduced by 58.8%, 53.8%, and 55.0% in AR^{-/-} MCF7 cells in the presence of E2 at 10⁻¹² M, 10⁻¹⁰ M, and 10⁻⁸ M, respectively, using a ERE-luciferase reporter (FIG. 5E). The reduced transcriptional activity of ER in AR^{-/-} MCF7 cells can be restored by transfection of np-AR (FIG. 5F). These results match well the data in FIG. 3C showing ER target gene expression is reduced in AR^{-/-} mouse breasts.

7. AR Activity in General and in Breast Tissue

AR's function as a steroid hormone receptor (SHR) is well documented. Upon binding of its cognate hormone, Androgen, AR dimerizes and is transported into the nucleus where it is able to act on AR specific genes. AR's role in prostate cancer is also well characterized. Androgen ablation therapy, by chemical or physical castration, remains the treatment of choice, but in prostate cancers treated with androgen ablation therapy, using for example, hydroxyflutamide, which is an anti-androgen, blocking productive androgen binding, and thus, decreasing androgen receptor activity, there is typically a refractory period, where the cells become insensitive to the anti-androgen and proliferate in an androgen independent. While there are multiple mechanisms related to this refraction, including mutations in the AR, enhanced expression of growth factor receptors and associated ligands, and overexpression of some AR cofactors, disclosed herein, there is also an underlying androgen independent activity of AR which is involved in, for example, AR's role in breast cancer. This underlying AR independent activity is at least involved through androgen independent activity of AR in the MAPK activation and subsequent pathways. Thus, disclosed are methods of modulating AR activity, independent of modulating androgen or its effects on AR, but rather through targeting the androgen independent activity of AR that is now understood to be at least involved in breast cancer, for example, non-ER/estrogen and/or non-PR/progesterone related breast cancers.

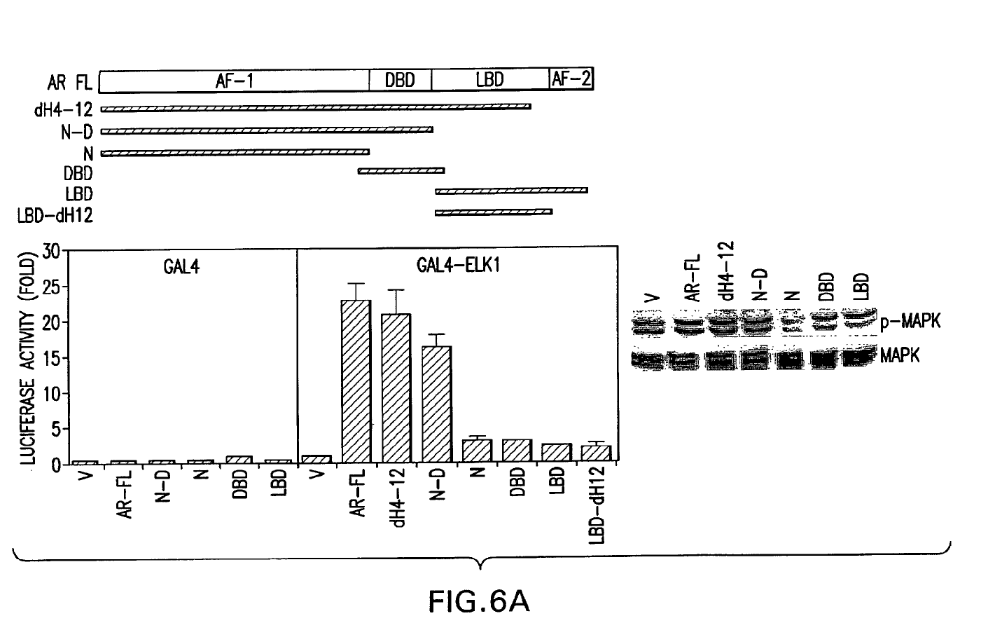

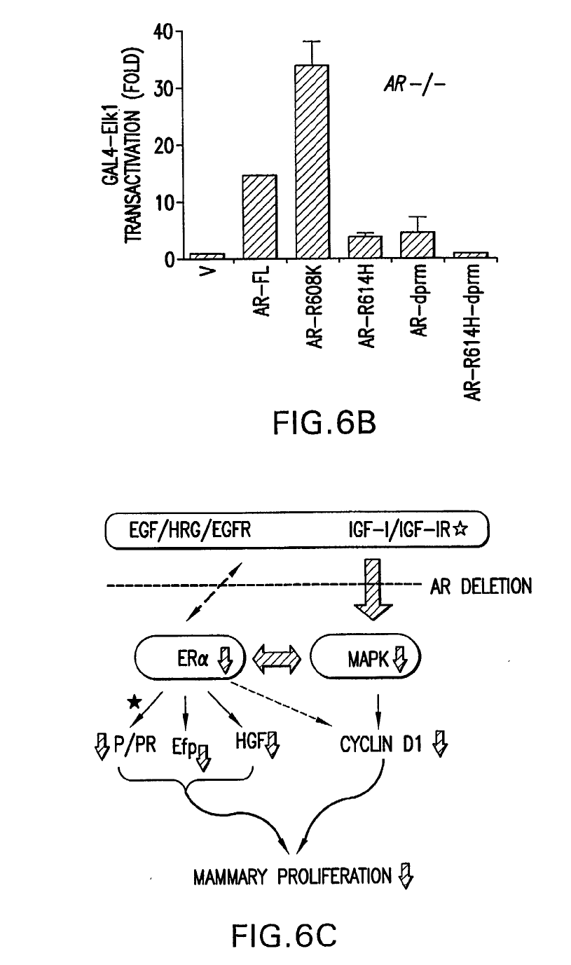

This androgen independent activity was shown herein, by determining that the MAPK activation could be rescued in AR−/− cells with portions of the AR, which lacked the ligand binding domain (LBD). The N-terminus together with DBD, but not LBD, LBD with deletion of helix 12 domain (LBD-dH12), DBD alone, or N-terminus alone, can restore the MAPK activation (FIG. 6A). Thus, androgen receptor, not androgen, is responsible for the activation of the MAPK pathway in breast development and in breast cancer, because androgen receptor lacking the LBD can activate the MAPK pathway.

Disclosed herein, AR is also involved in breast cancer, such as male breast cancer. An AR mutant (AR-R608K) has been suggested to be associated with male breast cancer (Lobaccaro et al. 1993). Disclosed herein in AR^{-/-} MCF7 cells AR-R608K had a higher induction fold on MAPK activation than full length AR (AR-FL) (FIG. 6B), indicating that the contribution of AR-R608K to breast cancer incidence can involve the excessive activation of MAPK.

8. Methods of Inhibiting AR Activity and Inhibiting Cancers Caused by AR Activity

Disclosed are methods of inhibiting AR activity, such as AR activity that is androgen independent, as discussed herein. Typically the methods of inhibiting AR activity involve administering a composition or compound to a cell or organism or in vitro system, such that the compound inhibit activity of the AR, such as the non-androgen dependent activity of AR. Typically, when administering the composition or compound the composition or compound will interact with AR or AR mRNA or other AR nucleic acid, such that, for example the amount of activity AR is decreased (see for example the disclosed siRNA molecules as well as others), the transport of the AR into the nucleus is prevent (See for example, ARA67), the AR is phosphorylated in a region that prevents activity (See for example, GSK3B), of the AR interacts such that interaction between the C and N domains of AR (See for example, hRad9).

It is understood that disclosed herein, there is an interaction between AR and another protein which is required for full AR activity, in for example, breast cancer, where the interaction of AR and the other protein is androgen independent. The methods of inhibiting AR disclosed herein are based on the prevention of this interaction via any of a number of ways, but since the interaction is not dependent on androgen receptor interaction with androgen, antiandrogens, as they have been understood, such as hydroxyflutamide, would not be considered molecules that prevent this non-androgen AR-protein interaction. However, in treating cancers, clearly contemplated would be combination therapies involving antiandrogens, such as hydroxyflutamide, as well as the disclosed AR inhibitors, such as the disclosed AR siRNA molecules or ARA67 or fragments etc.

The compositions can be administered to any animal, including murine, such as mouse and rat and hamster, rabbits, primates, such as chimpanzee, gorilla, orangutan, monkey, or human, ovine, such as sheep and cows, as well as horses.

The disclosed compositions can be used to treat any disease where uncontrolled cellular proliferation occurs such as cancers. Disclosed are methods for regulating cancers related to AR, such as prostate cancer. Disclosed are methods for inhibiting cancers related to androgen receptor. By inhibiting the transactivation activity of AR, such as the non-androgen activity of AR, cancers caused by gene activation or interaction with AR can be reduced.

Disclosed are methods of inhibiting breast cancers comprising administering the disclosed compositions to a cell or an organism or in an in vitro system.

It is also understood that the compositions or compounds can be administered to any type of cell. Typically the compositions and compounds are administered to cells expressing AR and/or AR coregulators, such as co-activators.

Also disclosed are method for diagnosing cancers caused by AR, such as breast cancer. Disclosed herein, the knowledge that there is an androgen independent activity of AR that is involved in cancer, such as breast cancer, indicates that assaying for the presence of AR, independent, for example, to assaying for the presence of androgen, can be predictive of whether the patient has breast cancer. The connection that AR itself is predictive of cancers, such as breast cancer is made herein. Furthermore, the connection between why AR itself and how AR itself is diagnostic of cancers is also disclosed herein. Thus, disclosed are assays designed to determine the presence of AR protein and/or AR mRNA, for example. Any method for determining protein presence, such as ELISA or antibody hybridization or various chromatographic assays can be used to assay for the presence of androgen receptor in samples, such as a cell or tissue, or organisms, such as a human or other animal disclosed herein. Furthermore, any method for assaying nucleic acid presence, such as hybridization technology, such as probe or chip technology, as well as methods involving amplification, such as reverse transcription/PCR can be used to assay for the presence of androgen receptor in a sample, such as a cell or tissue sample or for its presence in an organism, such as a human or other animal disclosed herein.

Disclosed herein, the effect of AR protein can go through interaction with other protein (s) to have non-genomic and/or non-androgenic activities. AR signals can utilize multiple pathways, including the classic androgen/AR→AR target genes of genomic actions as well as AR→AR interaction proteins of non-genomic action to exert its roles in the breast cancer progression. This is in agreement with early reports showing ER could also cross-talk to MAPK in breast cancer cells (Kato et al. 1995; Greene 2003). In addition to estrogens, ER could be activated via phosphorylation at Ser118 by MAPK to induce its target gene expression (Kato et al. 1995). In return, ER could also induce the Ras-Raf-MAPK cascade via non-genomic action (Migliaccio et al. 2000). The results disclosed herein show that AR can influence both MAPK and ER signals therefore indicates that the reduction of ER activity can be due to the reduced MAPK activity and the reduced MAPK activity can be due to the reduced ER activity in AR^{-/-} MCF7 cells and in AR^{-/-} mice.

This study provides the first in vivo evidence showing AR can go through growth factors, MAPK, and ER/PR signals (summary in FIG. 6C) to control the normal breast development, and modulate the breast cancer proliferation, especially in the conditions of absence of or lower E2 (FIG. 4E). Supportively, the epidemiological studies suggest that AR expression is more significantly associated with breast cancer in postmenopausal women than premenopausal women (Lea et al. 1989; Bieche et al. 2001; Honma et al. 2003), and up to the 50% of the AR-positive breast cancers are ER- and/or PR-negative (Bieche et al. 2001; Brys et al. 2002).

9. Molecules Inhibiting AR Activity

Based on the understanding disclosed herein that AR has activity which is androgen independent, for example, not dependent on the LBD, molecules that target the N-terminal domain as well as the DBD are disclosed herein as inhibitors of AR function, for example, in breast cancer. There are a variety of molecules disclosed herein, having the ability to inhibit AR activity which do not target or depend on the androgen related activity of AR. In other words, the disclosed inhibitors of AR activity will inhibit AR independent of androgen effects. For example, the disclosed inhibitors can be used when, for example, AR has become androgen insensitive and antiandrogens, such as hydroxyflutamide do not work because of the refractory state described herein. Thus, the disclosed inhibitors can be used in combination with antiandrogen therapies. Any means for inhibiting AR can be utilized, because as is disclosed herein, there are activities of AR which are androgen independent and for which inhibition of AR itself, is desirable, not just inhibition of the effects of androgen on AR. For example, molecules disclosed in U.S. Pat. No. 6,790,979 by Lee et al., can be used as described herein, which is herein incorporated by reference in its entirety, but at least for molecules that inhibit AR and their structures.

a) Functional Nucleic Acids

Disclosed are functional nucleic acids that interact with either the mRNA, DNA, or proteins, related to AR, ARA67, GSK2B, and hRad9, for example. In certain embodiments the functional nucleic acids can mimic the binding of, for example, ARA67, GSK2B, or hRad9 to AR, and they will bind AR. In other situations, the functional nucleic acids can mimic the binding of AR to ARA67, GSK2B, or hRad9 binding either ARA67, GSK2B, or hRad9.

For example, disclosed are small interfering RNAs that interact with AR nucleic acid, causing a reduction in functional AR, as disclosed herein (See SEQ ID NOsXXX. Small interfering RNA (siRNA) was applied to interrupt AR expression in AR^{+/+} MCF7 cells. It was found that AR siRNA-transfected cells had a lower degree of Ki67 immunostaining, and the mRNA levels of Ki67 and c-myc were reduced by 42% and 81%, respectively. As Ki67 and c-myc are target genes of AR, this indicates that the AR was knocked down. Together, FIG. 4 indicates that AR plays an essential role for the growth of breast cancers.

b) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of any of the proteins disclosed herein, such as ARA67, GSK2B, or hRad9 or the genomic DNA of any of the proteins disclosed herein, such as ARA67, GSK2B, or hRad9 or they can interact with the polypeptide any of the proteins disclosed herein, such as ARA67, GSK2B, or hRad9. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide. For example, when determining the specificity of AR, ARA67, GSK2B, hRad9, for example, aptamers, the background protein could be serum albumin. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J. 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162.

c) Protein and Peptides Inhibiting AR

(1) Nuclear Transport Regulators

(a) ARA67 Functions as a Repressor to Suppress Androgen Receptor Transactivation

In order to identify proteins that are associated with ARN and possibly with AF-1, a new yeast two-hybrid system, the CytoTrap Sos system (Statagene), was employed with fall length ARN as bait, to screen a human prostate cDNA library. One of the clones identified was termed ARA67. Sequence alignment searching revealed that ARA67 matched the sequence encoding protein interacting with amyloid precursor protein tail 1 (PAT1). ARA67/PAT1 (SEQ ID NO:1 protein, and SEQ ID NO:2, cDNA) contains 585 amino acids with a predicted molecular weight of 66.9 kDa. It shares homology with kinesin light chain (Zheng, P. et al. 1998. Proc. Natl. Acad. Sci. USA 95:14745-14750), which is a molecular motor involved in the transportation of cargos along the microtubule. Studies have shown that ARA67/PAT1 can bind microtubules and is involved in amyloid precursor protein (APP) secretion (Zheng, P. et al. 1998. Proc. Natl. Acad. Sci. USA 95:14745-14750).

Disclosed herein ARA67 is an AR interacting protein that functions as a repressor of AR. The mechanism of action of ARA67 is consistent with influencing the nuclear translocation of AR.

ARA 67 was identified by performing a Cytotrap SOS two hybrid experiment with amino acids 1-537 of SEQ ID NO:3, the ARN of AR. ARA67 was shown to bind the ARN specifically, not interacting with TR2, TR4, ARA55, or ARA70 in a yeast two-hybrid assay.

ARA67 interacts with ARN in a DHT independent manner. ARA67 interacts with ARN most strongly, but also interacts with the ARDBD weakly and ARLBD moderately, both in vitro and in vivo.

ARA67 represses DHT dependent AR transactivation as well as coactivator (ARA70N) AR transactivation. ARA67 also repressed prostate specific antigen (PSA) in LNCaP cells, an indication of a repression of AR activity. ARA67 represses AR activity approximately 2.5 times more than PR activity and ER activity was nearly unaffected by ARA67.

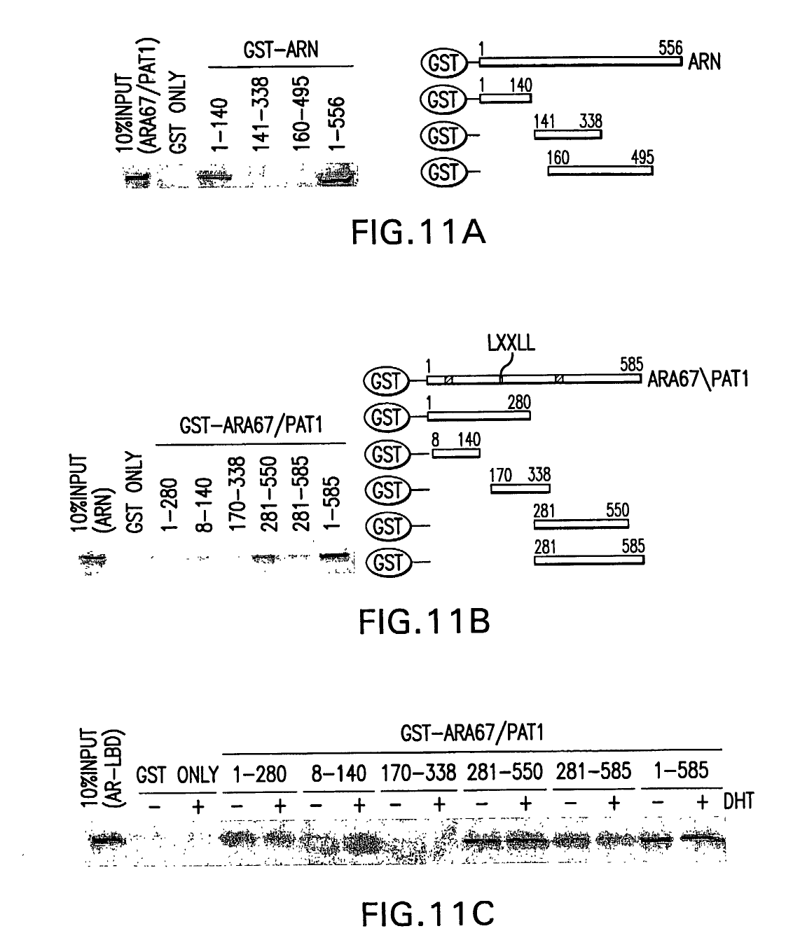

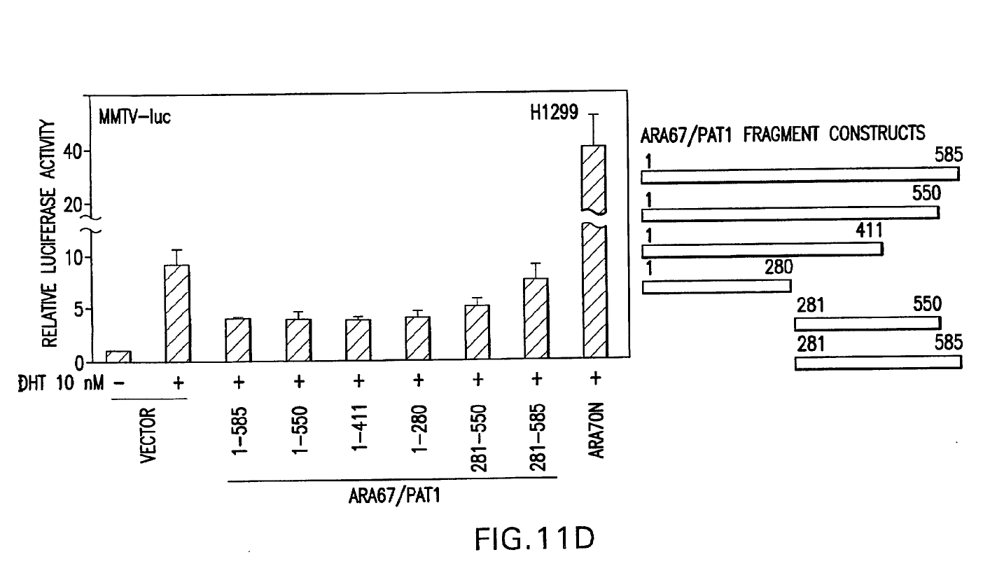

Fragments of ARN interacted with ARA67. ARN₁₋₁₄₀ (SEQ ID NO:3) showed positive interaction although not as strong as that seen in ARN full length (ARN₁₋₅₅₆) (FIG. 11A). These data indicate residues 1-140 within ARN are critical for the interaction with ARA67. Since important regions for AR transactivation within ARN are in residues 141-338, which are required for full ligand-inducible transcription, and residues 360-494, which contain the AF-1 region that is also required for full AR function (Heinlein, C. A., et al. 2002. Endocr. Rev. 23:175-200), the data showing that AR residues 1-140 interact with ARA67 indicate that a new domain within ARN can be involved in ARA67 mediated suppression on AR transactivation.

The N-terminal (ARA67₁₋₂₈₀) and C-terminal (ARA67₂₈₁₋₅₈₅) regions of ARA67 can interact with ARN but the interaction is relatively weak. ARA67₈₋₁₄₀ and ARA67₂₈₁₋₅₅₀ showed slightly stronger interaction with ARN than their bigger counterparts ARA67₁₋₂₈₀ and ARA67₂₈₁₋₅₈₅, respectively, while ARA67₂₈₁₋₅₅₀ was better than ARA67₈₋₁₄₀. Although no fragment constructs of ARA67 strongly interacted with ARN, full length ARA67 showed strong interaction with ARN, indicating participation of different parts of ARA67 can be required for the interaction (FIG. 11B). The interaction pattern between ARA67 fragments and AR LBD was similar to that between ARA67 fragments and ARN, but ARA67 C-terminal fragment showed an interaction strength similar to full length ARA67 (FIG. 11C), which indicates that the interaction between ARA67 and AR LBD may not need the cooperation of the N- and C-termini of ARA67. Amino acid sequences located within 8-140 and 339-550 of ARA67 can contribute more to its interaction with AR (FIGS. 11B, 11C).

(b) ARA67 Fragments

ARA67₁₋₅₅₀, lacking the PEST sequence (lacks the last 35 amino acids) (Gao, Y., et al. 2001. Proc. Natl. Acad. Sci. USA 98:14979-14984) didn't show a stronger suppression effect than full length ARA67. As shown in FIG. 11D, ARA67₁₋₄₁₁ lacking the nuclear localization signal (Gao, Y., et al. 2001. Proc. Natl. Acad. Sci. USA 98:14979-14984) had a similar suppression effect as fall length ARA67 did, indicating that the nuclear localization of ARA67 is not critical for its effect on AR. The N-terminal (ARA67₁₋₂₈₀) and C-terminal (ARA67₂₈₁₋₅₅₀, ARA67₂₈₁₋₅₈₅) regions of ARA67 could also suppress AR transactivation, however ARA67₁₋₂₈₀ was a better suppressor than ARA67₂₈₁₋₅₅₀ and ARA67₂₈₁₋₅₈₅. Together FIG. 11B-D show that both the N- and C-terminal regions of ARA67 are involved in the interaction with and suppression of AR, and the interaction strength is not the sole determinant of suppression potency.

(c) Effect of ARA67 on AR

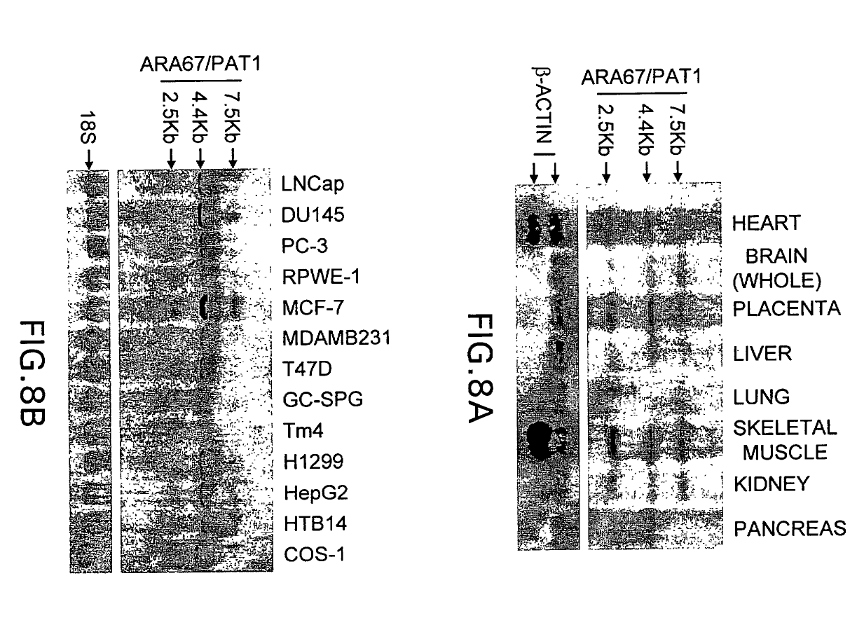

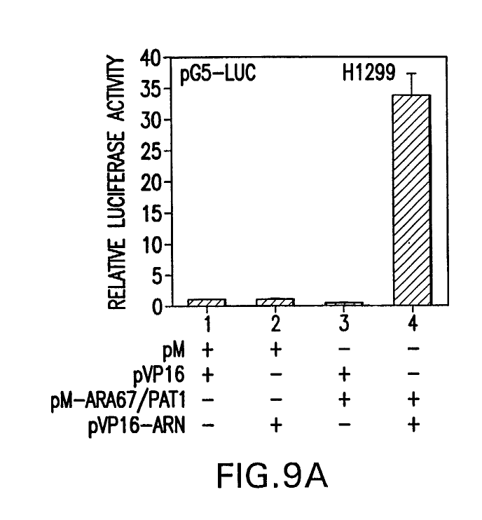

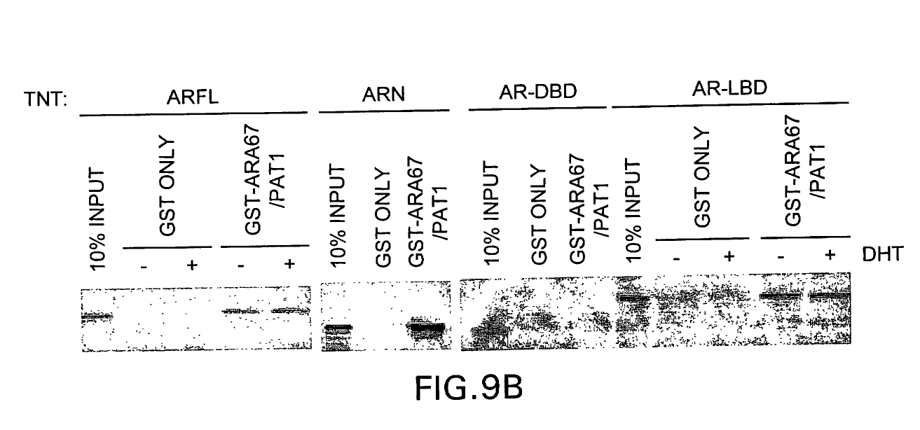

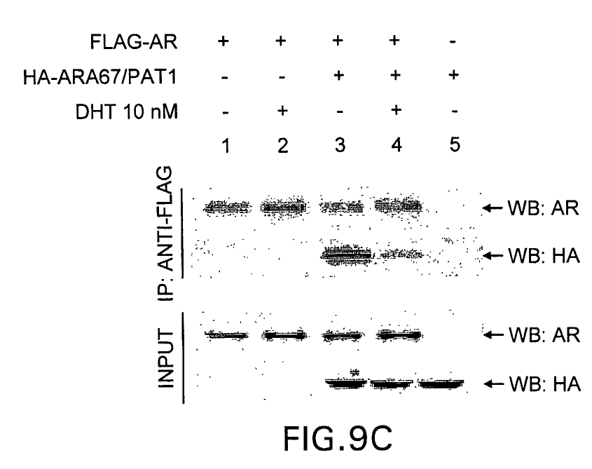

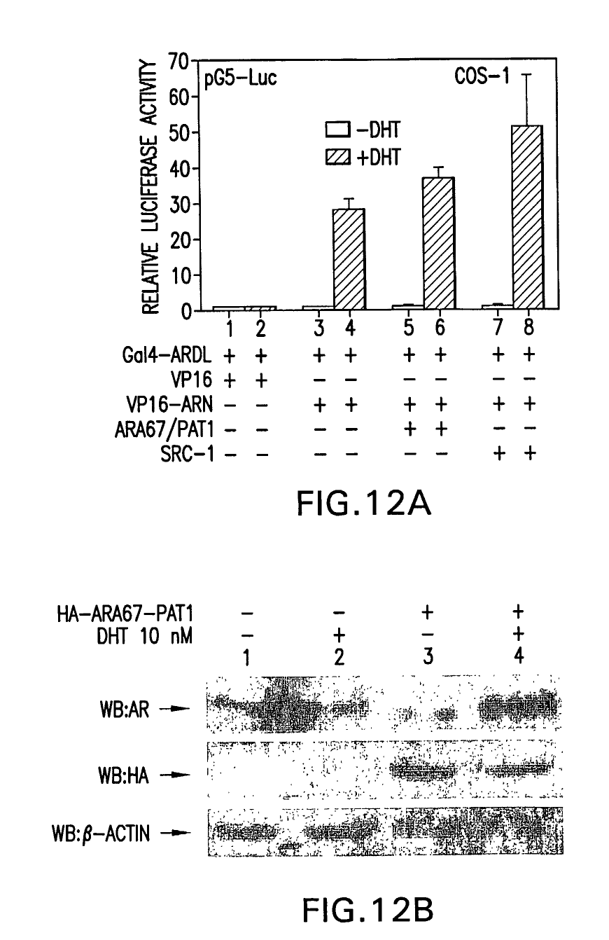

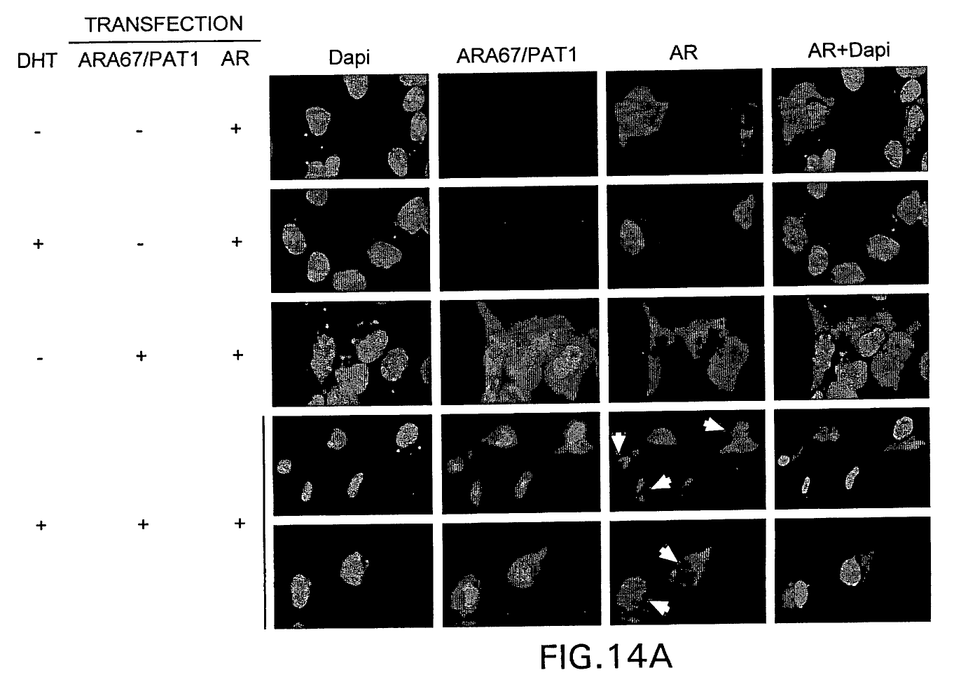

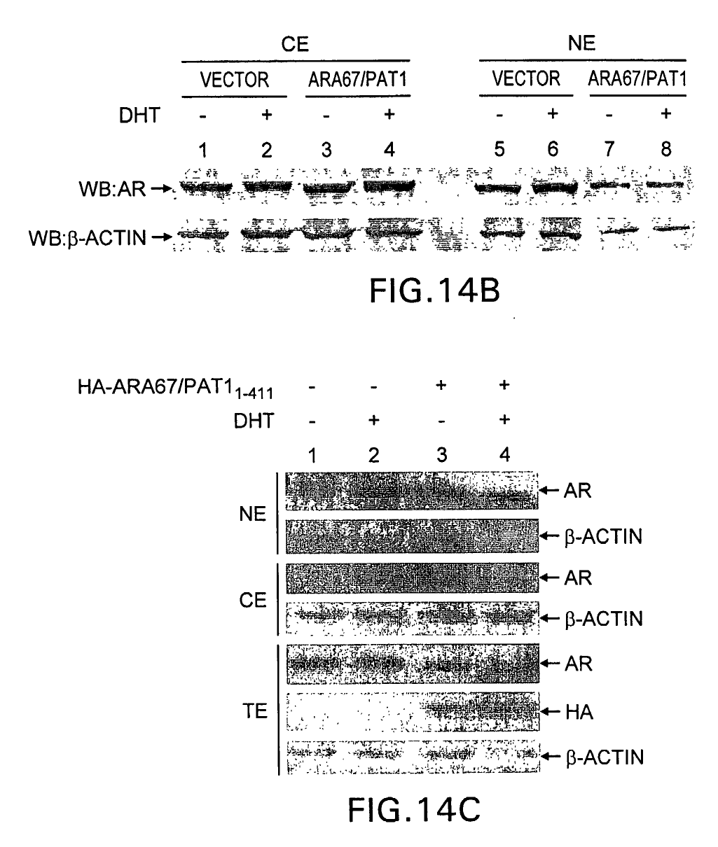

There is an N to C terminal interaction that takes place in AR which stabilizes androgen binding. (Zhou, Z. X., et al. 1995. Mol. Endocrinol. 9: 208-218; Simental J. A., et al. 1991. J. Biol. Chem. 266:510-518). It was shown herein that DHT promoted the AR N/C interaction, and that ARA67 slightly enhanced this association rather than reducing it. (FIG. 9B and FIG. 12A). Furthermore, histone deacetylase (HDAc) is not involved in the ARA67 mediated suppression on AR. Disclosed herein, it is shown that upon DHT binding to AR, AR translocates to the nucleus, but in the presence of ARA67 this translocation is inhibited. (FIG. 14A). Therefore, ARA67 can block the nuclear translocation of AR.

(2) Phosphorylation Regulators

The GSK3β plasmids, including wild type, constitutively active, and dominant negative forms, were kindly provided by J. Sadoshima, Pennsylvania State University.

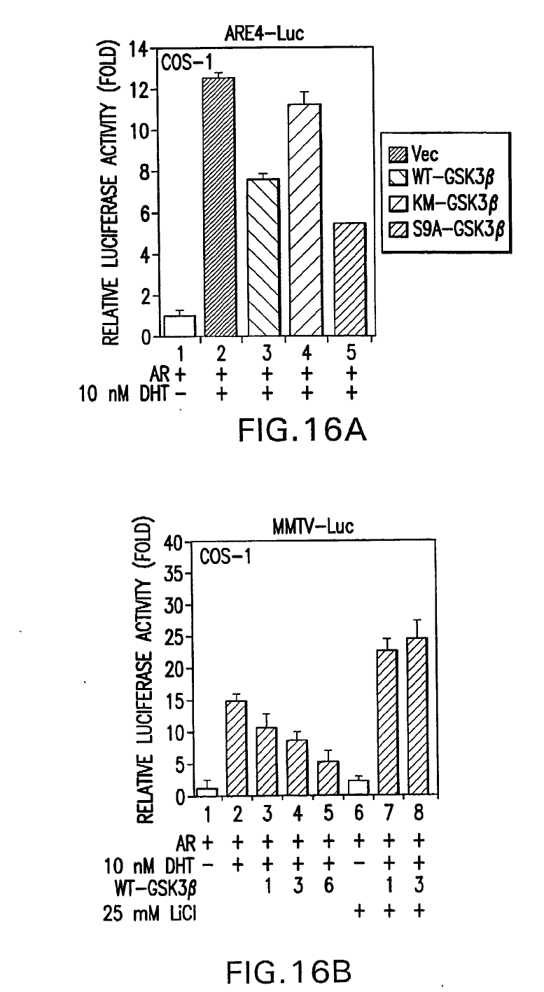

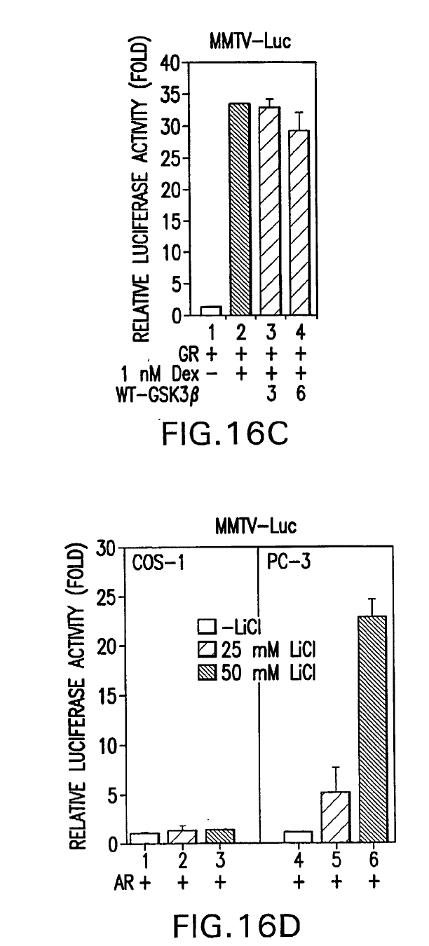

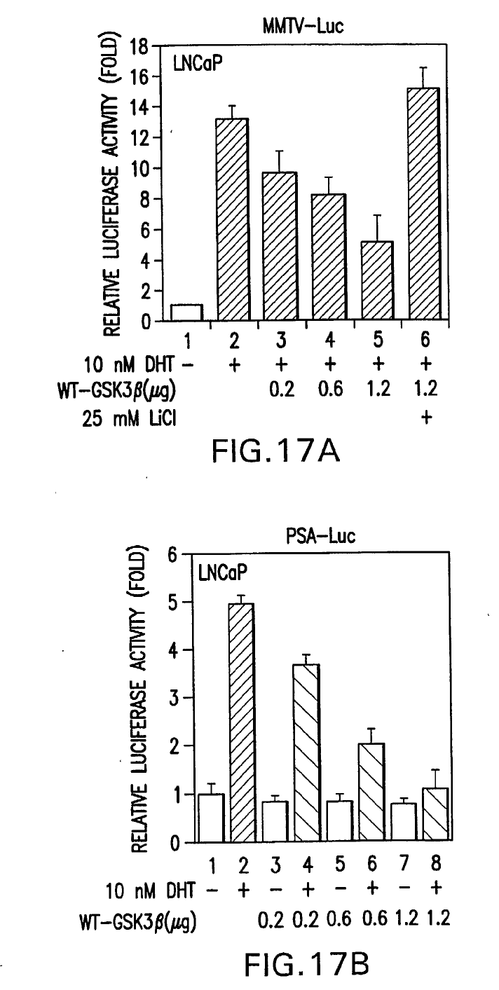

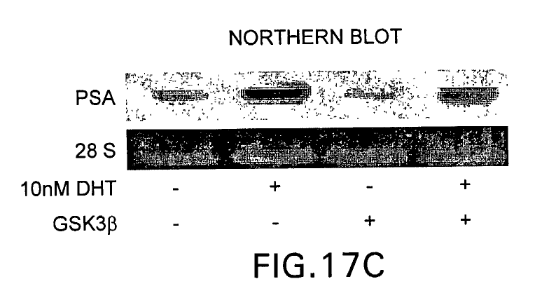

As shown in FIG. 16A, wild type (WT) GSK3β reduced the AR-mediated transcription of the luciferase reporter by about 40% (lanes 2). While inactive GSK3β (KM-GSK3β) had only a maginal effect on AR, the constitutively active form of the GSK3β (S9A-GSK3β) strongly inhibited AR activity (lane 4, and 5), indicating that the kinase activity of GSK3β is necessary to suppress AR activity. FIG. 16B demonstrates that GSK3β inhibits DHT-mediated AR transactivation in a dose-dependent manner (lanes 2-5). Lithium Chloride (LiCl), a specific inhibitor of GSK3β, not only abolished the inhibitory effect of GSK3β on AR, but also slightly enhanced AR transcriptional activity. This result indicates that LiCl can block both exogenously transfected GSK3β as well as the endogenous GSK3β activity in COS-1 cells. The results from FIG. 16A to 16C indicate that GSK3β can selectively inhibit AR transactivation. GSK3β inhibits AR transactivation in LNCaP cells which have mutated yet functional AR. (FIG. 17A). Endogenous PSA protein expression was induced by the treatment of LNCaP cells with DHT. This DHT-mediated induction of transcription from the PSA promoter by DHT was repressed by overexpression of wild type GSK3β (FIG. 17B). The results from Northern blot assays further demonstrated that the expression of PSA mRNA was reduced by the ectopic expression of GSK3β (FIG. 17C). Together, both reporter assay and Northern blot assay indicate that GSK3β inhibits AR transactivation and influences expression of the target gene downstream of the AR.

(a) Glycogen Synthase Kinase 3β (GSK3β)

Glycogen synthase kinase 30 (GSK3β is a serine/threonine protein kinase that was first described in a metabolic pathway for glycogen synthase regulation (Cohen, P., et al. (1978) Biochem Soe Symp 43, 69-95). It is now clear that GSK3β is a multifunctional kinase that regulates a wide range of cellular processes, ranging from intermediate metabolism and gene expression to cell fate determination, and proliferation and survival (Hardt, S. E., et al. (2002) Circ Res 90 (10), 1055-63, Krylova, O., et al. (2000) J Cell Biol 151 (1), 83-94, Harwood, A. J., et al. (1995) Cell 80 (1), 139-48, Wang, Q., et al. (2002) J Biol Chem 24, 24). GSK3β phosphorylates a broad range of substrates, including several transcription factors such as c-myc, c-Jun, rat glucocorticoid receptor, heat-shock factor-1, nuclear factor of activated T-cells c, and β-catenin (Sears, R., et al. (2000) Genes Dev 14 (19), 2501-14, de Groot, R. P., et al. (1993) Oncogene 8 (4), 841-7, Rogatsky, I., et al. (1998) J Biol Chem 273 (23), 14315-21, He, B., et al. (1998) Mol Cell Biol 18 (11), 6624-33, Beals, C. R., et al. (1997) Science 275 (5308), 1930-4, Aberle, H., et al. (1997) Embo J 16 (13), 3797-804). In contrast to other kinases, GSK3β is highly active in unstimulated cells and becomes inactivated in response to mitogenic stimulation (Cohen, P., et al. (2001) Nat Rev Mol Cell Biol 2 (10), 769-76). Growth factors down-regulate GSK3β activity through the PI3K/AKT signaling cascade and the MAPK/p90RSK pathway (Cross, D. A., et al. (1995) Nature 378 (6559), 785-9, Torres, M. A., et al. (1999) Mol Cell Biol 19 (2), 1427-37). Consistent with its position downstream of the PI3K-AKT and MAPK-p90RSK pathways, GSK3β activity suppresses cell proliferation and induces apoptosis (Hoeflich, K. P., et al. (2000) Nature 406 (6791), 86-90, Hall, J. L., et al. (2001) Diabetes 50 (5), 1171-9). Phosphorylation of serine-9 of GSK3β inhibits its activity by creating an inhibitory pseudosubstrate for the enzyme. Conversely, mutations that prevent this phosphorylation result in activation of the kinase. GSK3β is also inhibited by Wnt signaling, which may contribute to progression of the prostate cancer (Chesire, D. R., et al. (2002) Oncogene 21 (17), 2679-94).

Disclosed herein, GSK3β inhibits AR-dependent transactivation of several reporter genes as well as endogenous DHT-mediated PSA expression. Additionally, the data indicate that the effect of GSK3β is mediated through the NH2-terminal activation function (AF-1) of the AR. Moreover, the results indicate that GSK3β can interact directly with the AR and inhibit androgen-stimulated cell growth. These findings indicate that GSK3β can directly modulate AR signaling and, therefore, can play important roles in the control of the proliferation of normal and malignant androgenoregulated tissues.

(b) GSK-3β Phosphorylates the Amino Terminus of AR in Vitro and Inhibits the Function of the Ligand-Independent Activation Domain (AF-1).

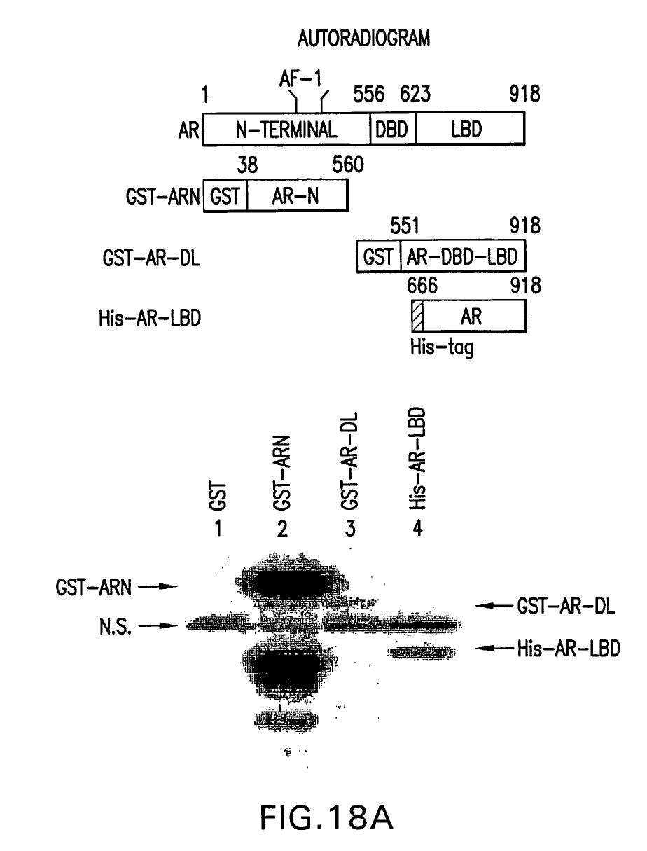

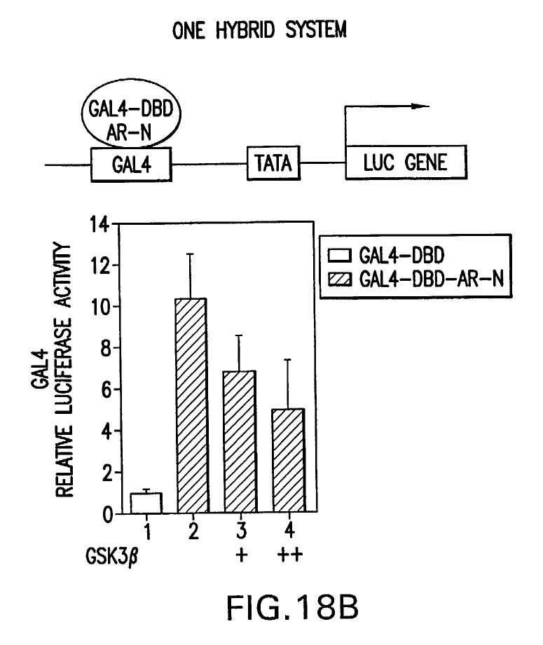

Since the data indicate that GSKβ kinase activity is necessary for inhibiting AR transactivation, the task of determining whether AR is a substrate for GSK3β was undertaken. GSK3β phosphorylates the N-terminal of AR (amino acids 38-560 of SEQ ID NO:3), in the AF-1 region. Addition of wild type GSK3β inhibited the constitutive transcriptional activity of GAL4-ARN. (FIG. 18). In contrast, GSK3β did not influence the activity of GAL4-AR-LBD, which contains the AF-2 domain. These results indicate that GSK3β can suppress AR transactivation via the AF-1 functional domain that is located in the AR N-terminal in vitro. Furthermore, GSK3β can interact with ARN in vitro. As demonstrated in FIG. 19C, GSK3β forms a stable complex with AR, indicating that GSK3β can interact with AR in the same cell and AR could be a substrate for GSK3β in vivo.

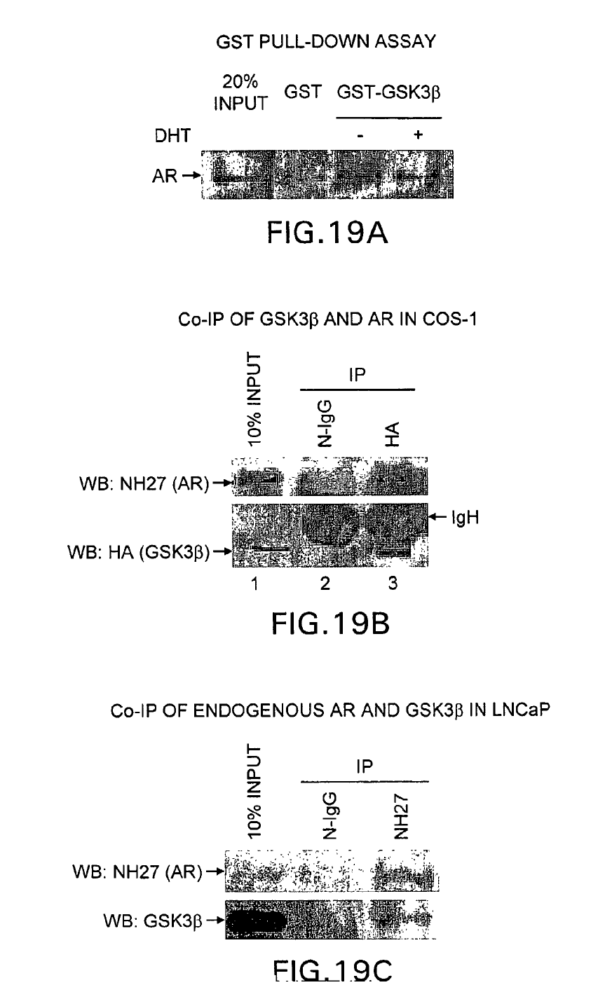

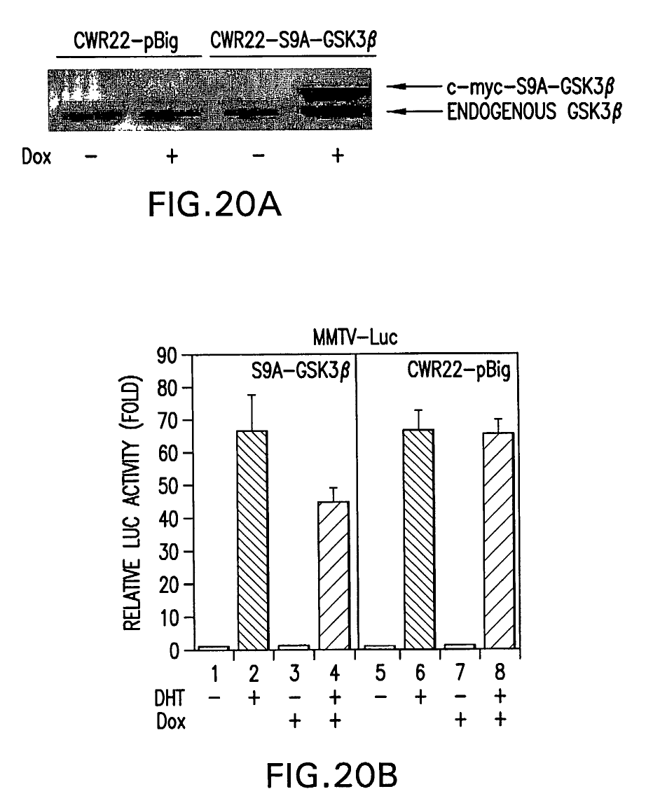

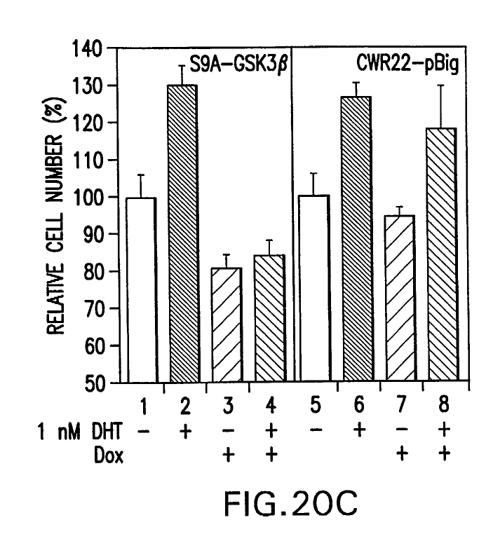

Inducible S9A-GSK3β plasmids were introduced into the androgen-dependent CWR22R cell line by stable transfection. To distinguish exogenously transfected GSK3β from endogenous GSK3 β in CWR22R cells, a myc-tagged S9A-GSK3β was constructed in the pBIG vector. Doxycycline stimulated the S9A-GSK3β expression in CWR22R-S9A-GSK3β cells but not in the vector transfected CWR22R-pBig cells (FIG. 20A). Using a Luc reporter assay, it was found that induction of S9A-GSK3β reduced AR transactivation by 30% while doxycycline had a marginal effect on CWR22R-pBig cells. This effect likely represents an underestimate of the total impact of GSK3β on AR activity since CWR22R cells express endogenous GSK3β. To correlate the inhibitory effect of GSK3β on AR with prostate cancer cell growth, the growth of stable-transfected CWR22R cells was tested in an MTT assay. The MTT assay (FIG. 20C) shows that addition of DHT induced cell growth in both CWR22R-pBig and CWR22R-S9A-GSK3β cells. As expected, the doxycycline treatment caused obvious growth arrest in the CWR22R-S9A-GSK3β cells, but not in the CWR22R-pBig cells. Taken together, these data indicate that activation of GSK3β inhibits AR transcriptional activity and correlates with the reduced cell growth. GSK3β inhibited the interaction of AR with ARA70 (lane 7 vs. 5), indicating that the inhibition of AR transactivation by GSK3β can involve reduced interaction between AR and AR coregulators.

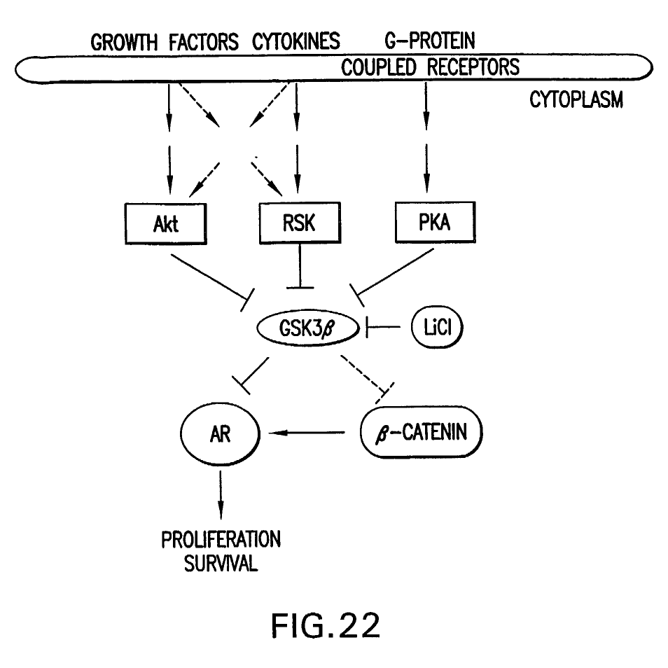

The AR-signaling pathway can be still functional in androgen-refractory cancers. The AR is a phosphorylated protein and its phosphorylation status is associated with its transcriptional activation. The N-terminal of AR contains the majority of the sites phosphorylated in vivo (Kuiper, G. G., et al. (1993) Biochem J291 (Pt 1), 95-101). Alteration of AR phosphorylation by factors with elevated expressions in some prostate cancers may provide one possible mechanism involved in stimulating the progression of prostate cancer. These factors include cytokines, growth factors, and G-protein coupled receptors and their activity often leads to the inactivation of GSK3β. Disclosed herein, GSK3β modulates AR transcriptional activity and phosphorlyates AR. Specifically, forced overexpression of GSK3β inhibits transcription of PSA in LNCaP prostate cancer cells. Overexpression of constitutively active S9A-GSK3β leads to the growth arrest of prostate cancer cells (FIG. 20), thus, the inhibition of GSK3β can contribute to the development and progression of androgen-independent prostate disease. Considering that PKA, Akt, and MAPK inhibit GSK3β (FIG. 22), the data presented here are consistent with what is known regarding the stimulation of prostate cancer cell growth by growth factors and cytokines, and fit very well with the pro-apoptotic roles of GSK3β in other tissues (Hardt, S. E., et al. (2002) Circ Res 90 (10), 1055-63, Culbert, A. A., et al. (2001) FEBSLett 507 (3), 288-94, Pap, M., et al. (1998) J Biol Chem 273 (32), 19929-32). Recent studies also demonstrate that GSK3β may regulate AR activity through β-catenin, an AR coactivator. Disclosed herein GSK3β directly influences AR activity, independent of the β-catenin mediated pathway. The interaction between AR and β-catenin is DHT-dependent, and the data demonstrate that the inhibition of GSK3β by lithium chloride increases AR transcriptional activity in the absence of DHT. GSK3β directly phosphorylates the N-terminal region of AR. The GSTpull-down assay and co-Immunoprecipitation assay indicate the interaction between GSK3β and AR (FIG. 19A).

Disclosed herein AR phosphorylation and the resulting inhibition of AR activity is consistent with the blockage of DHT-induced cell growth imposed by activated GSK3β (FIG. 20). In addition, GSK3β is known to phosphorylate many other molecules including cyclin D1, cJun, and cMyc, which can lead to CDK4 and CDK6 activation these can be involved in prostate cancer proliferation as well. (Sears, R., et al. (2000) Genes Dev 14 (19), 2501-14, Alt, J. R., et al. (2000) Genes Dev 14 (24), 3102-14, Diehl, J. A., et al. (1998) Genes Dev 12 (22), 3499-511; Kokontis, J., et al. (1994) Cancer Res 54 (6), 1566-73, Miyoshi, Y., et al. (2000) Prostate 43 (3), 225-32; Boyle, W. J., et al. (1991) Cell 64 (3), 573-84, Pfahl, M. (1993) Endocr Rev 14 (5), 651-8). Active GSK3β therefore, is implicated as a key factor in maintenance of the basal states of several important signaling pathways, and dysregulation of GSK3β can lead to transformation to malignancy.

Disclosed are molecules that mimic or increase GSK3β activity, and these molecules can be used in the treatment of AR dependent cancers. For example, molecules that bind AR in a way similar to the way GSK2B binds AR can have similar inhibition activities of AR.

(3) Inhibitors of the AR N/C Interaction

It's known that AR N- and C-terminus can directly interact through the LXXLL like motif present in AR N-terminus and AF-2 domain in AR C-terminus (He, B., et al. 1999. J. Biol. Chem. 274:37219-37225, He, B., et al. 2000. J. Biol. Chem. 275:22986-22994). Upon ligand binding, helix 12 in AR LBD folds across the ligand binding pocket, which reduces the dissociation rate of bound androgen and helps to stabilize AR protein. AR N/C interaction stabilizes the position of helix 12 when androgen is bound to AR (Zhou, Z. X., et al. 1995. Mol. Endocrinol. 9: 208-218, He, B., et al. 1999. J. Biol. Chem. 274:37219-37225). Coregulators that influence the AR N/C interaction could affect the stability of AR and thus AR transactivation.

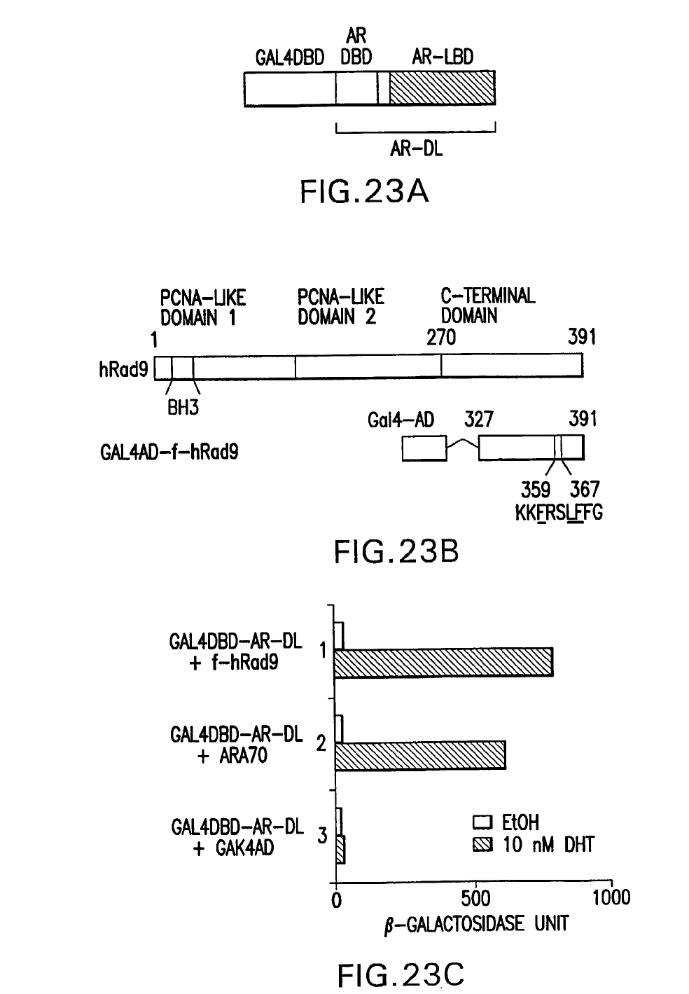

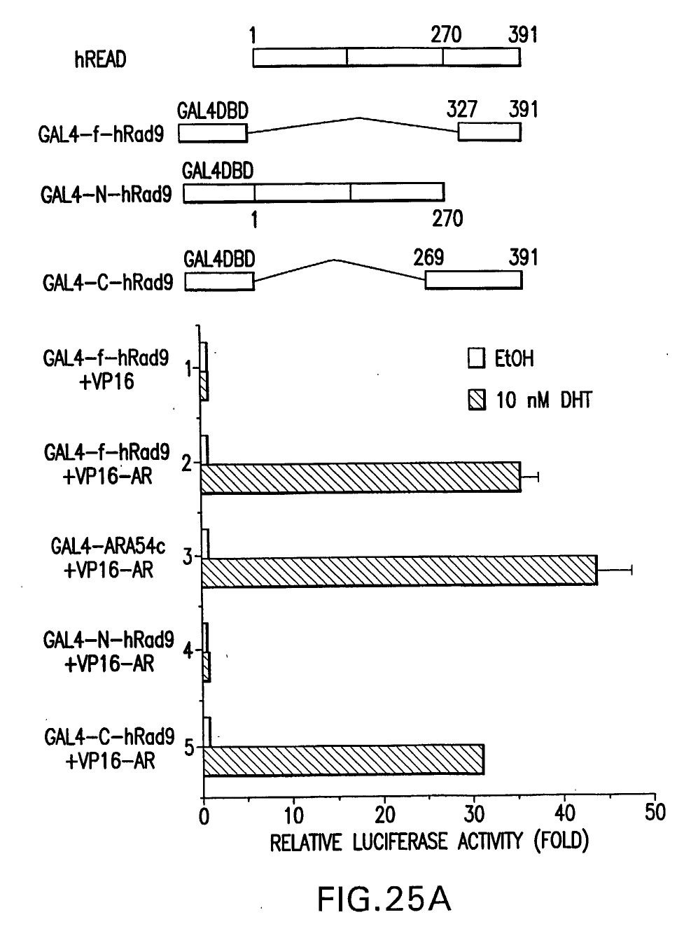

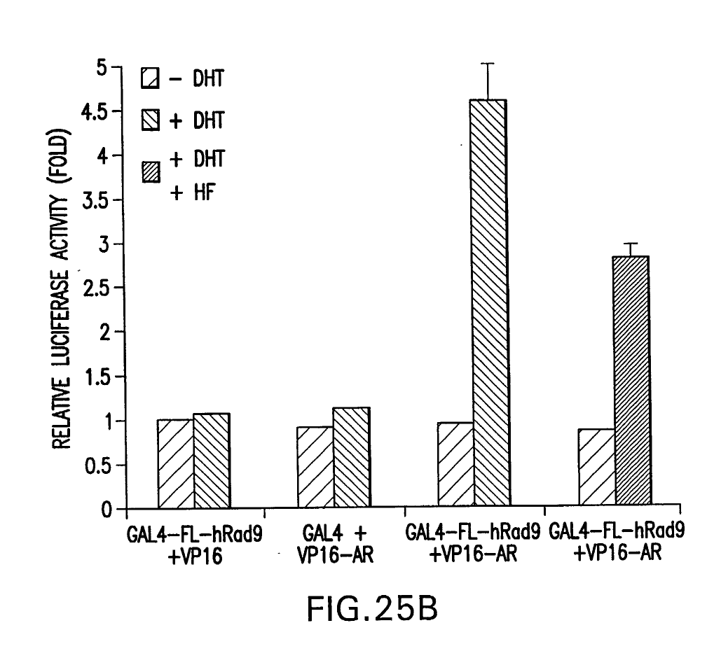

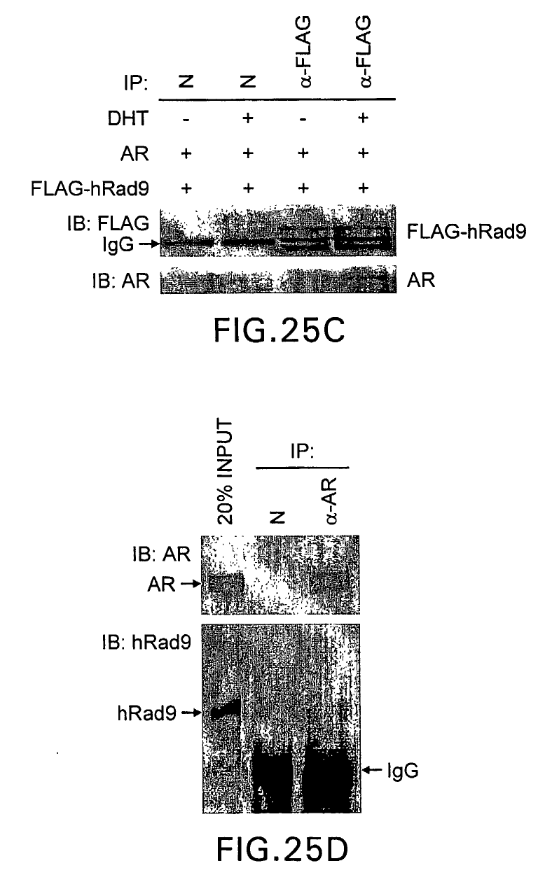

In a yeast two-hybrid screen designed to identify ligand-dependent interaction with AR a fragment of hRad9 was identified. This fragment was amino acids 327-391 of SEQ ID NO:7 and interacted with the AR DBD-LBD. The hRad9 fragment from yeast lies in the C-terminus of hRad9 and contains all FXXLF (aa.361-365) motif that overlaps with the potential nuclear localization sequence (NLS) motif (aa.356-364) (Hirai, I., and H. G. Wang, J Biol Chem 277:25722-7 (2002)). This fragment of hRad9 is referred to as f-hRad9 (FIG. 23B). Disclosed herein is the androgen-dependent interaction between AR and hRad9 in yeast. The C-terminus of hRad9 (aa 269-391) displayed a strong interaction with AR in the presence of androgen while the PCNA-like domain of hRad9 (N-hRad9, aa 1-270) did not (FIG. 25A, lane 5 and 4, respectively), indicating the C-terminus of hRad9 mediates the interaction with AR. Full length hRad9 (FL-hRad9) was stimulated to interact with AR in the presence of androgen and was inhibited by the addition of hydroxyflutamide (HF), an antagonist for AR. The interaction between hRad9 and AR also occurs in mammalian cells.

In human prostate cancer samples quantitative real time PCR indicated that hRad9 expression is reduced in neoplastic samples as compared to normal samples, in some cases. This is consistent with hRad9 being down regulated in prostate cancers and in a subset of prostate cancers.

(a) Rad Family of Proteins

Unrepaired DNA lesions, arising from either intrinsic or exogenous sources, lead to genomic instability and consequently contribute to the development of cancers (Hartwell, L. H., and M. B. Kastan, Science 266:1821-8 (1994)). Cell cycle checkpoints and DNA repair are the primary defenses against genomic instability (Hagmann, M., Science 286:2433-4 (1999), Hartwell, L. H., and M. B. Kastan, Science 266:1821-8 (1994), Nyberg, K. A., et al., Annu Rev Genet 36:617-56 (2002)). hRad9, a member of the Rad family of checkpoint proteins, is involved in detection of DNA damage, cell cycle arrest, and DNA repair (Bessho, T., and A. Sancar., J Biol Chem 275:7451-4 (2000), Greer, D. A., et al., p. 4829-35, Cancer Res, vol. 63 (2003), Lieberman, H. B., et al., Proc Natl Acad Sci USA 93:13890-5 (1996), Weinert, T. A., and L. H. Hartwell, Science 241:317-22 (1988)). The N-terminus of hRad9 shares a region of sequence similarity to the proliferating cell nuclear antigen (PCNA) and associates with Rad1 and Hus1 in a head-to-tail manner, thus forming a stable heterotrimeric DNA sliding clamp (Venciovas, C., and M. P. Thelen, Nucleic Acids Res 28:2481-93 (2000), Volkmer, E., and L. M. Karnitz, J Biol Chem 274:567-70 (1999), Zou, L., et al., Genes Dev 16:198-208 (2002)). Recent studies suggest hRad9 may interact with the anti-apoptotic Bcl-2 family proteins, Bcl-2 and Bcl-xL, through a BH3 domain at its N-terminus (Komatsu, K., et al., Nat Cell Biol 2:1-6 (2000), Yoshida, K., et al., Mol Cell Biol 22:3292-300 (2002)). Therefore, in addition to its previously reported checkpoint-control functions, hRad9 may play a role in regulating apoptosis.

Disclosed herein hRad9 interacts with AR in an androgen-dependent manner. It is shown that the FXXLF motif at the C-terminus of hRad9 mediates the interaction with the AR LBD. The results also show that hRad9 down-regulates AR transcriptional activation through blocking the N/C interaction of AR. This is an embodiment of checkpoint proteins crosstalking with AR signaling in prostate cancers.

(b) Domains of AR Involved in Binding to hRad9

The amino acid 327 to 391 fragment of bRad9 interacted with the AR-DBD-LBD and AR-LBD, but very little with the AR-DBD in the presence of DHT. However, the interaction was stronger with the AR-DBD-LBD than with the LBD alone, and this could indicate the DBD aids in the folding of the LBD in yeast.

(c) FXXLF Motif Mediates AR-hRad9 Interaction

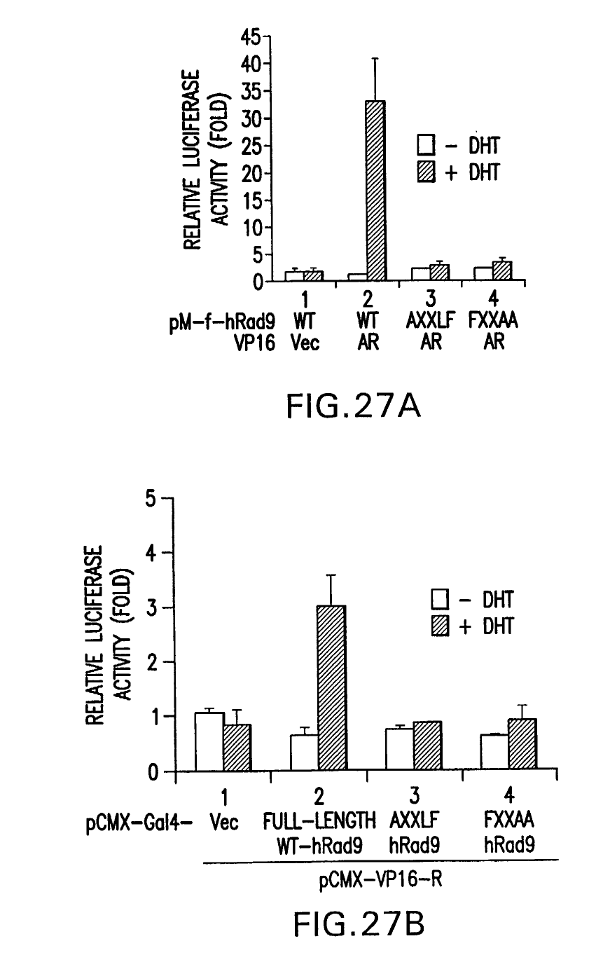

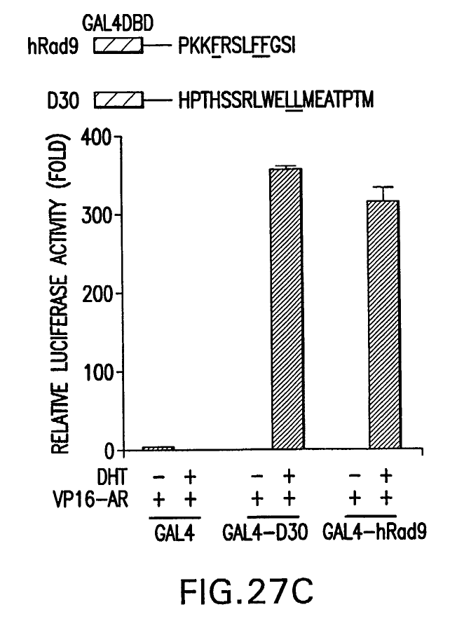

The LXXLL motif was first identified in some SR coactivators (Heery, D. M., et al., Nature 387:733-6 (1997)). However, among steroid receptors, AR appears to be relatively unique as it interacts with only a very limited subset of LXXLL sequences (Chang, C. Y., and D. P. McDonnell., Mol Endocrinol 16:647-60 (2002)). Previous studies showed that the FXXLF motif plays important roles in mediating the interaction of the AR LBD with several FXXLF-containing AR coregulators (He, B., et al., J Biol Chem 275:22986-94 (2000); He, B., et al., J Biol Chem 277:10226-35 (2002)). Interestingly, one FXXLF motif is located at the carboxyl-terminus of hRad9 (aa 361-365). Mutations of the FXXLF motif in Rad9 decreased dramatically the interaction between AR and the fragment of hRad9 (aa 327-391), shown by either the AXXLF or FXXAA mutants (FIG. 27A, lane 3, 4 vs. lane 2, closed bars). Similarly, AXYXLF or FXXAA mutants reduced the interaction between AR and full-length hRad9 (FIG. 27B, lane 3, 4 vs. lane 2, closed bars), indicating this FXXLF motif is critical for hRad9 to interact with AR. Small hRad9 FXXLL peptides (FIG. 27C) (PKKFRSLFFGSI, SEQ ID NO:22) interacted with AR, indicating that the FXXLL in hRad9 with a few amino acids surrounding this sequence was sufficient to mediate the interaction between hRad9 and AR (Another FXXLL peptide, is D30, HPTHSSRLWELLMEATPTM, SEQ ID NO:23).

(d) hRad9 Specifically Represses AR-Mediated Transactivation

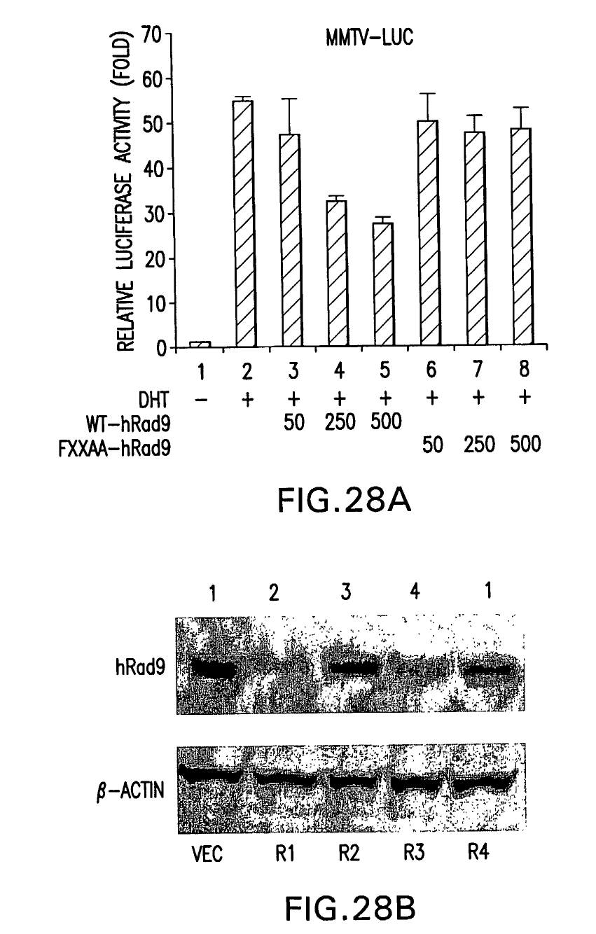

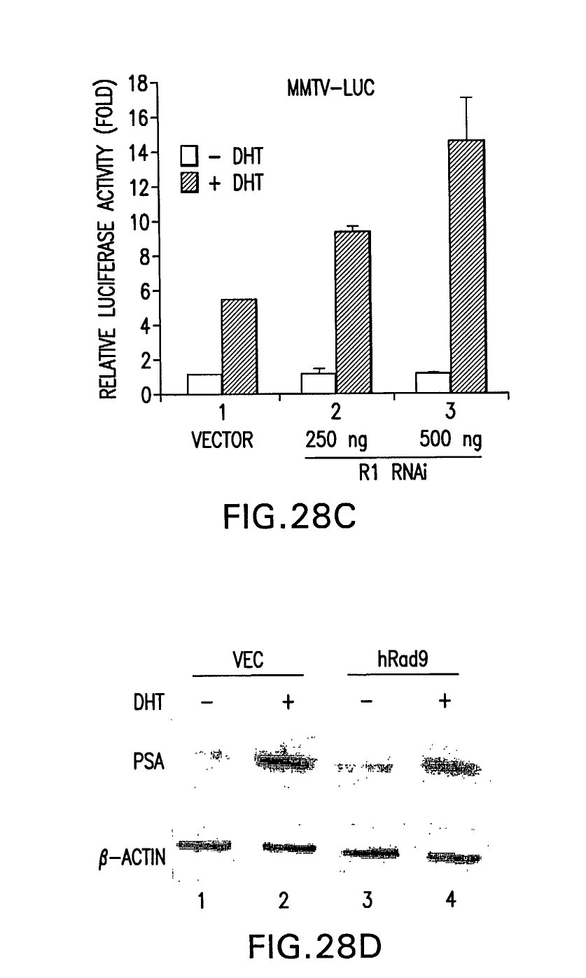

Wild type hRad9 with decreased the transcriptional activity of AR in a dose-dependent manner (FIG. 28A, lanes 3-5), whereas FXXAA mutants had only marginal effect on AR transactivation (FIG. 28A, lanes 6-8). Neither wild type (WT) nor FXXAA mutant of hRad9 had an effect on the transcriptional activity in the absence of 10 nM DHT, indicating that they do not affect the basal transcriptional activity. These results were obtained in both PC-3 and LNCaP cells.

Molecules designed to inhibit hRad9 (siRNA for hRad9) decreased the hRad9 protein levels in both CWR22R cells and PC-3 cells, and in the presence of siRNA for hRad9, transactivation due to AR increased. Furthermore, addition of hRad9 decreased the production of PSA in LNCaP cells after inducement with DHT.

The FXXLF motif in AR N-terminus is important for interacting with the C-terminus of AR and this interaction is required for fall capacity of AR transactivation (Hsu, C. L., et al., J Biol Chem 278:23691-8 (2003)). Disclosed herein one mechanism by which hRad9 can affect the tranactivation activity of AR is through disruption of the AR N/C interaction by the hRad9 FXXLF motif might.

(4) Antibodies

Disclosed are antibodies that bind the ARA67, AR, GSK2B, or hRad9, for example. In certain embodiments, the antibodies bind AR, such that the antibodies mimic the binding of ARA67, GSK2B, or hRad9 to AR. This mimicking can occur through, for example, competitively binding with ARA 67, GSK2B, or hRad9. These antibodies can be isolated by for example, raising antibodies to AR, as disclosed herein, and then assaying the hybridomas for antibodies that are competed off with ARA67, GSK2B, or bRad9, for example. The antibodies can also be identified by assaying their performance in the disclosed AR activity assays herein, and comparing that activity in the presence of the antibody to, for example, the activity in the presence of ARA67, GSK2B, or hRad9, for example.

(a) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as described herein. The antibodies are tested for their desired activity using the in vitro assays described herein, or by analogous methods, after which their in in vivo therapeutic and/or prophylactic activities are tested according to known clinical testing methods.

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V (H)) followed by a number of constant domains. Each light chain has a variable domain at one end (V (L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarily determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a b-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md. (1987)). The constant domains are not involved directly in binding an antibody to an antigen, but exhibit various effector functions, such as participation of the antibody in antibody-dependent cellular toxicity.

As used herein, the term “antibody or fragments thereof” encompasses chimeric antibodies and hybrid antibodies, with dual or multiple antigen or epitope specificities, and fragments, such as scFv, sFv, F (ab′)₂, Fab′, Fab and the like, including hybrid fragments. Thus, fragments of the antibodies that retain the ability to bind their specific antigens are provided. For example, fragments of antibodies which maintain ARA67, AR, GSK2B, or hRad9, for example, binding activity or mimic ARA67, AR, GSK2B, or hRad9, for example, binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods of the invention serves to lessen the chance that an antibody administered to a human will evoke an undesirable immune response.

(b) Human Antibodies

The human antibodies of the invention can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147 (1):86-95, 1991). Human antibodies of the invention (and fragments thereof) can also be produced using phage display libraries (Hoogenboom et al., J. Mol. Biol., 227:381, 1991; Marks et al., j Mol. Biol., 222:581, 1991).

The human antibodies of the invention can also be obtained from transgenic animals. For example, transgenic, mutant mice that are capable of producing a fall repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J (H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

(c) Humanized Antibodies

Optionally, the antibodies are generated in other species and “humanized” for administration in humans. Humanized forms of non-human (e.g., murine) antibodies are chimeric immunoglobulins, immunoglobulin chains or fragments thereof (such as scFv, sFv, Fv, Fab, Fab′, F (ab′)₂, or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. Ea general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody (Sims et al., J. Immunol., 151:2296 (1993) and Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational strictures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen (s), is achieved. In general, the CDR residues are directly and most substantially involved in influencing antigen binding (see, WO 94/04679, published 3 Mar. 1994).

(d) Monoclonal Antibodies

The term monoclonal antibody as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain (s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

Monoclonal antibodies of the invention can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro, e.g., using the complexes described herein.

Transgenic animals (e.g., mice) that are capable, upon immunization, of producing a fall repertoire of human antibodies in the absence of endogenous immunoglobulin production can be employed. For example, it has been described that the homozygous deletion of the antibody heavy chain joining region (J (H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., Proc. Natl. Acad. Sci. USA, 90:2551-255 (1993); Jakobovits et al., Nature, 362:255-258 (1993); Bruggemann et al., Year in Immuno., 7:33 (1993)). Human antibodies can also be produced in phage display libraries (Hoogenboom et al., J. Mol. Biol., 227:381 (1991); Marks et al., J. Mol. Biol., 222:581 (1991)). The techniques of Cote et al. and Boerner et al. are also available for the preparation of human monoclonal antibodies (Cole et al., Monoclonal Antibodies and Cancer Therapy, Alan R. Liss, p. 77 (1985); Boerner et al., J. Immunol., 147 (1):86-95 (1991)).

Generally, either peripheral blood lymphocytes (“PBLs”) are used in methods of producing monoclonal antibodies if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, “Monoclonal Antibodies: Principles and Practice” Academic Press, (1986) pp. 59-103). Immortalized cell lines are usually transformed mammalian cells, including myeloma cells of rodent, bovine, equine, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells. Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J. Immunol., 133:3001 (1984); Brodeur et al., “Monoclonal Antibody Production Techniques and Applications” Marcel Dekker, Inc., New York, (1987) pp. 51-63). The culture medium in which the hybridoma cells are cultured can then be assayed for the presence of monoclonal antibodies directed against ARA67, AR, GSK2B, or hRad9, for example. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art, and are described further in the Examples below or in Harlow and Lane “Antibodies, A Laboratory Manual” Cold Spring Harbor Publications, New York, (1988).

After the desired hybridoma cells are identified, the clones may be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, protein G, hydroxylapatite chromatography, gel electrophoresis, dialysis, or affinity chromatography.

The monoclonal antibodies may also be made by recombinant DNA methods, such as those described in U.S. Pat. No. 4,816,567 (Cabilly et al.). DNA encoding the monoclonal antibodies of the invention can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

(e) Antibody Fragments

Also disclosed are fragments of antibodies which have bioactivity. The polypeptide fragments of the present invention can be recombinant proteins obtained by cloning nucleic acids encoding the polypeptide in an expression system capable of producing the polypeptide fragments thereof, such as an adenovirus or baculovirus expression system. For example, one can determine the active domain of an antibody from a specific hybridoma that can cause a biological effect associated with the interaction of the antibody with ARA67, AR, GSK2B, hRad9, TR2, or TR4, for example. For example, amino acids found to not contribute to either the activity or the binding specificity or affinity of the antibody can be deleted without a loss in the respective activity. For example, in various embodiments, amino or carboxy-terminal amino acids are sequentially removed from either the native or the modified non-immunoglobulin molecule or the immunoglobulin molecule and the respective activity assayed in one of many available assays. In another example, a fragment of an antibody comprises a modified antibody wherein at least one amino acid has been substituted for the naturally occurring amino acid at a specific position, and a portion of either amino terminal or carboxy terminal amino acids, or even an internal region of the antibody, has been replaced with a polypeptide fragment or other moiety, such as biotin, which can facilitate in the purification of the modified antibody. For example, a modified antibody can be fused to a maltose binding protein, through either peptide chemistry or cloning the respective nucleic acids encoding the two polypeptide fragments into an expression vector such that the expression of the coding region results in a hybrid polypeptide. The hybrid polypeptide can be affinity purified by passing it over an amylose affinity column, and the modified antibody receptor can then be separated from the maltose binding region by cleaving the hybrid polypeptide with the specific protease factor Xa. (See, for example, New England Biolabs Product Catalog, 1996, pg. 164). Similar purification procedures are available for isolating hybrid proteins from eukaryotic cells as well.

The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment. These modifications can provide for some additional property, such as to remove or add amino acids capable of disulfide bonding, to increase its bio-longevity, to alter its secretory characteristics, etc. In any case, the fragment must possess a bioactive property, such as binding activity, regulation of binding at the binding domain, etc. Functional or active regions of the antibody may be identified by mutagenesis of a specific region of the protein, followed by expression and testing of the expressed polypeptide. Such methods are readily apparent to a skilled practitioner in the art and can include site-specific mutagenesis of the nucleic acid encoding the antigen. (Zoller M J et al. Nucl. Acids Res. 10:6487-500 (1982).

A variety of immunoassay formats may be used to select antibodies that selectively bind with a particular protein, variant, or fragment. For example, solid-phase ELISA immunooassays are routinely used to select antibodies selectively immunoreactive with a protein, protein variant, or fragment thereof. See Harlow and Lane. Antibodies, A Laboratory Manual. Cold Spring Harbor Publications, New York, (1988), for a description of immunoassay formats and conditions that could be used to determine selective binding. The binding affinity of a monoclonal antibody can, for example, be determined by the Scatchard analysis of Munson et al., Anal. Biochem., 107:220 (1980).

(f) Administration of Antibodies

Antibodies of the invention are preferably administered to a subject in a pharmaceutically acceptable carrier. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of antibody being administered.

The antibodies can be administered to the subject, patient, or cell by injection (e.g., intravenous, intraperitoneal, subcutaneous, intramuscular), or by other methods such as infusion that ensure its delivery to the bloodstream in an effective form. Local or intravenous injection is preferred.

Effective dosages and schedules for administering the antibodies may be determined empirically, and making such determinations is within the skill in the art. Those skilled in the art will understand that the dosage of antibodies that must be administered will vary depending on, for example, the subject that will receive the antibody, the route of administration, the particular type of antibody used and other drugs being administered. Guidance in selecting appropriate doses for antibodies is found in the literature on therapeutic uses of antibodies, e.g., Handbook of Monoclonal Antibodies, Ferrone et al., eds., Noges Publications, Park Ridge, N. J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

(g) Nucleic Acid Approaches for Antibody Delivery

The ARA67, AR, GSK2B, hRad9, TR2, or TR4, for example, antibodies and antibody fragments of the invention can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment.

d) Compositions Identified by Screening with Disclosed Compositions/Combinatorial Chemistry

(1) Combinatorial Chemistry

The disclosed compositions can be used as targets for any combinatorial technique to identify molecules or macromolecular molecules that interact with the disclosed compositions in a desired way. The nucleic acids, peptides, and related molecules disclosed herein can be used as targets for the combinatorial approaches. Also disclosed are the compositions that are identified through combinatorial techniques or screening techniques in which the compositions have the sequences disclosed herein, or portions thereof, are used as the target in a combinatorial or screening protocol.

It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as, ARA67, AR, GSKB2, or hRad9, for example, are also disclosed. Thus, the products produced using the combinatorial or screening approaches that involve the disclosed compositions, such as, ARA67, AR, GSKB2, or hRad9, for example, are also considered herein disclosed.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry)

A preferred method for isolating proteins that have a given function is described by Roberts and Szostak (Roberts R. W. and Szostak J. W. Proc. Natl. Acad. Sci. USA, 94 (23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94 (23)12997-302 (1997)).

Another preferred method for combinatorial methods designed to isolate peptides is described in Cohen et al. (Cohen B. A., et al., Proc. Natl. Acad. Sci. USA 95 (24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain. A peptide of choice, for example a portion of ARA67, AR, GSKB2, or hRad9, for example, is attached to a DNA binding domain of a transcriptional activation protein, such as Gal 4. By performing the Two-hybrid technique on this type of system, molecules that bind the desired portion of ARA67, AR, GSKB2, or hRad9, for example, can be identified.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

Screening molecules similar to ARA67, GSKB2, or hRad9, for example, for inhibition of binding to AR, for example, is a method of isolating desired compounds.

Molecules isolated which bind AR, for example, can either be competitive inhibitors or non-competitive inhibitors of the interaction between AR and ARA67, GSKB2, or hRad9, for example. In certain embodiments the compositions are competitive inhibitors of the interaction between AR and ARA67, GSKB2, or hRad9, for example.

In another embodiment the inhibitors are non-competitive inhibitors of the interaction between AR and ARA67, GSKB2, or hRad9, for example. One type of non-competitive inhibitor will cause allosteric rearrangements which mimic the effect of the interaction between Ar and of the interaction between AR and ARA67, GSKB2, or hRad9, for example.

As used herein combinatorial methods and libraries included traditional screening methods and libraries as well as methods and libraries used in interative processes.

(2) Computer Assisted Drug Design

The disclosed compositions can be used as targets for any molecular modeling technique to identify either the structure of the disclosed compositions or to identify potential or actual molecules, such as small molecules, which interact in a desired way with the disclosed compositions. The nucleic acids, peptides, and related molecules disclosed herein can be used as targets in any molecular modeling program or approach.

It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, such as, AR, ARA67, GSKB2, or hRad9, for example, are also disclosed. Thus, the products produced using the molecular modeling approaches that involve the disclosed compositions, such as, of the interaction between AR, ARA67, GSKB2, or hRad9, for example, are also considered herein disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

### C. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular AR is disclosed and discussed and a number of modifications that can be made to a number of molecules including the AR are discussed, specifically contemplated is each and every combination and permutation of AR and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

1. Homology/Identity

It is understood that one way to define any known variants and derivatives or those that might arise, of the disclosed genes and proteins herein is through defining the variants and derivatives in terms of homology to specific known sequences. For example SEQ ID NO:2 sets forth a particular sequence of an ARA67 and SEQ ID NO:1 sets forth a particular sequence of the protein encoded by SEQ ID NO:2, an ARA67 protein. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

2. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarily desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

a) Sequences

There are a variety of sequences related to the ARA67, AR, GSK2B, hRad9, TR2, or TR4, for example, and other disclosed genes having the following Genbank Accession Numbers: (SEQ ID NO:1) ARA67 protein, AAH18121; (SEQ ID NO:2) ARA67 DNA, BC018121; (SEQ ID NO:3), AR protein and DNA, NM_{—}000044; (SEQ ID NO:5), GSK3B protein, NP_{—}002084); SEQ ID NO:6 GSK3B DNA, NM_{—}002093); SEQ ID NO:7 hRAD9 protein, AAB39928; SEQ ID NO:8 BRAD 9 cDNA, U53174; SEQ ID NO:13 TR2 protein, M21985; SEQ ID NO:14 TR4 protein, P49116; SEQ ID NO:15 TR2 cDNA, Accession No. M21985; SEQ ID NO:16 TR4 cDNA, P49116, these sequences and others are herein incorporated by reference in their entireties as well as for individual subsequences contained therein.

One particular sequence set forth in SEQ ID NO:3 and having Genbank accession number NM_{—}000044 is used herein, as an example, to exemplify the disclosed compositions and methods. It is understood that the description related to this sequence is applicable to any sequence disclosed herein unless specifically indicated otherwise. Those of skill in the art understand how to resolve sequence discrepancies and differences and to adjust the compositions and methods relating to a particular sequence to other related sequences (i.e. sequences of AR). Primers and/or probes can be designed for any AR sequence given the information disclosed herein and known in the art.

3. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991) Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as ARA67, AR, GSK2B, hRad9, TR2, or TR4, for example, into the cell without degradation and include a promoter yielding expression of the gene in the cells into which it is delivered. In some embodiments the ARA67, AR, GSK2B, hRad9, TR2, or TR4, for example, are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

(3) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof; confer infectivity and site-specific integration, but not cytotoxicity, and the promoter directs cell-specific expression. U.S. Pat. No. 6,261,834 is herein incorporated by reference for material related to the AAV vector.

The vectors of the present invention thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(4) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed ARA67, AR, GSK2B, hRad9, TR2, or TR4, for example, or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci. USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

c) In Vivo/Ex Vivo

As described above, the compositions can be administered in a pharmaceutically acceptable carrier and can be delivered to the subject=s cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

4. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now liown from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

5. Peptides

a) Protein Variants

As discussed herein there are numerous variants of the ARA67, AR, GSK2B, hRad9, TR2, or TR4, for example, proteins that are known and herein contemplated. In addition, to the known functional ARA67, AR, GSK2B, hRad9, TR2, or TR4, for example, strain variants there are derivatives of the ARA67, AR, GSK2B, hRad9, TR2, or TR4, for example, proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for malting substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions. 240.

TABLE 1
Amino Acid Abbreviations

| Amino Acid | Abbreviations |
|---|---|
| alanine | Ala A |
| allosoleucine | AIle |
| arginine | Arg R |
| asparagine | Asn N |
| aspartic acid | Asp D |
| cysteine | Cys C |
| glutamic acid | Glu E |
| glutamine | Gln Q |
| glycine | Gly G |
| histidine | His H |
| isolelucine | Ile I |
| leucine | Leu L |
| lysine | Lys K |
| phenylalanine | Phe F |
| proline | Pro P |
| pyroglutamic | pGlu |
| acidp | |
| serine | Ser S |
| threonine | Thr T |
| tyrosine | Tyr Y |
| tryptophan | Trp W |
| valine | Val V |

TABLE 2
Amino Acid Substitutions
Original Residue Exemplary Conservative Substitutions,
others are known in the art.
Ala; Ser
Arg; Lys; Gln
Asn; Gln; His
Asp; Glu
Cys; Ser
Gln, Asn, Lys
Glu; Asp
Gly; Pro
His; Asn; Gln
Ile; Leu; Val
Leu; Ile; Val
Lys; Arg; Gln;
Met; Leu; Ile
Phe; Met; Leu; Tyr
Ser; Thr
Thr; Ser
Trp; Tyr
Tyr; Trp; Phe
Val; Ile; Leu

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulls side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trains), —COCH₂—, —CH(OH)CH₂—, and —CHH₂SO—(These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. 1307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

It is understood that one way to define the variants and derivatives of the disclosed proteins herein is through defining the variants and derivatives in terms of homology/identity to specific known sequences. For example, SEQ ID NOs:1, 3, 5, 7, 13, and 14 set forth a particular sequence of ARA67, AR, GSK2B, hRad9, TR2, or TR4 proteins, respectively. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences, such as ARA67, AR, GSK2B, hRad9, TR2, or TR4, for example, it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein in the particular organism from which that protein arises is also known and herein disclosed and described.

6. Antiandrogens and Molecules Modulating Hormonal Secretion

There are a number of different types of molecules functioning as antiandrogens and molecules modulating hormonal secretion that can be used in androgen receptor/androgen related cancer therapies, such as prostate cancer therapies. For example, hormonal secretion from the hypothalamus can be modulated by LH-RH agonists, such as Lupron (Formula 3, Cas Nr 0053714-56-0) 5′oxo-Pro-His-Trp-Ser-Tyr-Dleu-Leu-Arg-Pro-NH—CH₂—CH₃ and Zoladex, (Formula 4, Cas Nr. 0065807-02-5)

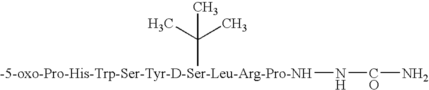

which inhibit the production of Testosterone (T) by the testes and adrenal glands. There are also anti-androgen therapeutics, such as Flutamide (Formula 5, 0013311-84-7)

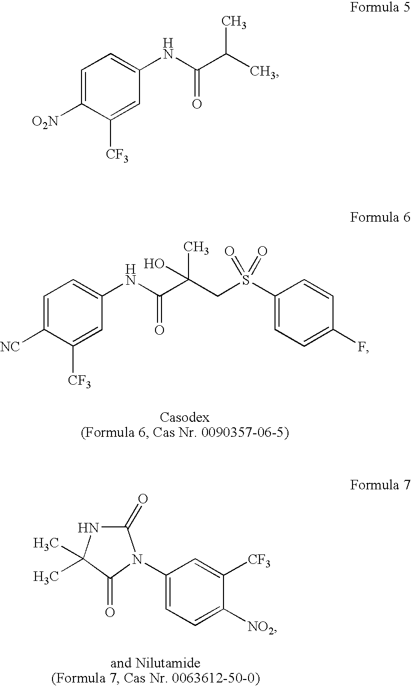

, which can block the androgen binding to AR. Other therapies include the administration of 5-α reductase inhibitors, such as Proscar (Finasteride) (Formula 8 as Nr. 0098319-26-7)

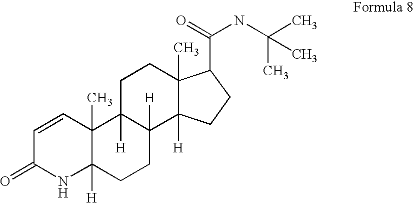

, which can inhibit the conversion of T to DHT. DHT is the most effective ligand for AR with higher binding affinity that T. However, this compound is generally applied for BPH patients rather than for prostate cancer patients.

Estrogen, such as DES, estradiol, and Stilphosterol Honvan, have also been used in the treatment of prostate cancer. These molecules can decrease the amount of hormones from the hypothalamus. These molecules can decrease the T synthesis from testis by inducing a negative feed-back regulation in leutinizing hormone (LH) secretion from the pituitary gland and gonadotropin releasing hormone (GnRH) secretion from the hypothalamus. Other therapeutics include Ketoconazole (Nizoral), which can inhibit the cytochrome p459 enzyme system to reduce T synthesis, and steroids such as Hydrocortisone, Aminoglutethemide (Cytadren), dexmethasome (Decadron), and Cyproterone (Androcur). Ketoconazole is usually used as a second line hormone therapy in patients with stage IV recurrent prostatic cancer. Aminoglutethimide (Cytadren) blocks adrenal steroidogenesis by inhibiting the enzymatic conversion of cholesterol to pregnenolone. Cypoterone is a steroidal antiandrogen with weak progestational activity that results in the partial suppression of pituitary gonadotropin and a decrease in serum T. The main purpose of using Hydrocortisone and Decadron is to relieve the symptoms and increase the quality of life of prostate cancer patients. It is understood that combinations of these therapeutics are performed and herein disclosed.

Thus, disclosed are anti-prostate cancer compounds, such as, flutamide/HF, casodex, niflutamide, finasteride, 1,25-dihydroxyl, vitamin D3, and natural products including quercetin, resveratrol, silymarin, isoflavonoids, epigallocatechin gallate (EGCG), docosahexaenoic acid (DHA) and eicosapentaenoic acid (EPA). These and others, can all be added in combination with the molecules disclosed herein that inhibit androgen independent activity of AR, such as ARA67, GSK2B, and hRad9, and various fragments. These can be used collectively or individually in any combination.

Typically, the antiandrogens and antihormone cancer compounds can be provided at concentrations of less than or equal to 20 uM, 15 uM, 10, uM, 5 uM, 2 uM, 1 uM, 0.1 uM, or 0.01 uM. Typically the other disclosed inhibitors, can be administered at concentrations of less than or equal to 1 mM, 0.5 mM, 100 uM, 90 uM, 80 uM, 70 uM, 60 uM, 50 uM, 40 uM, 30 uM, 20 uM, 15 uM, 10, uM, 5 uM, 2 uM, 1 uM, 0.1 uM, or 0.01 uM. Furthermore, typically, anticancer agents will be dosed at a 0.1-10 mg/kg range and at times they can fall into a 0.01-30 mg/kg range depending on the bioactivity of the compounds. Furthermore administration depends on patient body weight and disease state and can be determined. Those of skill in the art understand how to assay for the optimal concentration for administration in vivo, of any of the disclosed compositions, by for example, relying on disclosed cell and animal models for action, as well as by testing the compositions in vivo at various concentrations.

7. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, although topical intranasal administration or administration by inhalant is typically preferred. As used herein, “topical intranasal administration” means delivery of the compositions into the nose and nasal passages through one or both of the nares and can comprise delivery by a spraying mechanism or droplet mechanism, or through aerosolization of the nucleic acid or vector. The latter may be effective when a large number of animals is to be treated simultaneously. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution or suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

8. Compositions with Similar Functions

It is understood that the compositions disclosed herein have certain functions, such as binding AR or inhibiting AR function, such as non-androgen related AR activity. Disclosed herein are certain structural requirements for performing the disclosed functions, and it is understood that there are a variety of structures which can perform the same function which are related to the disclosed structures, and that these structures will ultimately achieve the same result, for example, inhibition of non-androgen related AR activity.

### D. METHODS OF MAKING THE COMPOSITIONS

The compositions disclosed herein and the compositions necessary to perform the disclosed methods can be made using any method known to those of skill in the art for that particular reagent or compound unless otherwise specifically noted.

Disclosed are animals produced by the process of transecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

Also disclose are animals produced by the process of adding to the animal any of the cells disclosed herein.

### E. METHODS OF USING THE COMPOSITIONS

1. Method of Treating Cancer

The disclosed compositions can be used to treat any disease where uncontrolled cellular proliferation occurs such as cancers and which are related to AR. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general. A representative but non-limiting list of cancers that the disclosed compositions can be used to treat is the following: bladder cancer, kidney cancer, prostate cancer, colon cancer, breast cancer, renal cancer, genitourinary cancer, large bowel cancer, and testicular cancer.

### F. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### a) Materials and Methods

(1) Generation of Female AR−/− Mice

Construction of targeting vectors and generation of the chimera founder mice have been described previously (Yeh et al. 2002). The strain of the mosaic founder was C57BL/6-129/SEVE. β-Actin is a housekeeping gene and is universally expressed in every tissue. Therefore, the β-actin promoter driven Cre (ACTB-Cre) will express and delete the floxed AR fragment in all the cells. The mating strategy is briefly illustrated in FIG. 1. The female AR^{-/-} mice were genotyped by PCR, rather than by Southern blot analysis, as described in figure legend and previously (Yeh et al. 2002). (Also see PCT application, PCT/US02/24234, which discloses androgen receptor knockouts and is herein disclosed and incorporated by reference at least for material related to androgen receptor knockouts)

(2) Animal, Tissue Collection, and Real-Time RT-PCR.

All animal experimentation was conducted in accordance with accepted standards of humane animal care. Tissues were fixed in 4% paraformaldehyde for 24 h at 4° C. Paraffin-embedded tissues were sectioned (5-7 μm) onto Probe-On Plus charged slides (Fisher Scientific, Pittsburgh, Pa.). Fresh mammary gland tissues were frozen in liquid nitrogen and stored at −80° C. before RNA extraction. 3 μg of total RNA was reverse-transcribed (RT) and subjected to real-time PCR using iCycle (Bio-Rad). Primer sequences were designed by Beacon Designer II software (Bio-Rad) and the formula used were described previously (Bieche et al. 2001). β-Actin was used as an internal control.

(3) Whole-Mount Staining of Mammary Glands

Whole mammary glands were fixed overnight in Carnoy's solution (Ethanol:CHCl3:acetic acid, 6:3:1), spread on glass slides, sequentially rehydrated by 100%, 95%, 70%, and 50% ethanol and tap water, then stained in carmine red solution overnight till the whole gland become red. After staining, the tissue slides were dehydrated, cleared with xylene, and mounted.

(4) Immunohistochemistry (IHC) and BrdUstaining

Mammary glands were fixed overnight in buffered neutral formalin (VWR Scientific Products) at room temperature. The tissues were dehydrated by passing through 70%, 85%, 95%, and 100% ethanol, cleared in xylene, then 1:1 xylene:paraffin for 45 min, and embedded in paraffin. Tissue sections were cut at 4-elm and mounted onto slides.

For IHC, sections were heated at 55° C. at least 2 h, deparaffinized in xylene, rehydrated, and washed in Tris-buffered saline (TBS)/pH 8.0. For antigen retrieval, slides were microwaved in 0.01 M sodium citrate/pH 6.0, and immersed with 1% hydrogen peroxide in methanol for 30 min, then blocked with 20% normal goat serum in TBS for 60 min. After washing with PBS, sections were incubated for 90 min in different antibody of diluted 1:200 to 1:500 in TBS containing 1% bovine serum albumin (BSA), followed by goat anti-rabbit biotinylated secondary antibody diluted 1:300 in TBS containing 1% BSA. Then sections were incubated with ABC solution for 30 min, followed by development with DAB peroxidase substrate kit (Vector Laboratories) for 5 min. Slides were counterstained with hematoxylin for 30 sec, then dehydrated, cleaned in xylene, and mounted. Primary antibody was replaced with normal rabbit IgG or 1% BSA in TBS for negative controls. For BrdU labeling, BrdU reagent and BrdU staining kit were purchased from Zymed Laboratories.

(5) Steroid Hormone RIA

For characterization of hormonal profiles, blood samples obtained from AR^{+/+} and AR^{-/-} mice by the intracardiac method under ketamine and xylazine anesthesia (Sigma) were collected into centrifuge tubes containing 50 mM EDTA. Estrogen and progesterone were checked using Coat-a-Count kits (Diagnostic Products).

(6) Statistical Analysis

All data were analyzed by one-way ANOVA using minitab statistical software (State College, Pa.). Mean separation was accomplished using Fisher's pairwise comparison. Differences were considered significant at p<0.05.

(7) Construction of the AR Targeting Vector and Generation of AR−/− MCF7 Cells

The targeting vector for generating AR^{-/-} MCF7 cells was constructed by replacing the SmaI-KpnI segments within the AR exon 1 with a promoterless neomycin cassette and inserting two flanking sequences, 5′ extending 1.1 kb into the human AR 5′ UTR and 3′ extending 6.2 kb into the AR intron 1, on a pGEM-T easy vector (Promega). This promoterless neomycin cassette was inserted in frame with AR ATG and contains a termination codon and a polyadenylation signal. The flanking homologous sequences were generated by PCR using the genomic DNA from human LNCaP cells as template. For generation of AR^{-/-} MCF7 cells, parental MCF7 cells were transfected with the AatII-linearized AR targeting vector using SuperFect (Qiagen) and then selected with neomycin (400 μg/ml). The genotypes of surviving clones were screened by Southern blot analyses. The heterozygous clones (AR^{+/+}) were picked up and subjected to the second gene targeting experiment using the same targeting vector. Clones were then selected with a higher concentration of neomycin (1.25 mg/ml). The genotypes of surviving clones were again screened by Southern blot analyses.

(8) Construction of AR siRNA Expression Plasmid

A small interfering RNA was expressed in mammalian cells by transfection of a DNA-based vector BS/U6 (Sui et al. 2002) containing a homologous sequence (GGGCCCCTGGATGGA-TAGCTAC SEQ ID NO:9), a 6-bp spacer (CTCGAG), an inverted homologous sequence (GTAGCTATCCATCCAGGGGCC SEQ ID NO:10), and 5 Ts, at the transcription initiation site of the U6 promoter. See SEQ ID NO. 11 and 12 for fall AR siRNA.

(9) MTT Growth Assays

104 cells seeded on 24-well plates were cultured with RPMI 1640 supplemented with 10% of CDS-FBS (charcoal-dextran-stripped BBS) for treatment with 0.1 nM E2 or 0.2% of HI-FBS (heat-inactivated FBS) for treatment with 100 ng/ml IGF-1 or 50 ng/ml EGF. The cells were collected at indicated days for MTT assay according to the manufacturer's instructions (Sigma).

(10) Soft-Agar Colony Formation Assay

2×104 cells suspended in 0.4% low melting agarose (FMC) were layered on top of 1 ml of 0.8% agarose in 6-well culture plates. Cells were incubated with 1 ml RPMI 1640 supplemented with 10% CDS-FBS for treatment with 0.1 nM E2 or 0.2% HI-FBS for treatment with 100 ng/ml HRG-α After 4 weeks of incubation, the colonies were visualized by staining with 1 mg/ml INT (Sigma) for 24 h and counted with VersaDoc Imaging System (Bio-Rad).

(11) Reporter Gene Assays

Cells were plated in 96-well plates and plasmids at 0.5 μg per well were transfected into cells using SuperFect (Qiagen). The medium was changed 2 h after transfection and cells were cultured in the medium containing 10% CDS-FBS or 0.2% HI-FBS for 16 h, followed by treatment with 50 ng/ml EGF, 100 ng/ml IGF-1,100 ng/ml HRG-α 0.1 nM E2, or 1 nM DHT for another 16 h. Cells were then harvested and the luciferase activity was analyzed using Dual-Luciferase Reporter Assay System (Promega). 5 ng pRL-TK per well was used as internal control.

(12) Western Blots

Cells were lysed with RIPA buffer containing 0.5% Nonidet P-40 and Western blotted with anti-AR (NH-27), anti-ER, anti-actin (Santa Cruz), anti-MAPK, and anti-phosphoMAPK (Cell Signaling).

b) Results

Generation and phenotype of female AR−/− mice was performed using a Cre-lox conditional knockout strategy by mating the floxed AR male mice with AR^{+/-} ACTB Cre⁺ females, and thus, female AR^{-/-} mice and AR^{-/-} ACTB Cre⁺ (FIGS. 1A, B) were generated (See PCT application, PCT/US02/24234, which discloses androgen receptor knockouts and is herein disclosed and incorporated by reference at least for material related to androgen receptor knockouts). Adult female mice with homologous deletion of AR appear healthy and develop normal external genitalia. Gross anatomical examination did not reveal obvious differences in the morphology of most organs between the AR^{+/+} and AR^{-/-} littermates. The body weights were similar between the AR^{+/+} and AR−/− mice, but the thymus of female AR^{-/-} mice was bigger than that of female AR^{+/+} or AR^{+/-} mice. Several estrogen target organs, including mammary gland, ovary, oviduct, and uterus, were collected from 4-, 6-, and 12-wk-old mice. The weight of these organs was 15-23% less in female AR^{-/-} mice as comparing to their age-matched littermates.

(1) Defects of Mammary Gland Development in Prepubertal and Pubertal Stages in Female AR^{-/-} Mice

The morphology of mammary glands was compared between immature (4-wk-old and 6-wk-old) virgin AR^{+/+} and AR^{-/-} mice. At the 4th-6th week, the ductal system had about 50% less extension in female AR^{-/-} mice with reduced numbers and size of the terminal end buds (TEBs) (FIGS. 1C,E). Bromodeoxyuridine (BrdU) staining also revealed a 50% lower proliferation of AR−/− mammary glands, as compared with that of ART^{+/+} mice (FIGS. 1D,E). The size and number of Cap cells (Silberstein 2001), which are responsible for the ductal extension from TEB, were also reduced in female AR^{-/-} mice (FIG. 1F). Together, the results indicated that the mammary gland development is retarded in prepubertal and pubertal stages in female AR^{-/-} mice.

(2) Reduced Ductal Morphogenesis in the Mammary Glands of the Mature AR^{-/-} Mice

At maturity (8-, 16-, and 20-wk-old), AR^{-/-} mammary glands were filled with large bloated ducts terminating with bloated ends. Also AR^{-/-} mammary glands have fewer secondary and tertiary ductal branches as compared to age-matched AR^{+/-} and AR^{+/+} mice (FIG. 2A-C). During the pregnancy stage, the retarded ductal branches in AR^{-/-} mice are partially restored, yet compared to wt mice, the AR^{-/-} mice mammary glands still have less milk-producing alveoli (FIG. 2D). In agreement with these findings, shrunken ductal spaces were observed in some AR^{-/-} mice mammary glands at 16-wk-old or older mice (FIG. 2E) and abnormal nursing behavior in AR^{-/-} mother. The decreased milk producing alveoli and shrunken ductal spaces resulted in the lessened capacity for AR^{-/-} mice to feed their offspring. In 20-wk-old mice, the mammary glands in AR−/− mice underwent degeneration earlier than those of the AR^{+/+} mice (FIG. 2C). Together, FIG. 2 demonstrates that the lack of AR in female mice can retard the mammary gland development and affect the capacity of female AR^{-/-} mice to feed their offspring.

(3) Defects of MAPK Activity and IGF-I/IGF-IR Pathway in AR^{-/-} Mammary Glands

Early studies indicated that major factors, including E2/ER, P/PR, and paracrine growth factors/MAPK signals, may contribute to the growth and development of mammary glands (Lydon et al. 1995; Niemann et al. 1998; Couse and Korach 1999). Immunostaining data show that the phospho-MAPK expression is weaker in the mammary cells from AR^{-/-} mice, compared with AR^{+/+} mice (FIGS. 3A, B, the representative results from 6-wk-old mice are shown), although the total MAPK protein expression is similar between AR^{-/-} and AR^{+/+} mice. Upstream regulators of MAPK signals, IGF-I/IGF-I receptor (IGF-IR) were examined next. It was found that IGF-IR, but not IGF-I, mRNA expression was reduced by 46% in immature female AR^{-/-} mice (FIG. 3C), indicating that the IGF-I/IGF-IR→MAPK signaling pathway may be defective in female AR^{-/-} mice. The expression of the downstream target, cyclin D1, between AR^{-/-} and AR^{+/+} mice was investigated. The cyclin D1 mRNA expression was significantly reduced in female AR^{-/-} mice (FIG. 3C). Similar reduction of the cyclin D1 protein levels, using immunostaining, also occurred. Together, the data showing that the reduction of IGF-IR, cyclin D1, and MAPK activity indicates that the defects in the AR→IGF-I/IGF-IR→MAPK→cyclin D1 signaling pathway can result in the retarded mammary gland development in female AR^{-/-} mice. This is in agreement with early reports showing that IGF-I/IGF-IR is an important paracrine growth factor for mammary gland development (Kleinberg et al. 2000; Bonnette and Hadsell 2001), and cyclin D1 is a downstream mediator of growth factor-induced mammary gland proliferation (Brisken et al. 2002).

Before systematic hormone function which occurs after puberty, growth factors, such as IGF-I, are the major contributing factors to influence the mammary gland development. IGF-I is a potent mitogen for mammary epithelial cells, and the ductual development can be stimulated by IGF-I. The mRNA for IGF-I and IGF-IR are expressed in mammary stroma and developing TEB, and targeted deletion of IGF-IR inhibits normal TEB development before puberty (Bonnette and Hadsell 2001). Disclosed herein, the results indicated that knockout of AR affects the mammary gland development before the puberty stage (FIGS. 1 and 3), which indicated a possible disturbance on the growth factor pathway. Indeed, the results indicated that the reducing of IGF-IR expression consequently affected the IGF-I/IGF-IR signal on development of the mammary gland, including retarded ductual development, less Cap cell in the terminal end bud, and reduced BrdU staining and cyclin D1 expression (FIGS. 1 and 3). The above observations in the prepubertal ARKO mammary glands indicated a tight association between AR and growth factor signals in the mammary gland. Together, the results provided in vivo evidence that AR plays a significant role in the prepubertal mammary gland development.

(4) Reduced ER Activity in AR−/− Mammary Glands

Early studies indicated that MAPK could also influence ER function (Kato et al. 1995), and the cyclin D1 could also be a downstream target gene for ER (Said et al. 1997). Defects in MAPK and cyclin D1 may suggest that ER signals could also be impaired in the female AR^{-/-} mice. Therefore, 5-wk-old mice were ovariectomized, treated with E2 for 2 days, and harvested the mammary glands of the mice for the comparison of the ER activity by examining ER target gene expression between AR^{-/-} and AR^{+/+} mice. It was found that estrogen induced estrogen-responsive finger protein (Efp) (Inoue et al. 1993) and hepatocyte growth factor (HGF) (Jiang et al. 1997) were down-regulated in female AR^{-/-} mice as compared to AR^{+/+} mice (FIG. 3D). Early studies also indicated that both Efp and HGF were important factors for breast cell growth (Niemann et al. 1998; Urano et al. 2002). Interestingly, PR expression in mammary glands was similar between AR^{-/-} and AR^{+/+}. This finding is consistent with a previous report showing PR expression is E2/ER-independent in 5-wk-old or younger mice (Haslam 1988). Nevertheless, the serum levels of PR's ligand, progesterone (P), was reduced in 12- to 16-wk-old adult female AR^{-/-} mice (FIG. 3E), which can result in the reduction of P/PR activity in mature mice. As the P/PR signal pathway plays important roles for the tertiary ductal branching and alveolar development (Lydon et al. 1995), the lower P/PR activity in AR^{-/-} mice can contribute to the retarded branching and lobuloalveolar formation in the development of mature stage mammary glands.

(5) The AR−/− MCF7 Cells Exhibit Severe Defects in Growth and Colony Formation

To further dissect the mechanisms of AR roles in breast at molecular and cellular levels, homologous recombination was applied by using a targeting vector carrying a promoterless neomycin cassette to generate AR-deficient (AR^{-/-}) MCF7 cells (FIG. 4A). Two AR^{-/-} MCF7 clones have been successfully obtained, and the targeted loci were confirmed by Southern blot analysis (FIG. 4B). In these two homologous clones, the expression and the ligand-activated transcriptional activity of AR were indeed abrogated (FIGS. 4C, D). It was found that AR^{-/-} MCF7 cells exhibit a severe impairment in proliferation when cultured in media containing normal, steroid-deprived, or 10⁻¹⁰M E2-treated serum (FIG. 4E). The soft-agar colony formation assay also showed that the colony number of AR^{+/+} MCF7 cells was increased in response to E2 (10⁻¹⁰M) or heregulin-α (HRG-α,100 ng/ml), an activator for the HER2/HER3/HER4 family, whereas the colony formation of AR^{-/-} MCF7 cells was defective (FIG. 4F). A small interfering no RNA (siRNA) was applied to interrupt AR expression in AR^{+/+} MCF7 cells. It was found that AR siRNA-transfected cells had a lower degree of Ki67 immunostaining, and the mRNA levels of Ki67 and c-myc were reduced by 42% and 81%, respectively. Together, FIG. 4 indicates that AR plays an essential role for the growth of breast cancers.

(6) The Growth Factor-Mediated Proliferation and MAPK Activation is impaired in AR^{-/-} MCF7

Next, whether the loss of AR impairs the growth factor-mediated proliferation and MAPK activation in AR^{-/-} MCF7 cells was examined. Treatment of AR^{+/+} MCF7 cells with IGF-I, epidermal growth factor (EGF), or HRG-A could stimulate cell proliferation and activate GAL4-Elk1, a direct target of MAPK, in a low serum-containing medium (FIGS. 5A,B). In contrast, these growth factors-stimulated cell proliferation and MAPK activation was largely impaired in the AR^{-/-}/MCF7 cells (FIGS. 5A, B). Using another strategy by transfection of AR siRNA into AR^{+/+}MCF7 cells, it was found that suppression of AR expression could also diminish IGF-I/EGF/HRG-α-induced MAPK activation (FIG. 5B) and the number of cells entering S-phase of the cell cycle. Moreover, the reduced transcriptional activity of GAL4-Elk1 in AR^{-/-} MCF7 cells could be rescued by transfection of a natural AR promoter (−3.6 k˜+1)-driven AR expression plasmid (np-AR), which contains a full-length AR cDNA flanked with its natural 5′ and 3′ UTRs (FIG. 5C). Interestingly, adding EGF with np-AR can enhance synergistically the transactivation of GAL4-Elk1 in AR−/− MCF7 cells, compared with the cells treated with EGF alone (FIG. 5C), indicating a significant involvement of AR in the growth factor signaling pathway. Furthermore, the AR-activated GAL4-Elk1 activity can be diminished by MAPK phosphatase-1 (CL-100) or a specific inhibitor U0126, as well as dominant-negative Ras or Raf (Sugimoto et al. 1998) (FIG. 5D). Also, the reduction of MAPK activation by AR siRNA can be recovered by constitutively activated MEK (MEK-CA), Ras (Ras-CA), or Raf (Raf-CA), but not by Rac (Sells et al. 1997) (Rac-CA) or PI3K (p110 subunit). These results indicate that AR is an important upstream regulator of the Ras/Raf/MAPK cascade.

(7) The Transcriptional Activity of ER is Defective in AR^{-/-} MCF7 Cells

The ER activity in AR^{-/-} MCF7 cells was examined. The transcriptional activities of ER were reduced by 58.8%, 53.8%, and 55.0% in AR^{-/-} MCF7 cells in the presence of E2 at 10⁻¹² M, 10⁻¹⁰ M, and 10⁻⁸ M, respectively, using a ERE-luciferase reporter (FIG. 5E). The reduced transcriptional activity of ER in AR^{-/-} MCF7 cells can be restored by transfection of np-AR (FIG. 5F). These results match well the data in FIG. 3C showing ER target gene expression is reduced in AR^{-/-} mouse breasts.

(8) The N-Terminus/DBD of AR is Required for MAPK Activation

To further determine which functional domain of AR is required to restore the normal MAPK activity in AR^{-/-} MCF7 cells, various AR fragments were reintroduced into AR^{-/-} MCF7 cells (FIG. 6A). The N-terminus together with DBD, but not ligand binding domain (LBD), LBD with deletion of helix 12 domain (LBD-dH12), DBD alone, or N-terminus alone, can restore the MAPK activation (FIG. 6A). Next, since an AR mutant (AR-R608K) has been suggested to be associated with male breast cancer (Lobaccaro et al. 1993), its effect on the MAPK activation was investigated. In AR MCF7 cells AR-R608K had a higher induction fold on MAPK activation than AR-FL (FIG. 6B), indicating that the contribution of AR-R608K to breast cancer incidence can involve the excessive activation of MAPK. Next, the requirement of AR N-terminus/DBD, but not LBD, to restore the MAPK activation was further confirmed by utilizing a double mutation AR (AR-R614H-dprm) with a deletion of the proline-rich motif (dprm) at the N-terminal region (Migliaccio et al. 2000) and a point mutation on the second zinc-finger motif (R614H) at the DBD (Beitel et al. 1994). While AR with either single mutation, AR-R614H or AR-dprm, still partially retains the ability to activate MAPK, the double mutation of AR-R614Hdprm almost loses the whole capacity to restore the MAPK activity even though these AR mutants contains intact LBDs (FIG. 6B). Finally, an attempt to restore the defect of cell proliferation by transfection of AR-FL into AR^{-/-} MCF7 cells was made. Transient transfection using expression plasmids may involve many unpredictable artificial side effects, for example, either over-expression or under-expression of AR that may result in the different influence on cell proliferation (Maucher and von Angerer 1993; Di Monaco et al. 1995; Szelei et al. 1997). However, the early results from transient transfection indicate that adding AR-FL, but not AR-R614H-dprm can partially restore the retarded cell proliferation in the AR^{-/-} MCF7. Further extensive approaches with stable transfection, using various expression vectors with the natural AR promoter, to restore the original physiological concentrations of AR in AR MCF7 cells can farther strengthen the data from lockout AR approaches and prove the essential roles of AR in MCF7 cell proliferation.

Migliaccio et al (Migliaccio et al. 2000) reported that AR could activate MAPK via the interaction between the proline-rich motif of AR and the SH3 domain of c-Src. But they demonstrated that this interaction occurred quickly and could be initiated in a short time by androgen or estrogen treatment. In contrast, FIG. 6A demonstrates that AR N-terminus/DBD, without the LBD, can induce MAPK activity, indicating that AR, but not androgen, is the major factor to activate MAPK activity. Early studies of androgen/AR roles in breast cancer are mainly focused on the effect of androgen treatment on the breast cancer: androgen could either promote or suppress breast cancer growth (Birrell et al. 1995; Xie et al. 1999; Dimitrakakis et al. 2002). Disclosed herein, however, the effect of AR protein itself can also have an effect and could go through interaction with other protein (s) to have non-genomic and/or non-androgenic activities. AR signals can utilize multiple pathways, including the classic androgen/AR→AR target genes of genomic actions as well as ARK AR interaction proteins of non-genomic action to exert its roles in the breast cancer progression. This is in agreement with early reports showing ER could also cross-talk to MAPK in breast cancer cells (Kato et al. 1995; Greene 2003). In addition to estrogens, ER could be activated via phosphorylation at Ser118 by MAPK to induce its target gene expression (Kato et al. 1995). In return, ER could also induce the Ras-Raf-MAPK cascade via non-genomic action (Migliaccio et al. 2000). The results disclosed herein show that AR can influence both MAPK and ER signals, and therefore indicates that the reduction of ER activity can be due to the reduced MAPK activity and the reduced MAPK activity can be due to the reduced ER activity in AR^{-/-} MCF7 cells and in AR^{-/-} mice.

Disclosed herein is in vivo evidence showing AR can go through growth factors, MAPK, and ER/PR signals (summary in FIG. 6C) to control the normal breast development, and modulate the breast cancer proliferation, especially in the conditions of absence of or lower E2 (FIG. 4E). Supportively, the epidemiological studies suggest that AR expression is more significantly associated with breast cancer in postmenopausal women than premenopausal women (Lea et al. 1989; Bieche et al. 2001; Honma et al. 2003), and up to the 50% of the AR-positive breast cancers are ER- and/or PR-negative (Bieche et al. 2001; Brys et al. 2002). Finally, as AR N-terminus/DBD, but not LBD, can play essential roles to modulate the growth factor signaling pathways in breast cancer cells, targeting the function of AR N-terminus/DBD represents a therapeutic approach to battle against breast cancer.

### 2. Example 2 ARA67 Functions as a Repressor to Suppress Androgen Receptor Transactivation

### a) Materials and Methods

(1) Plasmids.

The full length open reading frame (ORF) cDNA of ARA67 was generated by polymerse chain reaction (PCR) using human testis cDNA library (Clontech) as template, and subsequently constructed into pGEMT easy vector. pM-ARA67, pSG5-ARA67, and pcDNA4-ARA67 (expressing His-tagged ARA67) were constructed by releasing ARA67 from pGEMT-ARA67 with proper enzymes and inserted to the target vectors. HA-ARA67 constructs (in pKH3 vectors) and GST-ARA67 (in pGEX vectors) constructs were generated using pGEMT-ARA67 as PCR template and PCR products were subsequently digested and ligated to their target vectors. The correct constructions and expression of these plasmid constructs were verified by sequencing, TNT in vitro expression, or western blotting.

(2) Yeast Two-Hybrid Screening.

The CytoTrap Sos system (Stratagene) was used for the screening. The pSos-ARN containing the hSos gene fused with cDNA encoding ARN (amino acid 1 to 537) was generated as bait to screen a human prostate cDNA library constructed in pMyr vector (Stratagene), which expresses library proteins fused with a myristylation membrane localization signal. Expression of the myristylation sequence-tagged proteins is induced by galactose, but not glucose, and the expressed proteins are anchored to the cell membrane. The screening was carried out by co-transforming the pSos-ARN bait construct and library plasmids into a temperature-sensitive mutant yeast strain cdc25H that can not grow at a stringent temperature of 37° C. Once the bait protein physically interacts with the target protein, the hSos protein fused to the bait is recruited to the membrane, which subsequently activates the Ras signaling pathway allowing the mutant yeast strain to grow at 37° C.

(3) Cell Culture and Transfection.

H1299 and COS-1 cells were maintained in Dulbecco's modified Eagle's medium (DMEM) (Life Technologies, Inc.) supplemented with 10% heat inactivated fetal bovine serum (FBS). LNCap cells were maintained in RPMI 1640 supplemented with 10% PBS. All media contain 100 units penicillin, 100 μg/ml streptomycin and 2 mM L-glutamine. Cells were seeded to a density of 50-60% confluency for transfection. In transfections where H1299 and COS-1 cells were used, the calcium phosphate precipitation method was used as described (Pan H J, et al. 1999. Endocrine 11:321-327) unless otherwise noted. In mammalian two-hybrid assay, which did not require ligand treatment, luciferase activity of reporter gene was assayed 20-24 h after transfection using the dual-luciferase reporter assay system (Promega). In other reporter gene assays, where ligand treatment was required, cell culture media were changed to DMEM containing 10% charcoal-dextran-stripped FBS (CD-FBS) 2 h before transfection. After 16-18 h transfection, cells were treated with medium containing either vehicle or ligands for another 20-24 h and then cells were harvested. With LNCap cells transfection, the cells were treated with fresh RPMI 1640 containing 10% CD-FBS before transfection with SuperFect performed according to the manufacturer's protocol (Qiagen). After transfection, cells were allowed to recover in fresh RPMI 1640 containing 10% CD-FBS for 8-12 h, and then treated with either vehicle or ligands for another 20-24 h before harvesting. Each experiment was repeated at least three times.

(4) Glutathione S-Transferase (GST) Pull-Down Assay.

GST-ARN, GST-ARA67 fusion proteins, and GST control protein were expressed in BL21 (DE3)pLysS bacteria strain (Stratagene) and purified with glutathione-Sepharose beads as instructed by the manufacturer (Amersham Pharmacia). In vitro [³⁵S]methionine-labeled AR, ARN, AR DBD, AR LBD and ARA67 proteins were generated using TNT-coupled Reticulocyte Lysate Systems (Promega). For in vitro interaction, mixtures of glutathione beads bound GST fusion proteins and 5 μl [³⁵S]methionine-labeled input proteins in 100 μl interaction buffer (20 mM Tris/pH8.0, 60 mM NaCl, 6 mM MgCl₂, 1 mM EDTA, 0.05% Nonidet P-40, 1 mM DTT, 8% glycerol, 1 mM PMSF) were incubated in the presence or absence of 10 μM dihydrotestosterone (DHT) on a rotating disk at 4° C. for 2 h. After washing with NETN buffer (20 mM Tris/pH8.0, 100 mM NaCl, 6 mM MgCl₂, 1 mM EDTA, 0.5% Nonidet P-40, 1 mM DTT, 8% glycerol, 1 mM PMSF) four times, the bound proteins were separated on 8-13% SDS-PAGE and visualized by autoradiography.

(5) Northern Blotting

In multiple-tissue Northern blotting, the Human MTN Blot (Clontech, catalog #7760-1) was hybridized with a ³²P-labeled cDNA probe covering amino acid residues 8-140 of ARA67. The blot was subsequently probed with a β-actin cDNA probe. In cell line Northern blot, total RNA was extracted from 13 cultured cell lines as indicated using TRIZOL reagent (GIBCO) and 20 μg of total RNA was transferred onto Nylon membrane for Northern blotting. The RNA bound membrane was hybridized with the same ARA67 cDNA probe as described above. 18S RNA was used as RNA loading control.

(6) Western Blotting

Protein samples collected from the cells were separated on 8% SDS-PAGE and transferred to nitrocellulose membranes. Membranes were blocked with 5% nonfat milk in TBST buffer (150 mM NaCl, 10 mM Tris/pH8.0, and 0.5% Tween-20) at room temperature for 1 h. Then the membranes were immunoblotted with primary antibodies for 2 h at room temperature or overnight at 4° C., followed by incubation with secondary antibodies for 1 h at room temperature. Blots were developed with the AP color developing reagents (Bio-Rad).

(7) Coimmunoprecipitation Assay

COS-1 cells seeded on 100 mm cell culture dishes were transient transfected with AR and HA-ARA67 expression plasmids in combinations as noted in FIG. 9C, using SuperFect transfection reagent (Qiagen) following company protocols. Other transfection and treatment procedures were the same as described herein. Cells were harvested and dissolved in RIPA buffer (1×PBS, 0.5% Nonidet P-40, 0.5% sodium deoxycholate, 0.05% SDS, 1 mM PMSF, 1× protease inhibitor cocktail (Roche)). Cell lysates containing 500 μg proteins were precleared with 20 μl protein A/G PLUS-agarose (Santa Cruz Biotechnology) for 0.5 h. The supernatant was then mixed with 5 μg/ml mouse monocolonal anti-AR antibody (BD Pharmingen, catalog #554226) at 4° C. for 2 h, followed by adding protein A/G PLUS-agarose and mixing for another 2 h. Immunoprecipitates obtained by spinning down protein A/G PLUS-agarose were washed with PBS for 3 times and separated on 8% SDS-PAGE. The results were analyzed by Western blotting as described above.

(8) Immunofluorescence Staining

COS-1 cells were seeded on two-well Lab Tek Chamber slides (Nalge) in DMEM containing 10% CD-FBS 18 h before transfection. DNA was transfected by using FuGENE 6 Transfection Reagent (Boehringer Mannheim). After transfection, cells were treated with either 10 nM DHT or vehicle for 12 h. Then cells were fixed with fixation solution (3% paraformaldehyde and 10% sucrose in PBS) for 20 min on ice, followed by permeabilization with methanol for 10 min on ice. Slides were washed and blocked with 2% BSA in PBS for 15 min at room temperature. Then the cells were stained with 1 μg/ml rabbit polyclonal anti-AR antibody (NH27) and 1 μg/ml mouse monoclonal anti-His antibody (Santa Cruz Biotechnology) sequentially at room temperature for 1 h each. After each first Ab incubation, cells were washed and incubated with TexRed-conjugated goat anti-rabbit IgG and FITC-conjugated goat anti-mouse IgG (ICN), respectively. Stained slides were washed and mounted (Vectashield; Vector Laboratories). Fluorescence images were photographed under 400-fold magnification with a confocal microscope.

(9) Subcellular Fractionation

To prepare cytosolic and nuclear fractions of cells, cell monolayers were harvested with ice-cold PBS and pelleted. Cold buffer A (10 mM HEPES-KOH/pH 7.9 at 4° C., 1.5 mM MgCl₂, 10 mM KCl, 0.5 mM dithiothreitol, 0.2 mM PMSF) equal to 5 times cell volume was used to resuspend the cells. After swelling on ice for 10 min, plasma membranes were disrupted by vortexing for 10 sec. The nuclei were pelleted by centrifugation at 12,000 rpm for 20 sec at room temperature. Supernatants containing the cytosolic fraction of proteins were recovered. The remaining pellets were resuspended in 20-50 μl cold buffer C (20 mM HEPES-KOH/pH 7.9, 25% glycerol, 420 mM NaCl, 1.5 mM MgCl₂, 0.2 mM EDTA, 0.5 mM dithiothreitol, 0.2 mM PMSF) and incubated on ice for 20 min. Samples were then centrifuged at 12,000 rpm for 2 min at 4° C. to remove the cellular debris. The supernatants containing the nuclear fraction were recovered.

### b) Results

(1) Identification of ARA67 as an AR N-Terminal Interacting Protein.

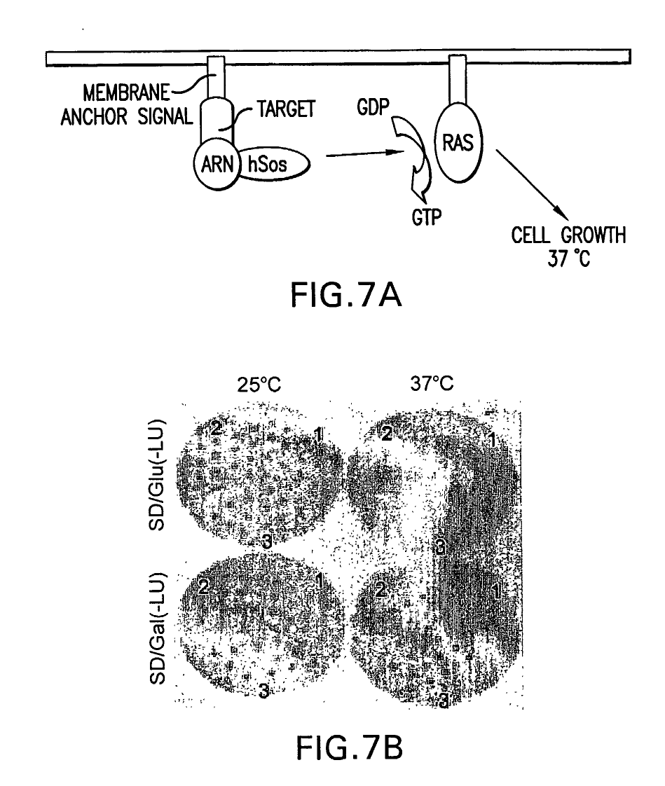

To identify ARN interacting proteins, the CytoTrap Sos system (Stratagene) was selected for the screening, since the ARN contains the AF-1 which can be self-transactive, making it hard to be used as a bait in the conventional yeast two-hybrid screening. The CytoTrap Sos system is based on generating fusion proteins whose interaction in the yeast cytoplasm induces cell growth by activating the Ras signaling pathway (FIG. 7A), which is advantageous over the conventional yeast two-hybrid system in screening interacting proteins for transcriptional activators. In this approach, cDNA encoding ARN (amino acid 1 to 537) was constructed into the pSos vector as bait to screen a human prostate cDNA library. The bait and library constructs were co-transformed into yeast strain cdc25H. Of 8×10⁵ clones screened, 2 positive clones were identified. One of the clones named ARA67 matched a DNA sequence encoding amino acid 20-585 of the protein PAT1 (SEQ ID NO:1) (Zheng, P. et al. 1998. Proc. Natl. Acad. Sci. USA 95:14745-14750) (GenBank accession no. AF017782) at an identity of 99.6%. 5′ rapid amplification of cDNA ends was used to obtain the full length ARA67, which contains an open reading frame encoding 585 amino acids that matches the reported PAT1 sequence (Zheng, P. et al. 1998. Proc. Natl. Acad. Sci. USA 95:14745-14750). The interaction in yeast was confirmed by re-transforming the pMyr-ARA67 into yeast cells pre-transformed with pSos-ARN and allowing the transformants to grow on synthetic drop-out (SD) glucose agar lacking leucine and uracil [SD/Glu (−LU)] and SD galactose agar lacking leucine and uracil [SD/Gal (−LU)] plates at the stringent temperature of 37° C. Only the clones that grow out on SD/Gal (−LU) plates but not on SD/Glu (−LU) plates at 37° C. are interaction positive ones. When pSos-ARN and pMyr-ARA67 co-exist, the clones showed positive growth (FIG. 7B).

(2) ARA67 Selectively Binds to ARN

To test if the interaction between ARA67 and ARN was specific, pMyr-ARA67 was co-transformed with several other pSos constructs, including testicular receptor 2 (TR2) (SEQ ID NO:13) (Chang, C., et al. 1988. Biochem. Biophys. Res. Commun. 155:971-977), testicular receptor 4 (TR4) (SEQ ID NO:14) (Chang, C., et al. 1994. Proc. Natl. Acad. Sci. USA 91:6040-6044), ARA55 (Fujimoto, N., et al. 1999. J. Biol. Chem. 274:8316-8321), ARA70 (Yeh, S. et al. 1996. Proc. Natl. Acad. Sci. USA 93: 5517-5521), and two control plasmids (pSos-MAFB and pSos-Coll) provided by the company, into the temperature-sensitive mutant yeast strain. After selection, only those yeast cells that contained both ARN and ARA67 expression plasmids showed positive growth, while yeast cells contained all other pairs of plasmids couldn't grow on SD/Gal (−LU) plate at 37° C. (summarized in table 3). In Table 3. ARA67 selectively binds to ARN in yeast. pMyr-ARA67 was co-transformed with several other pSos-fusion protein constructs. The table summarizes the results. As shown, only ARN interacted with ARA67 allowing the yeast host to grow at the stringent temperature of 37° C., while other proteins tested could not. These data showed that ARA67 interacts with ARN and the interaction is rather selective.

(3) Distribution of ARA67 in Human Tissues and Multiple Cell Lines

To detect the expression of ARA67 in human tissues and cell lines, Northern blot analysis was performed with a probe covering amino acid residues 8 to 140. Three transcripts with the sizes of 2.5 kb, 4.4 kb and 7.5 kb were detected (FIG. 8). ARA67 was widely expressed in multiple human tissues at variable levels. Strong expressions of all three transcripts were seen in heart, placenta and skeletal muscle, while in other tissues, moderate to low expression levels were detected (FIG. 8A). The three ARA67 transcripts were also seen in all the cell lines tested with the 4.4 kb transcript having the highest expression level. Among these cell lines, LNCaP, DU145, PC-3, and RPWE-1 originated from prostate, MCF-7, MDAMB231, and T47D from breast, GC-SPG and Tm4 from testis, H1299 from lung, HepG2 from liver, HTB14 from brain, and COS-1 from monkey kidney. An overexpression was seen in MCF-7. The different sizes of the transcripts can result from alternative splicing or be due to the inclusion of different lengths of untranslated regions. Whether the three transcripts represent three different protein products remains to be answered. However, the relative transcription levels of the three transcripts are not consistent among different tissues and cell lines, indicating that regulation of the gene products of ARA67 can be required for maintaining different characteristics or functions of the cells.

(4) Interaction of ARA67 and AR In Vitro and In Vivo.

To confirm the interaction between ARA67 and ARN seen in the yeast two-hybrid assay, ARA67 and ARN cDNA were constructed into pM and pVP16 vectors (Clontech) for assaying in a mammalian two-hybrid system. As seen in FIG. 9A, the reporter luciferase activity was highly induced in cells co-transformed with pM-ARA67 and pVP16-ARN, indicating a strong association between ARA67 and ARN. To prove that ARA67 and AR directly interact, in vitro GST pull down assay was carried out. The data show ARA67 interacted with AR in a DHT independent manner (FIG. 9B). When separating AR into ARN, ARDBD and ARLBD, all three fragments could interact with ARA67 but with different strengths. ARA67 interacted with ARN most strongly, ARLBD moderately, and ARDBD very weakly (FIG. 9B). The association between ARA67 and AR is also revealed by co-immunoprecipitation assay. Cell lysates from COS-1 cells transfected with either AR or AR with HA-ARA67 were immunoprecipitated with anti-AR antibody. The immunoprecipitates were analyzed by Western blotting. As shown in FIG. 9C, ARA67 was detected in AR containing complex either in the presence or absence of DHT (lane 3 and 4), while ARA67 was not detected in negative control lanes (lane 1, 2 and 5). Together, results from GST pull-down (FIG. 9B), yeast two-hybrid (FIG. 7B), mammalian two-hybrid (FIG. 9A), and co-immunoprecipitation (FIG. 9C) assays all prove that ARA67 and AR interact in vitro and in vivo.

(5) ARA67 Suppresses AR Transactivation Activity.

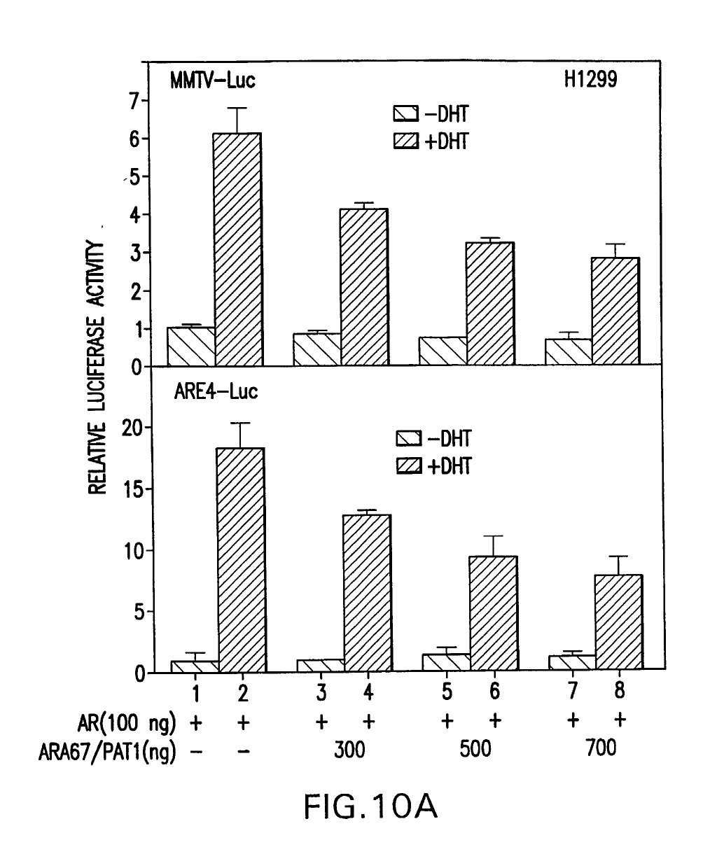

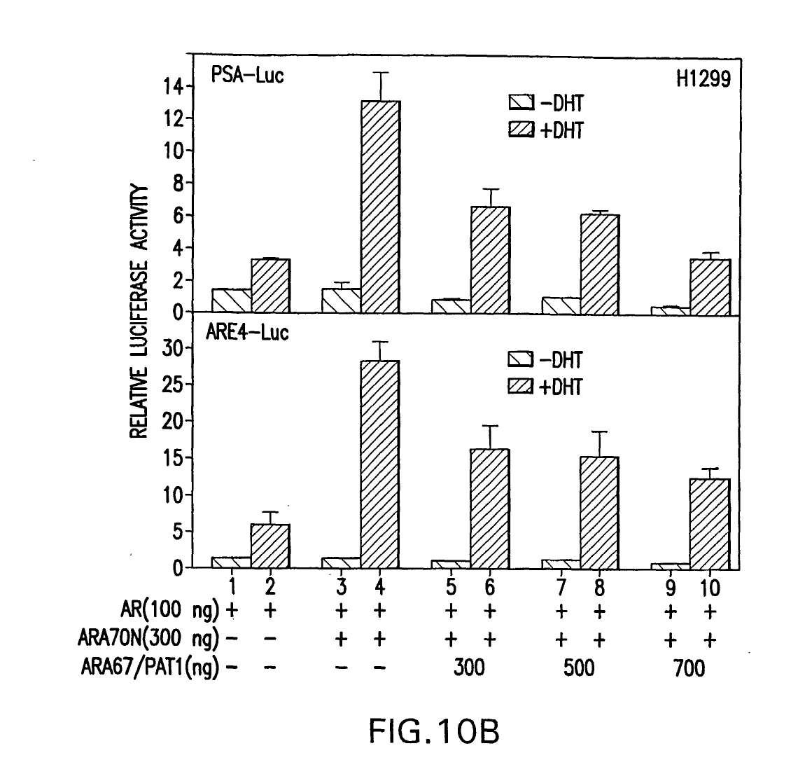

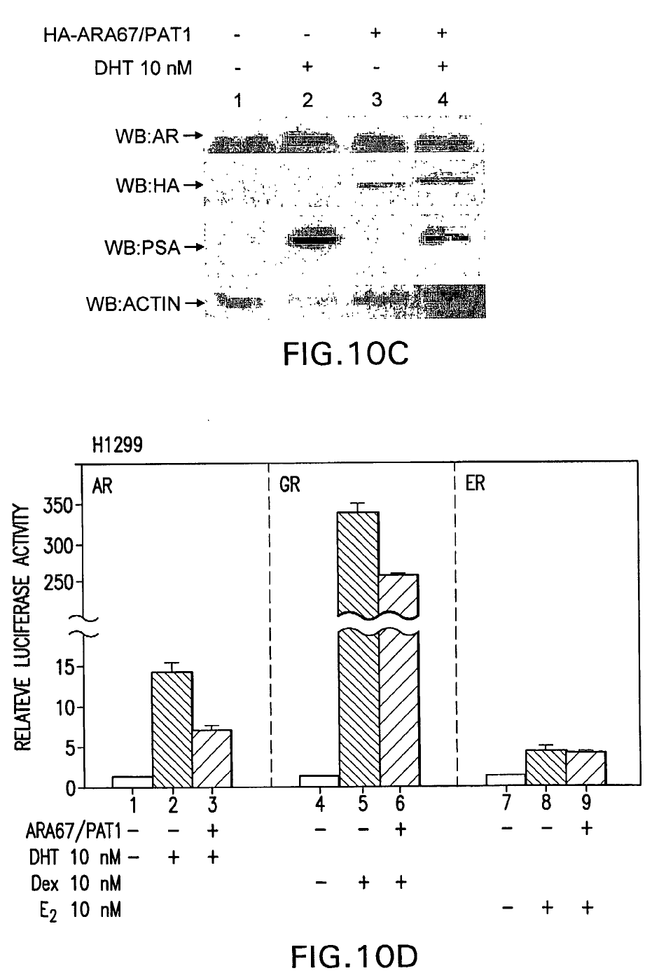

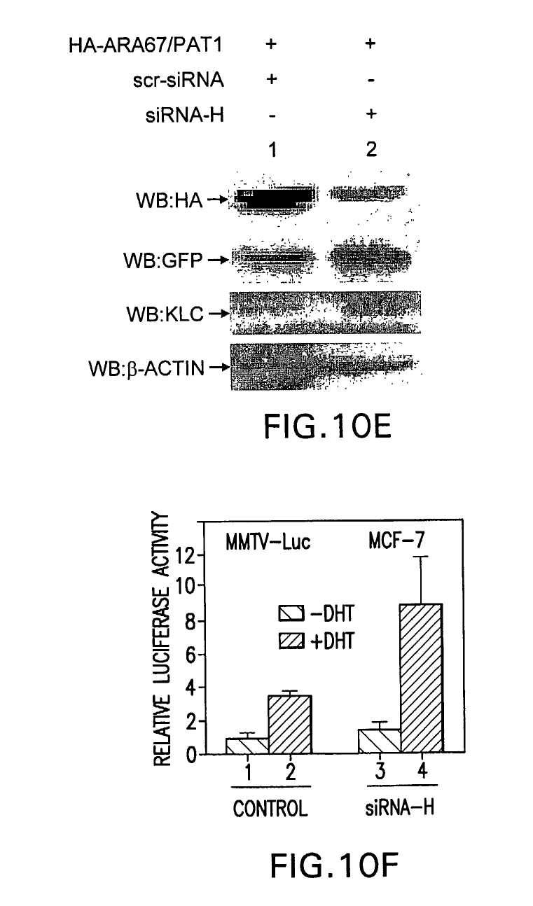

To test whether ARA67 can influence AR function, reporter gene assays were performed. As shown in FIG. 10A, in H1299 cells ARA67 suppressed DHT-induced AR transactivation dose-dependently with MMTV-Luc and ARE₄-Luc as reporters. It was then asked whether ARA67 could also counteract coactivator-enhanced AR transactivation. ARA70 N-terminus (ARA70N) (Yeh, S. et al. 1996. Proc. Natl. Acad. Sci. USA 93: 5517-5521), a potent AR coactivator, was chosen for the experiments. As shown in FIG. 10B, when co-transfected with AR, ARA70N significantly enhanced DHT-induced AR transactivation. While in the presence of ARA67, ARA70N enhanced AR transactivation was repressed dose-dependently with PSA-Luc and ARE₄-Luc as reporters. To further prove that AR function is suppressed by ARA67, whether ARA67 could influence the expression of AR target gene prostate specific antigen (PSA) in LNCaP cells was tested. As shown in FIG. 10C, when ARA67 was transfected into the cells, the DHT induced PSA expression was decreased. To test whether the transcriptional suppression of ARA67 is a general effect with nuclear receptors or more specific to AR, AR, GR and ER transactivation were compared in the presence of ARA67. At the same dose, ARA67 showed the most significant suppression on AR activity (50%), slight suppression on GR (20%), and little effect on ER (FIG. 10D), indicating the suppression on AR is selective. Together, the data in FIG. 10A-D show ARA67 functions as a suppressor to AR and the suppression is relatively more selective to AR.

(6) Interaction Domains Between ARA67 and AR and their Influence on AR Transactivation.

Since the data already showed that ARN interacts with ARA67 strongly, the next step was to determine which part of ARN is important for the interaction. Different GST-ARN-fragment fusion protein constructs were generated and expressed (FIG. 11A). After incubation with in vitro [³⁵S]methionine-labeled ARA67, only ARN₁₋₁₄₀ showed positive interaction although not as strong as that seen in ARN full length (ARN₁₋₅₅₆) (FIG. 11A). These data indicate residues 1-140 within ARN are critical for the interaction with ARA67. Since important regions for AR transactivation within ARN are in residues 141-338, which are required for full ligand-inducible transcription, and residues 360-494, which contain the AF-1 region that is also required for full AR function (Heinlein, C. A., et al. 2002. Endocr. Rev. 23:175-200), the data showing that AR residues 1-140 interact with ARA67 indicated that a different domain within ARN can be involved in ARA67 mediated suppression on AR transactivation.

Motif scan indicated several protein-protein interaction motif/domains (including leucine zipper and LXXLL motif) existed in ARA67/PAT1. Several truncated ARA67 fragments were constructed (FIG. 11B) to see which part plays a key role for the interaction with AR. A GST pull-down assay was performed. The results showed that both the N-terminal (ARA67₁₋₂₈₀) and C-terminal (ARA67₂₈₁₋₅₈₅) regions of ARA67 can interact with ARN but the interaction is relatively weak. ARA67₈₋₁₄₀ and ARA67₂₈₁₋₅₅₀ showed slightly stronger interaction with ARN than their bigger counterparts ARA67₁₋₂₈₀ and ARA67₂₈₁-585, respectively, while ARA67₂₈₁-550 was better than ARA67₈₋₁₄₀. Although no fragment constructs of ARA67 strongly interacted with ARN, full length ARA67 showed strong interaction with ARN, indicating participation of different parts of ARA67 can be required for the interaction (FIG. 11B). The interaction pattern between ARA67 fragments and AR LBD was similar to that between ARA67 fragments and ARN, but the ARA67 C-terminal fragment showed an interaction strength similar to full length ARA67 (FIG. 11C) with the LBD, which indicates that the interaction between ARA67 and AR LBD may not need the cooperation of the N- and C-termini of ARA67. Amino acid sequences located within 8-140 and 339-550 of ARA67 can contribute more to its interaction with AR (FIGS. 11B, 11C). ARA67 contains a LXXLL motif, which is a signature motif in many NR coactivators that is important for their binding to NRs (Heery, D. M., et al. Nature 387:733-736). In the data, GST-ARA67₁₇₀₋₃₃₈, which contains the LXXLL motif showed very weak interaction with ARN and no interaction with AR LBD, indicating the LXXLL motif in ARA67 is not critical for the interaction with AR.

Then it was determined if there is any domain or sequence that is essential for ARA67 to suppress AR. Several truncated ARA67 fragments were constructed and their influences on AR transactivation were tested using reporter gene assay. ARA67 contains a PEST sequence at its C-terminal end (Gao, Y., et al. 2001. Proc. Natl. Acad. Sci. USA 98:14979-14984), which is often seen in regulatory proteins with high turnover rate. ARA67 lacking the PEST sequence (ARA67₁₋₅₅₀) may be more stabilized and be more potent as an AR repressor, but as seen in FIG. 11D, ARA67₁₋₅₅₀ didn't show a stronger suppression effect than full length ARA67. Western blot was also performed to test the expression of ARA67 fragment constructs and found the protein level of ARA67₁₋₅₅₀ was similar to that of ARA67₁₋₅₈₅ 24 h after transfection. ARA67 also contains nuclear localization signals (NLSs) at its C-terminus and ARA67/PAT1₁₋₄₁₁, a truncated form lacking the NLSs, remains in cytosol and can not enter nucleus (Gao, Y., et al. 2001. Proc. Natl. Acad. Sci. USA 498:14979-14984). Whether the nuclear localization of ARA67 is required for its suppression on AR was then tested. As shown in FIG. 11D, ARA67₁₋₄₁₁ had similar suppression effect as fall length ARA67 did, indicating that the nuclear localization of ARA67 is not critical for its effect on AR. The N-terminal (ARA671-280) and C-terminal (ARA67₂₈₁₋₅₅₀, ARA67₂₈₁₋₅₈₅) regions of ARA67 could also suppress AR transactivation, however ARA67₁₋₂₈₀ was better suppressor than ARA67₂₈₁₋₅₅₀ and ARA67₂₈₁₋₅₈₅Together FIG. 11B-D show that both the N- and C-terminal regions of ARA67 are involved in the interaction with and suppression of AR, and the interaction strength is not the sole determinant of suppression potency.

(7) The influence of ARA67 on AR N-/C-termini (N/C) interaction.

Early reports suggest that AR N/C interaction can stabilize androgen bound AR (Zhou, Z. X., et al. 1995. Mol. Endocrinol. 9: 208-218) and AR N-terminus is required for the full ligand-induced AR transactivation (Simental J. A., et al. 1991. J. Biol Chem. 266:510-518). Since ARA67 can interact with both AR N-terminus and AR C-terminus (FIG. 9B), it's possible that ARA67 can influence AR transactivation by blocking AR N/C interaction. Using a mammalian two-hybrid assay, it was shown that DHT promoted AR N/C interaction (FIG. 12A). When ARA67 was present, DHT promoted AR N/C interaction was slightly enhanced rather than suppressed. This is in contrast with a coactivator of AR, SRC-1, which has been reported to be able to enhance AR N/C interaction (Ikonen, T., et al. 1997. J. Biol. Chem. 272:29821-29828). Then the influence of ARA67 on AR protein level was tested. Consistent with the AR N/C interaction data, AR protein level was slightly increased rather than decreased in the presence ARA67 (FIG. 12B). Therefore, the suppression effect of ARA67 on AR cannot be explained by its influence on AR N/C interaction.

(8) Histone deacetylase (HDAc) activity is not involved in ARA67 mediated suppression effect on AR.

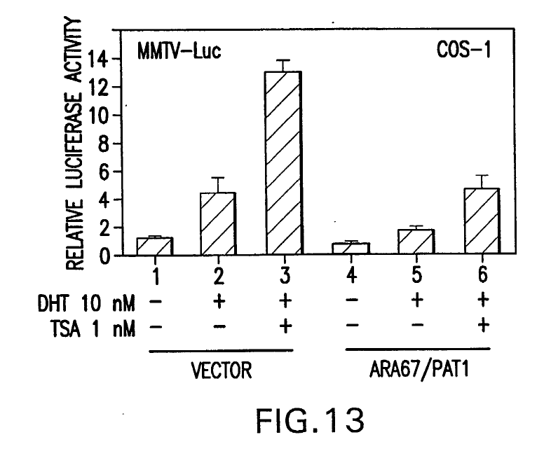

It has been suggested that coactivator and corepressor complexes, which exhibit histone transferase and histone deacetylase activities, respectively, play an important role in regulating NR transactivation activity (Xu, L., et al. 1999. Curr. Opin. Genet. Dev. 9: 140-147). AR is one of the non-histone proteins that can be acetylated and a point mutation at the acetylation site abrogates DHT-induced AR transactivation in cultured cells (Fu, M., et al. 2000. J. Biol. Chem. 275:20853-20860). In the same report, trichostatin A (TSA), a specific histone deacetylase inhibitor, was shown to enhance ligand-induced AR transactivation. ARA67 contains several putative protein-protein interaction domains and the data also show ARA67 can interact with AR through multiple sites (FIGS. 11B and 11C). It's possible that it behaves as an adapter between AR and regulatory multi-protein complexes that contain HDAc activity. To test this hypothesis the effect of TSA on ARA67 function was examined. First tested were several different TSA concentrations to assure the best working conditions. In this system, TSA at 10 nM and above caused significant cell death in COS-1 and H1299, while 1 nM TSA showed no obvious toxic effect and gave the best activation on AR. As shown in FIG. 13, 1 nM TSA enhanced DHT-induced AR transactivation. In the presence of ARA67, TSA enhanced AR transactivation was repressed to a similar extent as DHT-induced AR transactivation was repressed. The data indicate that TSA's effect and ARA67's effect on AR are parallel to each other, which indicate HDAc activity is not involved in ARA67 mediated suppression on AR.

(9) ARA67 Influences the Subcellular Distribution of AR

It's known that upon ligand binding, AR translocates from the cytosol to the nucleus where it binds to the ARE of its target gene and turns on the expression of its target gene. Decrease of AR nuclear translocation has been reported to lead to suppression of AR transactivation and androgen induced cell growth (Gerdes, M. J., et al. 1998. Endocrinology 139:3569-3577). ARA67 is present in both cytosol and nucleus (Gao, Y., et al. 2001. Proc. Natl. Acad. Sci. USA 98:14979-14984), shares homology with kinesin light chain (Zheng, P. et al. 1998. Proc. Natl. Acad. Sci. USA 95:14745-14750) that is involved in protein trafficking, and can interact with the microtubule (Zheng, P. et al. 1998. Proc. Natl. Acad. Sci. USA 95:14745-14750), a cytoskeleton structure. Thus, it's possible that ARA67 can suppress AR transactivation through interrupting AR nuclear translocation. To test this possibility, immunofluorescence staining analyses was performed with COS-1 cells co-transfected with AR and His-tagged ARA67 expression plasmids or vector and then treated with either vehicle or DHT. The subcellular localization of AR was examined as red fluorescence signal under the microscope. The data show that in the absence of ARA67, AR remained in the cytosol without DHT treatment and moved into nucleus after DHT treatment. In the presence of ARA67, without DHT the cytosolic localization of AR was not obviously influenced, but after DHT treatment, the AR signal remained mostly in the cytosol and the signal in nucleus was very weak (FIG. 14A). Therefore, ARA67 can block the nuclear translocation of AR. To support the immunofluorescence staining result, a Western blot was performed with separated cytosolic and nuclear fractions of proteins. As shown in FIG. 14B, after DHT treatment, the AR level in nuclear fraction decreased when ARA67 was cotransfected with AR. By using immunofluorescence staining and Western blot, it was demonstrated that ARA67 can inhibit AR nuclear translocation, which can be the major mechanism through which ARA67 is able to suppress AR transactivation. Disclosed herein ARA67 interacts with AR and functions as a repressor of AR. Many coregulators of AR have been identified and characterized. Compared to coactivators, the corepressors of AR identified are relatively fewer and less well characterized. Calreticulin can bind to AR DBD, and suppress AR transactivation by blocking AR binding to target DNA sequences (Burn, K., et al. 1994. Nature 367:476-480, Dedhar, S., et al. 1994. Nature 367:480-483). Cyclin D has been reported to suppress AR function presumably through influencing androgen-dependent transactivation function in ARN (Petre, C. E., et al. 2002. J. Biol. Chem. 277:2207-2215). Since androgen action involves dissociation of AR from heatshock protein complex, homodimerization, nuclear translocation, and binding to target genes, all these processes can be influenced by coregulators.

Nuclear localization of androgen bound AR is a prerequisite for its transactivation function. However, relatively little is known about the mechanism of its nucleocytoplasmic trafficking and its interacting proteins that can be involved in this process. The data show that ARA67 is able to trap AR in the cytosol, indicating that it can play a role in AR trafficking. Early studies show that ARA67/PAT1 shares homology with kinesin light chain, a molecular motor driving the trafficking of cargos along the microtubule, directly interacts with the microtubule, and is functionally related to APP trafficking/processing (Zheng, P. et al. 1998. Proc. Natl. Acad. Sci. USA 95:14745-14750). The data correlate with these findings supporting the role of ARA67 in protein trafficking. However, the detailed mechanisms need further investigation. Studies with GR suggest that an intact cytoskeleton network is required for the shuttling of GR between the cytosol and nucleus in physiological conditions (Galigniana, M. D., et al. 1998. Mol. Endocrinol. 12:1903-1913, Galigniana, M. D., et al. 1999. J. Biol. Chem. 275:16222-16227). It's not clear whether this represents a common feature in nucleocytoplasmic shuttling of SHRs, since the ligand-dependent translocation of PR has been suggested as independent of cytoskeleton integrity (Perrot-Applanat, M., et al. 1992. J. Cell Biol. 119:337-348). ARA67/PAT1 can bind microtubules and the binding can be enhanced 5-10 fold in the presence of Mg-ATP (Zheng, P. et al. 1998. Proc. Natl. Acad. Sci. USA 95:14745-14750), suggesting the possibility that the microtubule network can be an important component for ARA67 to trap AR in the cytosol. Many proteins are involved in the subcellular distribution of AR. In the absence of ligands, AR associates with the heatshock protein complex, which keep AR in an inactive state in the cytosol, while upon ligand-binding, filamin is required for AR to translocate to nucleus (Ozanne, D. M., et al. 2000. Mol. Endocrinol. 14:1618-1626).

It's known that AR N- and C-terminus can directly interact through the LXXLL like motif present in AR N-terminus and AF-2 domain in AR C-terminus (He, B., et al. 1999. J. Biol. Chem. 274:37219-37225, He, B., et al. 2000. J. Biol. Chem. 275:22986-22994). Upon ligand binding, helix 12 in AR LBD folds across the ligand binding pocket, which reduces the dissociation rate of bound androgen and helps to stabilize AR protein. AR N/C interaction stabilizes the position of helix 12 when androgen is bound to AR (Zhou, Z. X., et al. 1995. Mol. Endocrinol. 9: 208-218, He, B., et al. 1999. J. Biol. Chem. 274:37219-37225). Coregulators that influence the AR N/C interaction could affect the stability of AR and thus AR transactivation. One of the mechanisms by which coactivators enhance AR transaction is through facilitating AR N/C interactions as seen in SRC-1 and CBP mediated coactivation (Ikonen, T., et al. 1997. J. Biol. Chem. 272:29821-29828). Since ARA67 can interact with both AR N- and C-termini, it's reasonable to hypothesize that ARA67 can influence the AR N/C interaction. The results show ARA67 enhances the interaction between AR N- and C-termini, and accordingly observed was a mild increase in AR protein level that can result from an increased AR stability. These seem to be contradictory to the role of ARA67 as a corepressor. However, it was shown that ARA67 can block AR translocation to the nucleus upon AR-ligand binding. This can prevent increased AR transactivation resulting from elevated AR protein levels, since only nucleus localized AR can exert its influence on its target genes. Because these two opposite factors co-exist, it's possible that the cellular context can influence the net outcome. ARA67 contains several hypothetical protein kinase C phosphorylation sites (Zheng, P. et al. 1998. Proc. Natl. Acad. Sci. USA 95:14745-14750), suggesting the possibility that ARA67's activity is under the influence of certain cell signaling. The potential of ARA67 as corepressor can differ among different cells, since subcellular environments can vary. It could be assumed that certain modifications on ARA67 may weaken its ability to block AR nuclear translocation, while the increased AR protein level may be dominant, in which case ARA67 can function as a coactivator rather than corepressor of AR.

In summary, it was demonstrated that ARA67 can interact with AR and suppress AR transactivation. The major mechanism for ARA67 to function as a repressor is through interrupting AR nuclear translocation. In addition, ARA67 has the potential to enhance AR transactivation through enhancing AR N/C interaction and AR stability. Since AR is one of the key players in prostate carcinogenesis, it's possible that some of the prostate cancer cells can take advantage of the potential function of ARA67 as coactivator of AR by altering the cellular environment to inhibit its corepressor's function. Further study can provide more insight into the development and progression of prostate cancer. Furthermore, ARA67 can also bind specifically to APP (Zheng, P. et al. 1998. Proc. Natl. Acad. Sci. USA 95:14745-14750), a protein that is involved in the pathogenesis of Alzheimer's disease. Testosterone has been reported to protect neurons from neurotoxic damage through the AR-mediated pathway (Hammond, J., et al. 2001. J. Neruochem. 77:1319-1326). AR may play a role in neuron related diseases through the linkage of ARA67.

### 3. Example 3

### a) Materials and Methods

(1) Materials and Plasmids.

5u-Dihydrotestosterone (DHT) and Lithium Chloride (LiCl) were obtained from Sigma. Antibodies to GSK3β and phospho-GSK3β were purchased from New England Biolabs. Purified GSK3β was purchased from Upstate Biotechnology, Lake Placid, N.Y. The anti-AR polyclonal antibody, NH27, was produced as described (Yeh, S., et al. (1996)Proc Natl Acad Sci USA 93 (11), 5517-21). The GSK3β plasmids, including wild type, constitutively active, and dominant negative forms, were kindly provided by J. Sadoshima, Pennsylvania State University. (“The Akt-glycogen synthase Kinase 3 Beta Pathway Regulates Transcription of Atrial Natriuretic Factor Induced by Beta-Adrenergic Receptor Stimulation in Cardiac Myocytes by Carmine Morisco, David Zebrowski, Gianluigi Condorelli, Philip Tsichlis, Stephen F. Vatner, and Junichi Sadoshiima JBC, 275, 14466-14475, 2000, which is herein incorporated vy reference at least for material related to GSK3B plasmids)

(2) Cell Culture and Transfection Assay.

COS-1 and PC-3 cells were maintained in early to mid-log phase in DMEM medium, supplemented with 10% fetal bovine serum (FBS), 50 units/ml penicillin and 50 μg/ml streptormnycin in incubators with humidified air and 5% carbon dioxide at 37° C. LNCaP cells were maintained in RPMI (GIBCO/BRL) medium. Twenty-four h prior to transfection, cells washed with Hanks' buffered saline solution, trypsinized, and seeded to be a density of 40-60% confluence for transfection. Cells in 12 well plates were refed with fresh medium 2 hours before transfection and transfected according to the “SuperFect Transfection” instructions (QIAGEN). After 2-3 h incubation, cells were treated with medium supplemented with charcoal-dextran treated FBS containing either ethanol or ligands. Cells were further incubated at 37° C. for 24 h, washed with PBS, and harvested.

(3) In Vitro Kinase Assay.

Purified recombinant, murine MAP kinase (New England Biolabs) was assayed as described (Yeh, S., et al. (1999) Proc Natl Acad Sci USA 96 (10), 5458-63). The kinase buffer contains 25 mM HEPES, pH 7.4, 10 mM MgCl2, and 1 mM dithiothreitol). The kinase reactions were performed for 30 min at 30° C. in the presence of 10 μCi [−³²P]ATP, 10 μM ATP, and 0.05 pmol of GSK3β. The reactions were terminated by addition of 4×SDS sample buffer. The samples were boiled and loaded on 12% SDS-polyacrylamide gel electrophoresis gels.

(4) Stable S9A-GSK3β Transfection in CWR22R Cell.

The S9A-GSK3β gene was inserted into pBig vector with hygromycin resistance. The S9A-GSK3β-transfected CWR22R cells were selected and maintained in RPMI medium containing 50 μg/ml hygromycin (GIBCO).

(5) Thiazolyl Blue (MTT) Assay.

The MTT assay is a quantitative colorimetric assay for mammalian cell survival and proliferation. The 5×10³ CWR22R cells were seeded in 24-well plates and incubated in RPMI medium 1640 with 5% CS-FCS for 48 h. Cells were then treated with ethanol, 10 nMDHT, and/or 2 μg/ml doxycycline for another 5 days. Then 200 μl of MTT (5 mg/ml; Sigma) was added into the each well with 1 ml of medium for 3 h at 37° C. After incubation, 2 ml of 0.04 M HCl in isopropyl alcohol was added into each well. After 5-min incubation at room temperature, the absorbance was read at a test wavelength of 570 nm.

### b) Results

(1) GSK3β is ubiquitously expressed in prostate cancer cells.

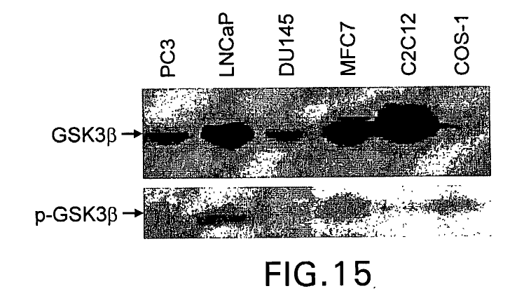

Early studies showed that GSK3β mRNA was prominently expressed in testis, thymus, prostate, and ovary (Lau, K. F., et al. (1999) JPept Res 54 (1), 85-91). To examine the protein expression and activity of endogenous GSK3β in prostate cancer cells, several prostate cancer cell lines, including PC-3, LNCaP, and DU145, were subjected to Western blotting analysis along with some non-prostate cancer cell lines, including MCF7, C2C12, and COS-1. GSK3β was ubiquitously expressed in all cell lines analyzed. Furthermore, the phosphorylation status at serine-9 of GSK3β was determined by Western blotting of the cell lysate with phospho-specific antibodies. LNCaP cells showed strongly phosphorylated GSK3β compared with PC-3 and DU145 cells (FIG. 15), indicating lower endogenous activity of GSK3β in LNCaP cells.

(2) Suppression of AR Transaetivation by GSK3β.

Since growth factors, neuropeptides and protein kinase A inhibit GSK3β and enhance AR activity concurrently (Sadar, M. D. (1999) J Biol Chem 274 (12), 7777-83, Lee, L. F., et al. (2001) Mol Cell Biol 21 (24), 8385-97, Culig, Z., et al. (1994) Cancer Res 54 (20), 5474-8, Shaw, M., et al. (1997) FEBSLett 416 (3), 307-11, Woodgett, J. R. (2001) Sci STKE 2001 (100), RE12), it was of interest to see whether co-expression of GSK3β might alter AR-dependent transcriptional activity. Advantage was taken of a dual luciferase assay system (Promega) using reporter and internal control plasmids together. The ARE4-Luc reporter is driven by four androgen response elements (ARE) in the promoter region, and functions as a monitor of AR transcriptional activity. Renilla luciferase is driven by the SV-40 promoter and serves as an internal control for transfection efficiency. GSK3β, AR, and the two reporter plasmids were transiently co-transfected in COS-1 cells, which lack endogenous AR. As shown in FIG. 16A, wild type (WT) GSK3β reduced the AR-mediated transcription of the luciferase reporter by about 40% (lanes 2). While inactive GSK3β (KM-GSK3β had only a maginal effect on AR, the constitutively active form of the GSK39 (S9A-GSK3β) strongly inhibited AR activity (lane 4, and 5), indicating that the kinase activity of GSK3β is necessary to suppress AR activity.

Since the context of upstream promoter elements may influence transcriptional efficiency, another reporter plasmid, MMTV-Luc, was tested to confirm the suppression effect of GSK3β on AR transcriptional activity. MMTV-Luc is driven by the natural MMTV-LTR promoter that contains several AR response elements. FIG. 16B demonstrates that GSK3β inhibits DHT-mediated AR transactivation in a dose-dependent manner (lanes 2-5). Lithium Chloride (LiCl), a specific inhibitor of GSK3β, not only abolished the inhibitory effect of GSK3β on AR, but also slightly enhanced AR transcriptional activity. This result indicates that LiCl can block both exogenously transfected GSK3β as well as the endogenous GSK3β activity in COS-1 cells. Moreover, LiCl did not alter luciferase expression in the absence of AR, ensuring that LiCl has no non-specific effect on the MMTV-Luc reporter. To rule out the possibility that GSK3β may have nonspecific effects on the general transcription machinery, also tested was its effect on the human glucocorticoid receptor (hGR) since early studies reported GSK3β has little effect on the phosphorylation of hGR. As shown in FIG. 16C, addition of GSK3β failed to inhibit hGR transactivation. Together, results from FIG. 16A to 16C indicate that GSK3β can selectively inhibit AR transactivation.

Unlike many other kinases, GSK3β is constitutively active in most tissues. Seeing if endogenous GSK3β inhibits AR-mediated transcription was of interest. The human prostate cancer cell line PC-3 is an AR-negative cell line that contains active endogenous GSK3β. PC-3 cells were transiently transfected with AR, the MMTV-Luc reporter and internal control plasmids. As shown in FIG. 16D, LiCl has marginal effects on basal level of AR transactivation in COS-1 cells (lanes 1-3) in the absence of androgen. Since PC-3 cells contain more total GSK3β and less phosphorylated GSK3β than COS cells, the activity of endogenous GSK3β is assumed higher in PC-3 cells (FIG. 15). In fact, LiCl enhances basal activity of AR transactivation in PC-3 cells in a dose dependent manner (FIG. 16D). LiCl can also enhance AR tranactivation in the presence of androgen in PC-3 cells. The distinct effects of LiCl on AR transactivation in PC-3 vs. COS-1 correlates well with the endogenous GSK3 activity in these two cell lines, indicating that endogenous GSK3β can contribute to the suppression of AR transactivation.

(3) Inhibition of AR Transactivation and PSA Expression by GSK3-3 in LNCaP Cells.

To examine whether the inhibitory effect of GSK3β on AR transactivation extends to cells that express endogenous AR, LNCaP cells which have mutated yet functional AR were cotransfected with the androgen-responsive reporter MMTV-Luc, and GSK3β. As shown in FIG. 17A, addition of GSK3β reduced the activity of AR in a dose-dependent manner. Moreover, addition of LiCl abrogated the GSK3β-mediated inhibition of AR activity. Similar suppression effect also occurred when MMTV-Luc reporter was replaced with ARE4-Luc reporter system.

Prostate-specific antigen (PSA) is a clinically significant androgen-stimulated gene that is used to monitor response to treatment, prognosis, and progression of prostate cancer. Endogenous PSA protein expression was induced by the treatment of LNCaP cells with DHT. This DHT-mediated induction of transcription from the PSA promoter by DHT was repressed by overexpression of wild type GSK3β (FIG. 17B). The results from Northern blot assays further demonstrated that the expression of PSA mRNA was reduced by the ectopic expression of GSK3β (FIG. 17C). Together, both reporter assay and Northern blot assay indicate that GSK3β inhibits AR transactivation and influences expression of the target gene downstream of the AR.

(4) GSK-3B phosphorylates the amino terminus of AR in vitro and inhibits the function of the ligand-independent activation domain (AF-1).

Since the data indicated that GSKβ kinase activity is necessary for inhibiting AR transactivation, the task of determining whether AR is a substrate for GSK3, was undertaken. Three proteins, GST-ARN, GST-AR-DL, and 6His-AR-LBD, that cover most of the N-terminus (aa 38-560), DNA binding and ligand-binding domains (DBD-LBD, aa 551-918), and ligand binding domain (LBD, aa 666-918) of AR, respectively were purified. FIG. 18A demonstrates that GSK3β significantly phosphorylated the GST-AR38-560 (lane 2) while GST protein alone could not be phosphorylated (lane 1). In contrast, under the same experimental conditions GSK3β slightly phosphorylated GST-AR-DBD-LBD (lane 3) or 6His-AR-LBD (lane 4). Thus, it appears that the N-terminus of AR serves as a substrate for GSK3β in vitro.

As AF-1 is located in the N-terminal of AR and FIG. 18A shows GSK3β can phosphorylate AR at the N-terminus, the potential effect of GSK3β on AF-1 function was examined. COS-1 cells were transfected with a fusion construct linking the GAL4 DNA-binding domain to the N-terminal of AR (GAL4-AR-N). The transcriptional response of this construct was assessed using a UAS-Luc reporter (pG5-Luc). FIG. 18B (lower panel) shows that the addition of wild type GSK3β inhibited the constitutive transcriptional activity of GAL4-ARN. In contrast, GSK3β did not influence the activity of GAL4-AR-LBD, which contains the AF-2 domain. These results indicate that GSK3β can suppress AR transactivation via the AF-1 functional domain that is located in the AR N-terminal.

(5) AR Interacts with GSK-3#.

To test whether GSK3β can associate with AR in vitro, the GST pull-down assay was used to examine the interaction between GSK3β and AR. Full length wild type GSK3β was constructed in a GST fusion vector. As shown in FIG. 19A, in vitro translated 35S-methionine-labeled AR was found to bind specifically to purified GST-GSK3β, in the presence or absence of DHT.

To further demonstrate that GSK3β interacts with AR in mammalian cells, co-immunoprecipitation was first used to examine their interaction by cotransfecting AR and HA-tagged-GSK3β into COS-1 cells. The COS-1 cell extracts were immunoprecipitated with an anti-HA antibody. As shown in FIG. 19B, the HA-GSK3 immunocomplexes contained the AR (lane 3), indicating that AR interacts with GSK-3β in the COS-1 cells. HA-tagged GSK3β was also observed in the immunocomplexes pulled down with an anti-AR antibody. Next, LNCaP cells, which express endogenous AR and GSK3β, were used to examine whether GSK3β interacts with AR physiologically. As demonstrated in FIG. 19C, GSK3β forms a stable complex with AR, indicating that GSK3β can interact with AR in the same cell and AR could be a substrate for GSK3β in vivo.

GSK3β suppresses androgen/AR-induced cell growth. As previous reports have revealed that DHT/AR plays important roles in the initiation and progression of prostate cancer, whether the suppression of AR by GSK3β could modulate prostate cancer cell growth was investigated. Inducible S9A-GSK3β plasmids were introduced into the androgen-dependent CWR22R cell line by stable transfection. To distinguish exogenously transfected GSK3β from endogenous GSK3 β in CWR22R cells, a myc-tagged S9A-GSK3β was constructed in the pBIG vector. Doxycycline stimulated the S9A-GSK3β expression in CWR22R-S9A-GSK3β cells but not in the vector transfected CWR22R-pBig cells (FIG. 20A). Using a LUC reporter assay, it was found that induction of S9A-GSK3 reduced AR transactivation by 30% while doxycycline had a marginal effect on CWR22R-pBig cells. This effect likely represents an underestimate of the total impact of GSK3 B on AR activity since CWR22R cells express endogenous GSK3β. To correlate the inhibitory effect of GSK3β on AR with prostrate cancer cell growth, the growth of stable-transfected CWR2R cells was tested in an MTT assay. The MTT assay (FIG. 20C) shows that addition of DHT induced cell growth in both CWR22R-pBig and CWR22R-S9A-GSK3β cells. As expected, the doxycycline treatment caused obvious growth arrest in the CWR22R-S9A-GSK3β cells, but not in the CWR22R-pBig cells. Taken together, these data indicate that activation of GSK3β inhibits AR transcriptional activity and correlates with the reduced cell growth.

(6) Reduction of the Interaction Between AR and ARAs by GSK3β.

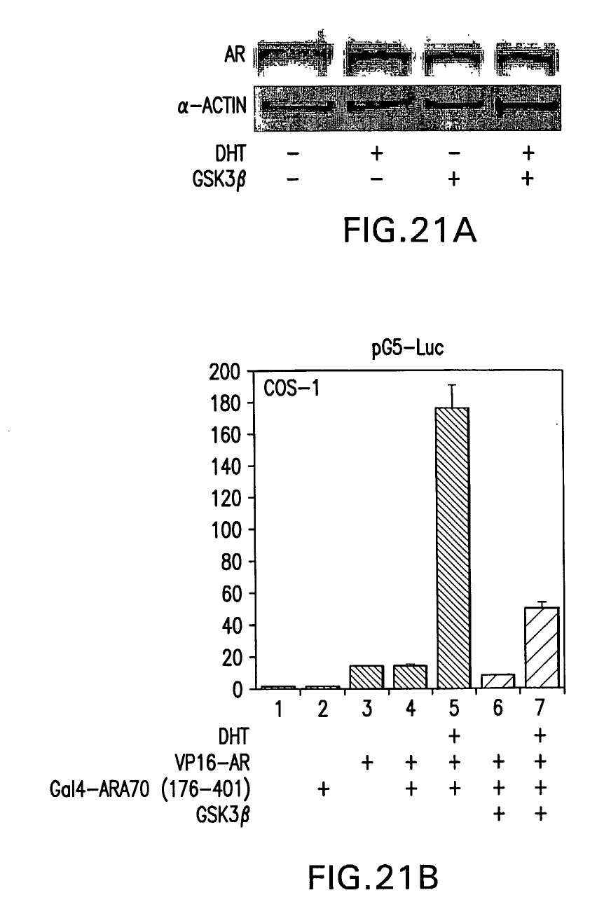

One potential mechanism through which GSK3β can inhibit AR transactivation is by altering the level of AR expression. To address this issue, AR expression was measured by immunoblot in LNCaP cells transfected with the pCMV vector or with pCMV-GSK3β. As shown in FIG. 21A, little change was seen in the endogenous expression of AR in LNCaP cells (FIG. 21A). In addition, AR localization was not altered by expressing S9A-GSK3β LNCaP cells. Similar data were observed in transiently transfected COS-1 and in stably transfected CWR22 cells. These data therefore indicate that GSK3β may not suppress AR transactivation through regulating endogenous androgen receptor stability or distribution.

It is well known that phosphorylation can lead to conformational changes in proteins. Several lines of evidence indicate that kinases may regulate AR activity through modifying the interaction between AR and AR coregulators (Yeh, S., et al. (1999) Proc Natl Acad Sci USA 96 (10), 5458-63, Lin, X. K., et al. (2001) Mol Cell Biol 21 (24), 8385-97, Wang, X., et al. (2002) J Biol Chem 277 (18), 15426-31). A mammalian two-hybrid system was used to study the effects of GSK3β on the interaction between AR and ARA70. GAL4-ARA70 aa176-401 and VP16-AR plasmids were transfected into COS-1 cells. As shown in FIG. 21B, addition of GSK3β inhibited the interaction of AR with ARA70 (lane 7 vs. 5), indicating that the inhibition of AR transactivation by GSK3β can involve reduced interaction between AR and AR coregulators.

A principal clinical problem in prostate cancer treatment is the progression of androgen-dependent tumors to a hormone-refractory state after antiandrogen or androgen ablation therapy. Although the molecular basis for androgen independence is largely unknown, studies of patient specimens indicate that the AR-signaling pathway can be still functional in androgen-refractory cancers. The AR is a phosphorylated protein and its phosphorylation status is associated with its transcriptional activation. The N-terminal of AR contains the majority of the sites phosphorylated in vivo (Kuiper, G. G., et al. (1993) Biochem J291 (Pt 1), 95-101). Alteration of AR phosphorylation by factors with elevated expressions in some prostate cancers is consistent with a mechanism involving phosphorylation stimulating the progression of prostate cancer. These factors include cytokines, growth factors, and G-protein coupled receptors and their activity often leads to the inactivation of GSK30.

Disclosed herein GSK3β modulates AR transcriptional activity by measuring the expression of several androgen-regulated reporters. Specifically, forced overexpression of GSK3β inhibits transcription of PSA in LNCaP prostate cancer cells. Previous studies indicate that protein kinase A (PICA) can activate the AR through modification of its N-terminal domain in the absence of androgen (Sadar, M. D. (1999) JBiol Chem 274 (12), 7777-83). Given that PKA reduces AR phosphorylation (Blok, L. J., et al. (1998) Biochemistry 37 (11), 3850-7), and that the N-terminal of AR mediates the effect of both PKA and GSK3β effect, the results indicate that GSK3β can, in part, regulate the effects of PKA on AR. Future studies are needed to confirm this hypothesis. Since GSK3β is highly active in normal prostate cells, the kinase can inhibit AR transactivation, in the absence or presence of androgen under normal physiological conditions. This hypothesis fits well with the data which shows that the inhibition of GSK3β by LiCl enhances AR activity with or without DHT treatment (FIG. 16). The data demonstrate that GSK3β suppresses AR activity (FIGS. 17, 18) and interacts with AR in vivo (FIG. 19), indicating the AR is a target of GSK3β signaling pathway. Overexpression of constitutively active S9A-GSK3β leads to the growth arrest of prostate cancer cells (FIG. 20), thus, the inhibition of GSK3β can contribute to the development and progression of androgen-independent prostate disease. Considering that PKA, Akt, and MAPK inhibit GSK3β (FIG. 22), the data presented here are consistent with what is known regarding the stimulation of prostate cancer cell growth by growth factors and cytokines, and fit very well with the pro-apoptotic roles of GSK3β in other tissues (Hardt, S. E., et al. (2002) Circ Res 90 (10), 1055-63, Culbert, A. A., et al. (2001) FEBSLett 507 (3), 288-94, Pap, M., et al. (1998) JBiol Chem 273 (32), 19929-32).

Numerous studies have suggested potential links between the androgen/AR and GSK3β signaling pathways. First, testosterone, but not estrogen, prevents the heat shock-induced overactivation of GSK3β, suggesting androgen may display a neuroprotective effect against Alzheimer's disease. Second, GSK3β plays a pivotal role in degradation of the free, cytoplasmic β-catenins, an AR coregulator, through the ubiquitin proteasome pathway. Recent studies indicate that dysregulation of β-catenin expression is found in a variety of human malignancies, including prostate cancer, in which β-catenin may act as a coactivator of AR (Truica, C. I., et al. (2000) Cancer Res 60 (17), 4709-13). Third, GSK3β also phosphorylates c-myc and cyclin D1, resulting in ubiquitin-mediated degradation. This is relevant in that elevated cyclin D1 and c-myc levels may be associated with prostate cancer progression (Chen, Y., et al. (1998) Oncogene 16 (15), 1913-20, Drobnjak, M., et al. (2000) Clin Cancer Res 6 (5), 1891-5, Balaji, K. C., et al. (1997) Urology 51) (6), 1007-15.).

Recent studies also demonstrate that GSK3β may regulate AR activity through β-catenin, an AR coactivator. Disclosed herein GSK3β directly influences AR activity, independent of the β-catenin mediated pathway. The interaction between AR and β-catenin is DHT-dependent, and the data demonstrate that the inhibition of GSK3β by lithium chloride increases AR transcriptional activity in the absence of DHT. Also, several factors that inhibit GSK3β, such as insulin-like growth factor 1 (IGF-1) and insulin, do not stabilize β-catenin or stimulate β-catenin-dependent gene transcription (Ding, V. W., et al. (2000) JBiol Chem 275 (42), 32475-81). This observation argues for the direct effect of GSK3β on AR. Moreover, β-catenin enhances AR activity through interaction with the AR-LBD, which contains the activation function 2 (AF-2) domain. The data indicate that AF-1 activity, but not that of AF-2, is reduced by GSK3β (FIG. 18). Furthermore, GSK3β directly phosphorylates the N-terminal region of AR. The GST-pulldown assay and co-immunoprecipitation assay indicate the interaction between GSK3β and AR (FIG. 19A). Together, these lines of evidence indicate that GSK3β and β-catenin can affect the AR at distinct levels, and that the inhibition of GSK3β followed by elevated β-catenin levels can cooperate to enhance AR activity and lower the requirement for androgen in prostate cancer cells.

AR phospholylation and the resulting inhibition of AR activity contributes to the blockage of DHT-induced cell growth imposed by activated GSK3β (FIG. 20). The phosphorylation of a variety of other substrates by GSK3β can influence cell growth can also be involved. For example, by inhibiting GSK3β, growth factors might promote the dephosphorylation and stabilization of cyclin D1 and c-Myc (Sears, R., et al. (2000) Genes Dev 14 (19), 2501-14, Alt, J. R., et al. (2000) Genes Dev 14 (24), 3102-14, Diehl, J. A., et al. (1998) Genes Dev 12 (22), 3499-511). Elevated cyclin D1 can enhance the activities of cyclin-dependent protein kinases CDK4 and CDK6, resulting in the inactivation of the retinoblastoma gene and entry into the S phase of the cell cycle, c-Myc is known to stimulate prostate cancer cell proliferation and survival, as have been shown in many reports (Kokontis, J., et al. (1994) Cancer Res 54 (6), 1566-73, Miyoshi, Y., et al. (2000) Prostate 43 (3), 225-32). GSK3β is also known to phosphorylate c-Jun, resulting in inhibition of the DNA binding of this transcription factor that has been implicated in cell growth, differentiation, and development (Boyle, W. J., et al. (1991) Cell 64 (3), 573-84, Pfahl, M. (1993) Endocr Rev 14 (5), 651-8). Active GSK3β therefore, is implicated as a key factor in maintenance of the basal states of several important signaling pathways, and dysregulation of GSK3β can lead to transformation to malignancy.

In summary, the data demonstrate that AR is a substrate for GSK3β and that GSK3β negatively regulates AR mediated gene transcription to modulate androgen/AR-mediated cell growth. Molecules which increase the amount of active GSK3β in the cell can be therapeutic molecules and their can be attractive anti androgen receptor activity targets.

### 4. Example 4 Human Checkpoint Protein hRad9 Functions as a Negative Coregulator to Repress Androgen Receptor Transactivation in Prostate Cancer Cells

### a) Materials and Methods

(1) Materials

MMTV-LUC, pCMV-AR, pCDNA3-Flag, pCMX-VP 16-AR have been described previously (Hsu, C. L., et al., J Biol Chem 278:23691-8 (2003), Thin, T. H., et al., J

Biol Chem 278:7699-708 (2003)). pGEX-KG-hRad9 and pCDNA3-AUI-hRad9 were kindly provided by Dr. Larry M. Karnitz, Mayo Clinic, Rochester, M N. (Reconstitution and molecular analysis of the hRad9-hHus1-hRad1 (9-1-1) DNA damage responsive checkpoint complex by Burtelow M A, Roos-Mattjus P M, Rauen M, Babendure J R, and Karnitz L M J B C 276 25903-25909, 2001, which is herein incorporated by reference at least for material related to hRad9). Human multiple tissue Northern (MTN™) Blot II was purchased from BD Biosciences. M2 α-Flag antibody and α-Rad9 antibody (M-389) were purchased from Sigma and Santa Cruz Biotechnology, Inc., respectively.

(2) Yeast Two-Hybrid Screen

The DBD and LBD of AR cDNA was amplified and was cloned into the NdeI and BamHI site of pGBKT7 (Clontech). Yeast strain AH109 was transformed with the vector encoding GAL4 DBD-AR-DBD-LBD fusion and was mated with yeast strain Y187 pretransformed with the human ovary MATCHMAKER cDNA library (Clontech). The yeast clones were selected following the manufacturer's instruction. The positive clones were confirmed by clone lift assay and purified plasmids were retransformed into yeast strain AH109 with bait plasmids. The interaction specificity was further confirmed by liquid 13-galactosidase assay.

(3) Plasmid Constructions

To clone full-length Flag-tagged hRad9, hRad9 cDNA was amplified and cloned into the BamHI and XbaI sites in pCDNA3-Flag vector. Similarly, the cDNA fragments coding aa 1-270 or 269-391 of hRad9 were cloned into pCDNA-Flag to make the expressing vectors for N-terminus of hRad9 and C-terminus of hRad9, respectively. To assemble AR fragments into pGBKT7 vector, fragments covering AR DBD or LBD were inserted with NdeI at the 5′ and BamHI at the 3′ by polymerase chain reaction (PCR) and cloned into the NdeI and BamHI sites in pGBKT7. The QuickChange site-directed mutagenesis kit (Stratagene) was used to mutate the hRad9 sequence. F361 of hRad9 was converted to Ala residue to yield the AXXLF mutant of hRad9 by the Quikchange kit (Stratagene). Similarly, L364 and F365 of hRad9 were converted to Ala residues to yield the FXXAA mutant of bRad9. The mammalian two-hybrid vector of full-length hRad9 was constructed by fusing the hRad9 cDNA in-frame to pCMX-GAL4-DBD. The N-terminus of hRad9 and C-terminus of hRad9 fragments were inserted in-frame to pM vector (Clontech.). DNA vector-Based RNA interference (RNAi) plasmids were used to reduce the endogenous hRad9 expression as previously described (Rahman, M. M., et al., Proc Natl Acad Sci USA 100:5124-9 (2003)). RNAi constructs were designed to target the 56-76, 70-90, 91-110, and 232-252 bp of the hRad9 mRNA sequence relative to the first nucleotide of the start codon and are termed R1, R2, R3, and R4 respectively. The selection of coding sequences was determined empirically and was analyzed by BLAST search to avoid any significant sequence homology with other genes. Vectors that express RNAi under the control of the U6 promoter were constructed by inserting pairs of annealed DNA oligonucleotides into the BS/U6 vector between the ApaI and EcoRI sites. All plasmids were verified by sequencing.

(4) Cell Culture and Transfections

PC-3, CWR22R, and LNCaP cell lines were maintained in RPMI-1600 supplemented with 10% fetal calf serum (FCS). Transient transfection for luciferase assays was carried out in 24-well plates (5×10⁴ cells per well) using SuperFect as described previously (Lin, H. K., et al., Embo J 21:4037-48 (2002)). DNA mixtures in transfection assay were indicated in each figure. The total amount of transfected DNA was kept constant (1 μg) by adding the corresponding amounts of empty expression plasmids. After transfection, cells were cultured in RPMI-1600 supplemented with 5% charcoal-stripped FCS in the presence or absence of 10 nM dihydrotestosterone (DHT) for 18 h. Luciferase assays were performed as previously described (Yang, L., et al., J Biol Chem 278:16820-7 (2003)). In Western blotting assays, CWR22R or LNCaP cells were transfected by electroporation using 5×10⁶ cells/0.4 ml of RPMI medium containing 2% FCS plus 9 μg of the indicated plasmids. 1 μg of EGFP expression vector was used for transfection efficiency. Electroporation was performed at 250 V and 950 μF using Gene Pulser II (Bio-Rad)

(5) In Vitro GST Pull-Down Assays

The N-terminus (N), DBD, LBD, and DBD-LBD of AR were in vitro translated in the presence of [³⁵S] methionine using T7 polymerase and the coupled transcription/translation kit (Promega). pGEX-KG-hRad9 plasmids expressing GST-hRad9 fusion protein were transformed into BL21 (DE3) bacteria strain. 0.4 mM isopropyl-o-D-thiogalactopyranoside was added into LB medium containing transformed bacteria when the OD600 reached 0.5. Bacteria were further cultured in 30° C. for 3 h and lysed by 4 cycles of freezing-thawing in NETN buffer (20 mM Tris/pH 8.0, 0.5% NP-40, 100 mM NaCl, 6 mM MgCl₂, 1 mM EDTA, 1 mM DTT, 8% glycerol, and 1 mM PMSF). The GST-hRad9 fusion proteins were purified with glutathione-beads in 4° C. Labeled proteins of AR mutants were incubated with equal amounts of GST-hRad9 in binding buffer (50 mM Hepes, 100 mM NaCl, 20 mM Tris-Cl/pH 8.0, 0.1% Tween 20, 10% glycerol, 1 mM dithiothreitol, 0.5 mM phenylmethysulfonyl fluoride, 1 mM NaF, and 0.4 mM sodium vanadate) with or without 10 nM DHT at 4° C. for 2 h. The beads were then washed with NETN buffer 4 times, resuspended in SDS-polyacrylamide loading buffer, and resolved on 10% SDS-polyacrylamide gel electrophoresis followed by autoradiograhy.

(6) Co-Immunoprecipitation Assays and Western Blotting

293T cells were transfected in 10-cm dishes with 2.5 μg Flag-hRad9 and 7.5 μg pCMV-AR plasmids in the presence or absence of 10 nM DHT as indicated. Total cell extract was prepared in the presence of absence of 10 nM DHT in immunoprecipitation buffer (50 mM Tris-HCl/pH 8.0, 150 mM NaCl, 20% glycerol, 0.5% NP-40, 50 mM NaF, mM NaF, 0.4 mM sodium vanadate, 0.5 mM phenylmethysulfonyl fluoride, and 0.5 mM DTT). After centrifugation, supernatants were incubated for 2 h with M2 α-Flag antibody or normal anti-mouse serum. For CWR22R cells, cell extracts were prepared as above and supernatants were precipitated by ct-AR antibody (554225, BD Biosciences) or normal anti-mouse serum. Precipitated protein complexes were washed 4 times either in the presence or absence of 10 nM DHT and subsequently analyzed by Western blotting.

(7) Real-Time PCR

Prostate cancer specimens were collected at the time of radical prostatectomy, representing specimens from clinical prostate cancers. All histological diagnoses were confirmed by staining parallel sections with H&E. Total RNA was isolated using the Trizol (Gibco) reagent, according to the manufacturer's instructions, and 1 μg RNA was subjected to reverse transcription using Superscript II (Invitrogen, CA). Specific primers for hRAD9,5′-CGCTGTAAGATCCTGATGAAGTC-3′ (forward) (SEQ ID NO:17) and 5′-tgcctcctcctcgtggtag-3′ (reverse) (SEQ ID NO: 18), were designed according to Bacon Designer2 software. 18s rRNA primers, 5′-tgccttccttggatgtggtag-3′ (forward) (SEQ ID NO: 19), and 5′-cgtctgccctatcaactttcg-3′ (reverse) (SEQ ID NO: 20), were used as controls. The real-time PCR was performed with 1 μl RT product, 12.5 μl 2XSYBR Green PCR Master Mix (Biorad), and 0.5 μl of each primer (10 μM), in a total volume of 25 μl. PCR was performed with 94° C. for 3 min, and 40 cycles of 94° C. for 15 s, 60° C. for 30 s, and 72° C. for 30 s on an iCycler iQ Multi-color real-time PCR detection system (Bio-Rad). Each sample was run in triplicate. Data were analyzed by iCycler iQ software (Bio-Rad).

### b) Results

(1) Ligand-dependent Interaction of AR and hRad9 in Yeast

In order to screen proteins with ligand-dependent interaction with AR, the human AR DBD-LBD was fused with the DBD of GAL4 functioned as bait in yeast two-hybrid screening (FIG. 23A). A pretransformed normal human ovary cDNA library was screened in the presence of 10 nM DHT. A total of 1×10⁸ individual yeast clones were first selected by nutrition deprivation and confirmed to activate P-gal by clone-lift assay. Sequence analyses showed three clones, which encoded aa 327-391 of hRad9 in-frame with the GAL4 activation domain. The hRad9 fragment from yeast lies in the C-terminus of hRad9 and contains an FXXLF (aa.361-365) motif that overlaps with the potential nuclear localization sequence (NLS) motif (aa.356-364) (Hirai, I., and H. G. Wang, J Biol Chem 277:25722-7 (2002)). This fragment of hRad9 is referred to as f-hRad9 (FIG. 23B). Liquid β-gal assay was performed to quantitatively analyze the interaction between AR and hRad9. Constructs containing either f-hRad9 peptide (a.a.327-391) or ARA70, an AR coactivator, showed a strong induction with the AR-DBD-LBD in the presence of DHT (FIG. 23C). As a negative control, GAL4 activation domain alone was not able to interact with AR. Thus, these results indicate an androgen-dependent interaction between AR and hRad9 in yeast.

(2) Analysis of hRad9 Expression

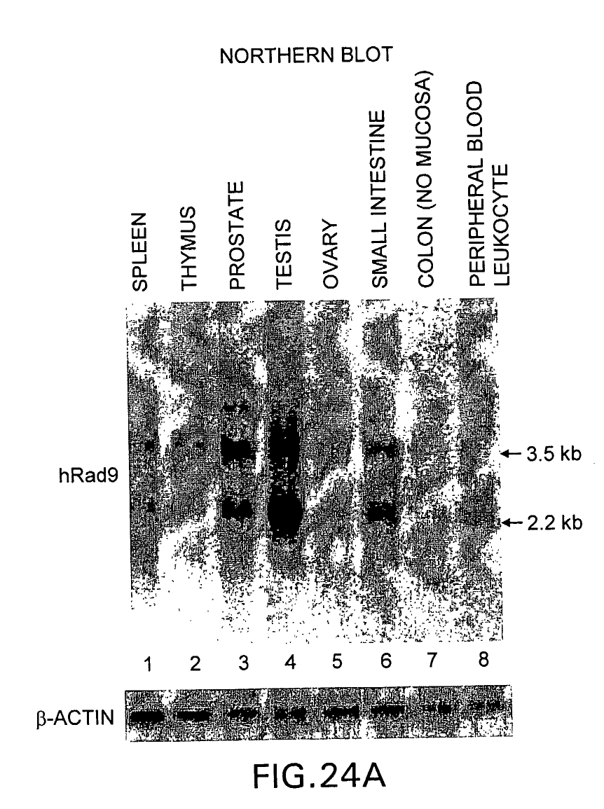

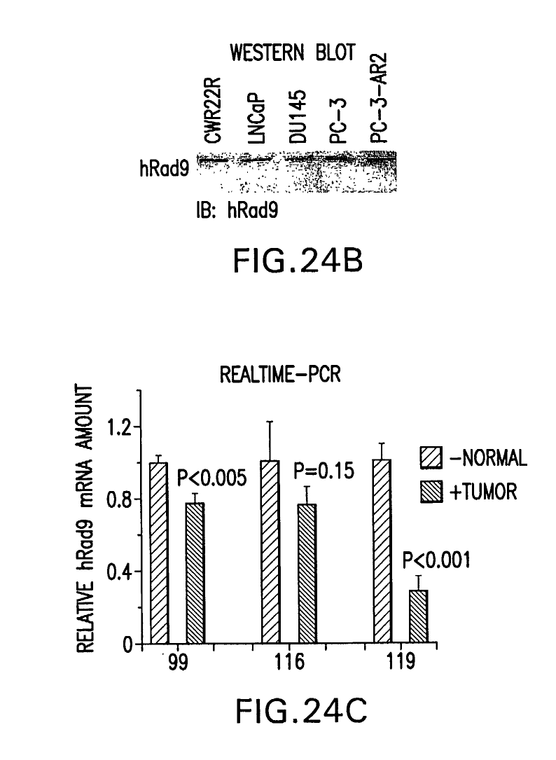

Northern hybridization analyses were carried out to determine the expression of hRad9 in various human tissues, especially the reproductive organs. Since the hRad9 N-terminus is homologous with PCNA, a specific probe was used covering the last 121 amino acid residues of hRad9 proteins. As shown in FIG. 24A, hRad9 was ubiquitously expressed at variable levels in all eight tissues examined. When normalized to β-actin mRNA levels, hRad9 mRNA was found at the highest levels in testis, second highest in prostate, and the lowest level in colon. Interestingly, hHus1 mRNA was found to be most abundant in testis where hRad1 also expressed at high levels (Hang, H., and H. B. Lieberman, Genomics 65:24-33 (2000)). It is tempting to speculate that hRad9 may likely contribute to the meiotic checkpoint in testis where the maintenance of genomic DNA integrity is extremely important.

The prostate is made up of epithelial glands and a fibromuscular stroma with prostate cancers arising from the glandular epithelium (Feldman, B. J., and D. Feldman, Nat Rev Cancer 1:34-45 (2001)). To determine hRad9 expression in prostate cancers, immunoblot analyses of variable prostate cancer cell lysates were performed, revealing an anti-hRad9-reactive band in all cells examined (FIG. 24B). In agreement with previous reports (Greer, D. A., et al., p. 4829-35, Cancer Res, vol. 63 (2003), Hirai, I., and H. G. Wang, J Biol Chem 277:25722-7 (2002)), fluorescence analyses using GFP-hRad9 fusion proteins indicated hRad9 protein was localized mainly in the nucleus. Since AR also translocates into nucleus upon androgen treatment, hRad9 and AR proteins can be colocalized in the nucleus.

The expression of hRad9 in human prostate samples under normal or pathologic situations using quantitative real-time PCR were also analyzed. All three samples were obtained from patients with high-grade prostatic adenocarcinoma. Compared to the adjacent normal area, it was found that the neoplastic tissues express significantly less amounts of hRad9 as revealed by real-time-PCR analyses (FIG. 24C) in some patients that were examined. Although this result is intriguing, more samples may need to be analyzed before it can be established whether hRad9 expression is frequently down-regulated in advanced prostate cancers.

(3) hRad9 Associates with AR In Vivo

To determine whether hRad9 and AR interact in mammalian cells, the f-hRad9 fragment was subcloned into the mammalian pM expression vector. Mammalian two-hybrid assays were carried out in PC-3 cells in the absence and presence of 10 nM DHT. As shown in FIG. 25A, androgen-dependent interactions were detected between GAL4-f-hRad9 and fall length AR (lane 2). The interaction between AR and the C-terminus of ARA54 was used as a positive control (FIG. 25A, lane 3). Furthermore, the C-terminus of hRad9 (aa 269-391) displayed a strong interaction with AR in the presence of androgen while the PCNA-like domain of hRad9 (N-hRad9, aa 1-270) did not (FIG. 25A, lane 5 and 4, respectively), indicating the C-terminus of hRad9 mediates the interaction with AR.

To further investigate the physical association of full-length hRad9 (FL-hRad9) with AR, mammalian two-hybrid assays were performed with FL-hRad9 fused to the DBD of GAL4 and full length AR fused to VP16. As seen in FIG. 25B, androgen stimulated the interaction between full length AR and hRad9 while hydroxyflutamide (HF), an antagonist for AR, inhibited the androgen-induced interaction between AR and hRadg. Furthermore, 293T cells were cotransfected with AR and Flag epitope-tagged hRad9 to test whether AR existed in hRad9 immunoprecipitates. An AR band was detected in the Flag-hRad9 immunoprecipitates (FIG. 25C). Finally, coimmunoprecipitation of native proteins from a prostate cancer cell line CWR22R extract confirmed the AR-hRad9 association in vivo (FIG. 25D). Together, the association between AR and hRad9 is unequivocal in mammalian cells.

(4) Domains of AR Involved in Binding to hRad9

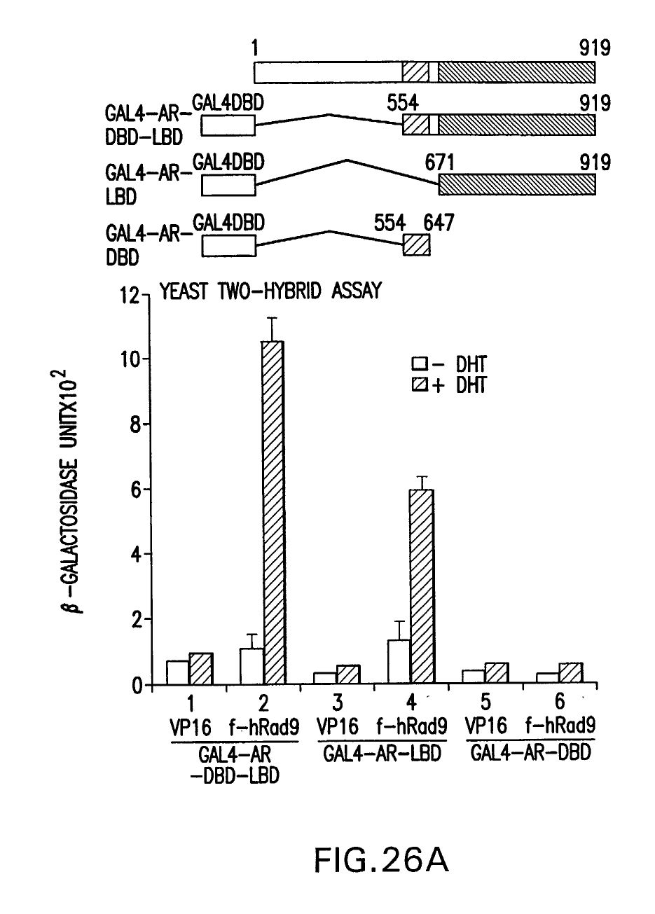

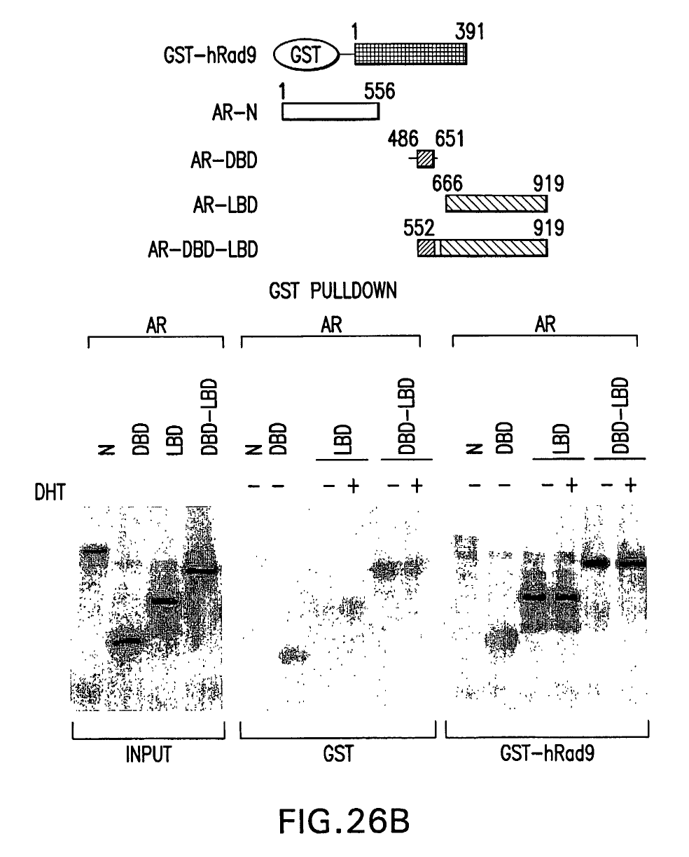

While the C-terminus of hRad9 associates with AR, it was of interest to determine which domain (s) of AR is responsible for the interaction. Yeast two-hybrid assays were performed first in AH109 yeast cells, different regions of AR fused with GAL4 DBD were cotransformed with the plasmid containing VP 16 activation domain (VP16-AD) or VP 16-AD fused with amino acids 327-391 of hRad9 (VP16-f-Rad9) in the presence or absence of 10 nM DHT. In the absence of androgen, there was little interaction between VP16-hRad9 and various GAL4-AR fusion proteins (FIG. 26A, open bars). However, with the treatment of 10 nM DHT (FIG. 26A, closed bars), coexpression of VP16-f-hRad9 and GAL4-AR-DBD-LBD yielded an increased reporter activity by 10-fold over that with GAL4-AR-DBD-LBD and VP16 AD (FIG. 26A, lane 2 vs. lane 1). As expected, VP16-f-hRad9 also interacted with AR LBD in the presence of androgen (FIG. 26A, lane 4). Though GAL4-AR-DBD did not interact with hRad9 (FIG. 26A, lane 6), the interaction between hRad9 and AR LBD was weaker than the association between AR DBD-LBD and hRad9, indicating DBD domain might also contribute to the proper folding of AR-DBD-LBD in yeast.

Since two-hybrid assays provide an indirect measurement of protein interactions, to investigate whether Rad9 interacts directly with AR LBD, GST pull-down assays were performed using GST protein alone or GST-Rad9 fusion protein. The various domains of AR were labeled with [³⁵S]-methionine by in vitro translation and incubated with GST-hRad9-bound beads. As shown in FIG. 26B, the AR LBD and the AR-DBD-LBD interacted specifically with Rad9. Unlike the interaction observed in the yeast two-hybrid system or the mammalian two-hybrid system, the presence or absence of androgen did not robustly influence the interaction between AR and hRad9. Consistent with previous studies, this discrepancy of ligand-dependent manner can be because the high concentration of proteins in GST pull-down assays can reduce the binding sensitivity between AR and its be associated with many other proteins that interrupt the AR-hRad9 association in the absence of ligand. Neither the N-terminus of AR (aa. 1-556) nor the DBD alone adhered to GST-hRad9. Therefore, these results are consistent with the yeast two-hybrid experiments and indicate that the AR LBD is required for the interaction with Rad9.

(5) FXXLF Motif Mediates AR-hRad9 Interaction

The LXXLL motif was first identified in some steroid receptor coactivators (Heery, D. M., et al., Nature 387:733-6 (1997)). However, among steroid receptors, AR appears to be relatively unique as it interacts with only a very limited subset of LXXLL sequences (Chang, C. Y., and D. P. McDonnell., Mol Endocrinol 16:647-60 (2002)). Previous studies showed that the FXXLF motif plays important roles in mediating the interaction of the AR LBD with several FXXLF-containing AR coregulators (He, B., et al., J Biol Chem 275:22986-94 (2000); He, B., et al., J Biol Chem 277:10226-35 (2002)). Interestingly, one FXXLF motif is located at the carboxyl-terminus of hRad9 (aa 361-365). To investigate whether this FXXLF motif contributes to the association between AR and hRad9, mutants of hRad9 at this FXXLF motif were tested with mammalian two-hybrid assays. Mutations of the FXXLF motif in Rad9 decreased dramatically the interaction between AR and the fragment of hRad9 (aa 327-391), shown by either the AXXLF or FXXAA mutants (FIG. 27A, lane 3, 4 vs. lane 2, closed bars). Similarly, AXXLF or FXXAA mutants reduced the interaction between AR and full-length hRad9 (FIG. 27B, lane 3, 4 vs. lane 2, closed bars), indicating this FXXLF motif is critical for hRad9 to interact with AR.

However, LXXLL or FXXLF motifs fail to predict precisely the interaction between AR and these motifs. For example, FXXLF motif peptides derived from the CBP (FGSLF) and p300 (FGSLF) fail to interact with AR (He, B., et al., J Biol Chem 277:10226-35 (2002)). Moreover, the mutants of FXXLF motif in hRad9 might eliminate the AR-hRad9 interaction because of the whole conformation change of hRadg, not limited to the FXXLF m-helix. Thus, it was of interest to determine whether the FXXLF motif in hRad9 can directly interact with AR. Therefore, a small peptide containing the FXXLF motif of hRad9 was fused with GAL4-DBD (FIG. 27C upper panel), cotransfected with VP16-AR, and tested in the absence and presence of 10 nM DHT in two-hybrid peptide assays. Androgen-dependent interactions were demonstrated between VP16-AR and the GAL4-FXXLF fusion peptides (FIG. 27C). As a positive control, a 350 fold DHT-dependent interaction with a GAL4-D30 peptide was observed, which contains a LXXLL motif that interacts with AR as described previously (Chang, C. Y., and D. P. McDonnell., Mol Endocrinol 16:647-60 (2002)). Together, the data demonstrate that the FXXLF motif in C-terminus of hRad9 mediates the interaction with the AR.

(6) hRad9 Specifically Represses AR-mediated Transactivation

To understand the consequence of hRad9 binding to the AR, AR transactivation was studied with the MMTV-LUC reporter in PC-3 cells. The promoter of MMTV-LUC is a naturally occurring MMTV-long terminal repeat (LTR) which contains androgen-responsive elements (ARE). Cotransfection of wild type hRad9 with AR decreased the transcriptional activity of AR in a dose-dependent manner (FIG. 28A, lanes 3-5), whereas FXXAA mutants had only marginal effect on AR transactivation (FIG. 28A, lanes 6-8). Neither wild type (WT) nor FXXAA mutant of hRad9 had an effect on the transcriptional activity in the absence of 10 nM DHT, indicating that they do not affect the basal transcriptional activity. Similar results were observed when PC-3 cells were replaced with LNCaP cells.

To determine the effect of endogenous hRad9 on AR, CWR22R cells were transfected with several siRNA constructs targeting hRad9 (R1, R2, R3, and R4) or mock-transfected as control. After 2 days of transfection, the protein levels of hRad9 were evaluated by immunoblot analyses with anti-hRad9 antibodies. Whereas R2 and R4 siRNA constructs only marginally reduced endogenous hRad9 expression and R3 moderately decreased hRad9 expression (FIG. 28B, lanes 3, 5 and 4), R1 siRNA dramatically reduced the hRad9 protein in CWR22R cells (FIG. 28B lane 2). Therefore, the influence of siRNA R1 on AR transcriptional activity was tested in CWR22R cells. R1 siRNA increased the DHT-induced activation of the MMTV-LUC reporter in a dose-dependent manner (FIG. 28C), indicating the repressive effect of endogenous hRad9 on AR. Similar results were observed when CWR2R cells were replaced with PC-3 cells.

Prostate-specific antigen (PSA) is a clinically significant androgen-stimulated gene that is used to monitor response to treatment and progression of prostate, cancer (Debes, J. D., and D. J. Tindall, Cancer Lett 187:1-7 (2002)). Endogenous PSA protein expression was induced by the DHT treatment in LNCaP cells (FIG. 28D, lane 2). Addition of hRad9 potently inhibited the DHT-mediated induction of PSA (FIG. 28D, lane 4). Taken together, these data showed, for the first time, an involvement of hRad9 in AR transcriptional activation.

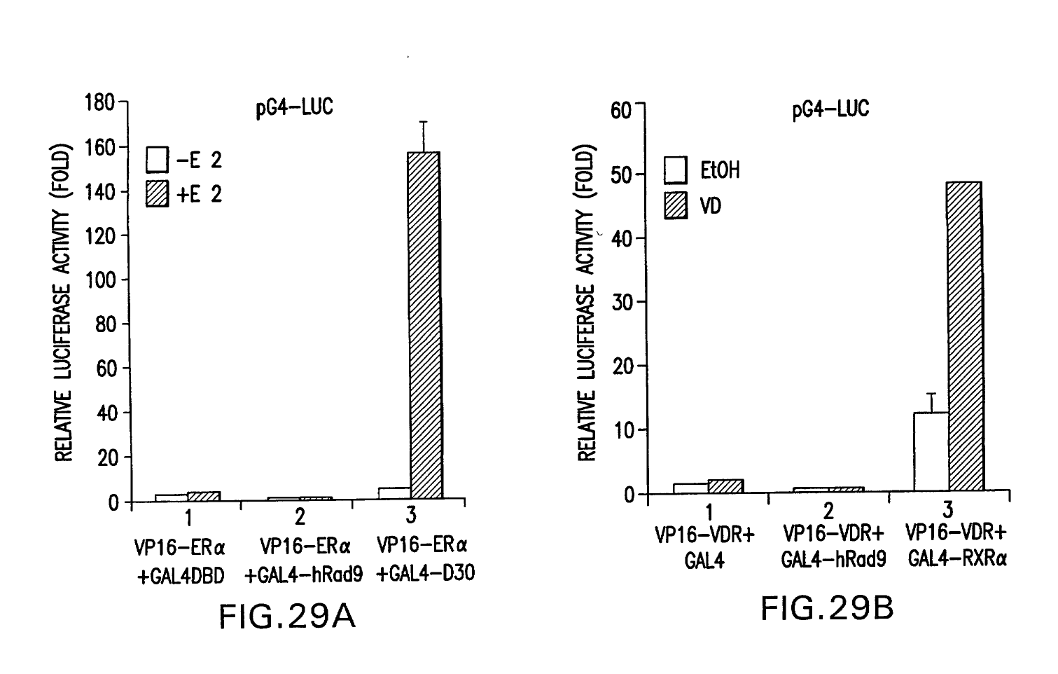

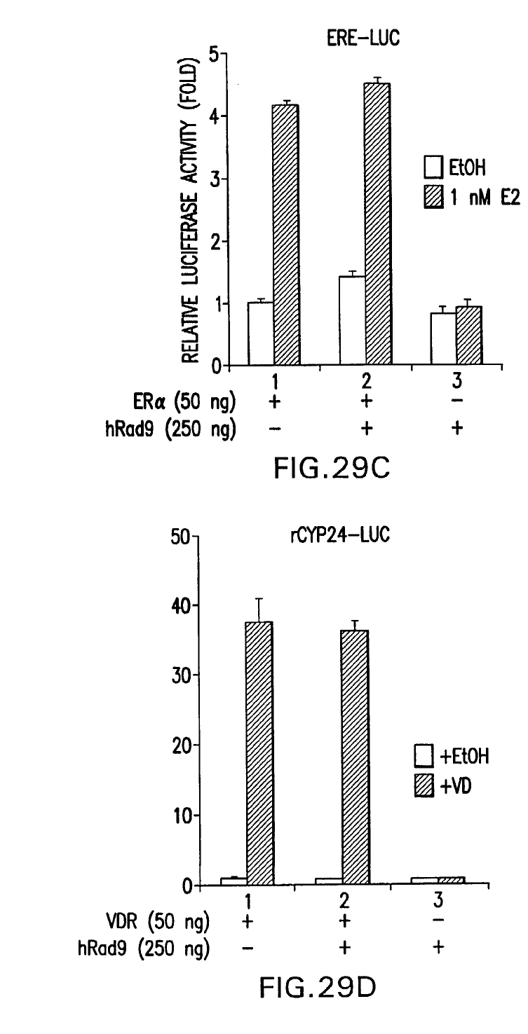

To determine whether hRad9 can interact with other steroid receptors and further affect their transactivation, the possible association of hRad9 with the estrogen receptor α (ERα) or the vitamin D receptor (VDR) in mammalian two-hybrid system was examined. In the presence of estrogen, ERcc showed strong interaction with GAL4D30 (FIG. 29A, lane 3), whereas there was no interaction with hRad9 (FIG. 29A, lane 2). Similarly, VDR associated with GAL4-RXRα (FIG. 29B, lane 3), however, there was no interaction of hRad9 with VDR (FIG. 29B, lane 2). As previous studies reported FXXLF is a motif specific for AR coregulators (He, B., et al., J Biol Chem 277:10226-35 (2002)), it is not surprising that hRad9 is more specific to AR as compared to other steroid receptors since the studies showed that the FXXLF motif in hRad9 mediates the interaction between hRad9 and AR. ERE-LUC and rCyp24-LUC reporter plasmids were used to demonstrate the transcriptional activity of ERa and VDR, respectively. As shown in FIGS. 29C and 29D, whereas the ER and VDR could induce luciferase activity in the presence of their cognate ligands in PC-3 cells, cotransfection of hRad9 had little inhibitory effect on their transcriptional activity.

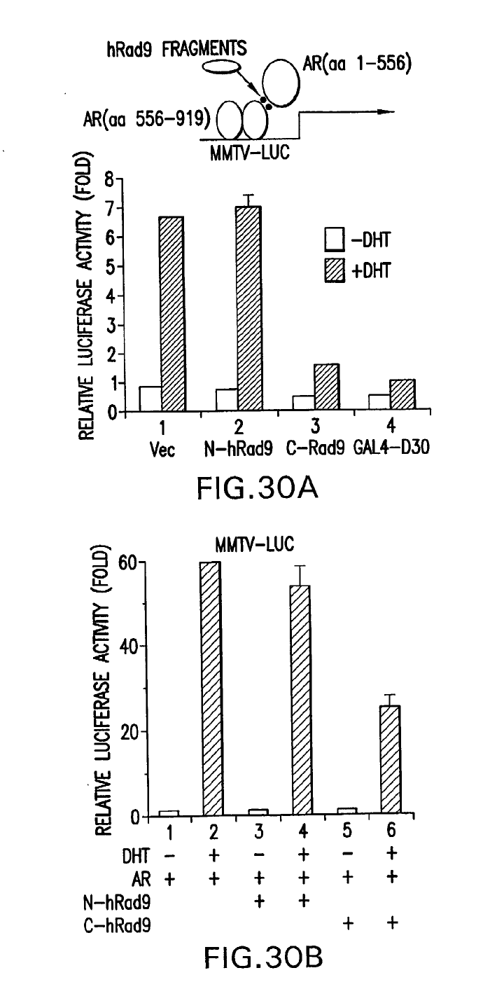

hRad9 Suppressed the AR N/C interaction—Early reports suggested the FXXLF motif in AR N-terminus is important for interacting with the C-terminus of AR and this interaction is required for fall capacity of AR transactivation (Hsu, C. L., et al., J Biol Chem 278:23691-8 (2003)). While the C-terminus of hRad9 contains the FXXLF motif and interacts with the LBD, it is possible that Rad9 can influence the AR N/C interaction. As previously described (Chang, C. Y., and D. P. McDonnell., Mol Endocrinol 16:647-60 (2002)), a reconstituted AR transcription assay was used to address this possibility (FIG. 30A, upper panel). In PC-3 cells, the AR DBD-LBD (aa. 556-919) displayed minimal transactivation even in the presence of DHT, consistent with previous studies showing AR LBD only has minimal transcriptional activity. However, coexpression of the N-terminus of AR (aa 1-556) with AR DBD-LBD restores agonist-induced transactivation (FIG. 30A, lower panel, lane 1). The GAL4-D30 was used as a positive control in this experiment to show the blockage of the N/C interaction in AR (FIG. 30A, lane 4). The C-terminus of Rad9 can potently inhibit the interaction between AR N- and C-terminus in the presence of androgen (FIG. 30A, lane 3), whereas the N-terminus of hRad9, which cannot interact with AR, has no effect on N—C interaction (FIG. 30A, lane 2). Furthermore, the full length AR was applied to test whether the C-terminus of hRad9 can block intact AR transactivation. The data demonstrated only the C-terminus of hRad9, not the N-terminus of Rad9, suppressed AR-mediated transactivation (FIG. 30B). Together, these results indicate one mechanism by which disruptions of AR N/C interaction by the hRad9 FXXLF motif might contribute to the inhibitory role of Rad9 on AR. Consequently, the binding between other coactivators and AR can be blocked due to the lack of a stabilized N/C interaction which is necessary for AR activation.

Studies in Schizosaccharomnyces pombe and human cells have demonstrated a conserved checkpoint pathway, including hRad9, hHus1, and hRad1, capable of causing cell cycle arrest in response to incomplete DNA replication or DNA damage (al-Khodairy, F., et al., Mol Biol Cell 5:147-60 (1994), Freire, R., et al., Genes Dev 12:2560-73 (1998), Kaur, R., et al., Mol Biol Cell 12:3744-58 (2001), Kostrub, C. F., et al., Embo J 17:2055-66 (1998)). Later studies demonstrated that hRad9, hHus1 and hRad1 form a stable heterotrimeric complex, called the 9-1-1 complex, with a clamp structure similar to PCNA (Thelen, M. P., et al., Cell 96:769-70 (1999)). Biochemical, biophysical, and molecular modeling studies suggest that Rad17 may help load the 9-1-1 complex onto sites of DNA damage in the checkpoint signaling pathway (Rauen, M., et al., J Biol Chem 275:29767-71 (2000)). Since DNA damage induces hRad17-dependent association of 9-1-1 with chromatin, it is believed that the 9-1-1 complex is involved in the direct recognition of DNA lesions and initiates the checkpoint responses (Zou, L., et al., Genes Dev 16:198-208 (2002)). Nonetheless, the hRad9 C-terminal region is not involved in the interaction with hRad1 or hHus1, and is exposed outside of the 9-1-1 clamp structure (Roos-Mattjus, P., et al., J Biol Chem 278:24428-37 (2003)). This flexible structure of hRad9 C-terminus leads to the possibility that it can play important roles in interacting with other proteins and subsequently regulate other signal transduction pathways. Indeed, the C-terminal region of hRad9 (aa. 270-391) contains a predicted NLS (aa. 356-364) which can act to guide the 9-1-1 complex into the nucleus (Hirai, I., and H. G. Wang, J Biol Chem 277:25722-7 (2002)). The SH3 domain of c-Ab1 also interacts directly with the C-terminal region of hRad9 (64), hRad9 interacts with replication and checkpoint protein topoisomerase II beta binding protein 1 through the C-terminal 17 amino acids of hRad9 (Greer, D. A., et al., Cancer Res 63:4829-35 (2003)). Furthermore, several phosphorylation sites were identified in the hRad9 C-terminal region that can play critical roles in the transduction of downstream checkpoint signals (Roos-Mattjus, P., et al., J Biol Chem 278:24428-37 (2003), St. Onge, R. P., et al., J Biol Chem 278:26620-8 (2003)). Present studies add a new role for the hRad9 C-terminus, to modulation of AR transcriptional activity through its interaction with the AR LBD via its FXXLF motif (FIG. 27), which links directly a key player in DNA damage detection and repair with AR-mediated transcription in prostate cancer.

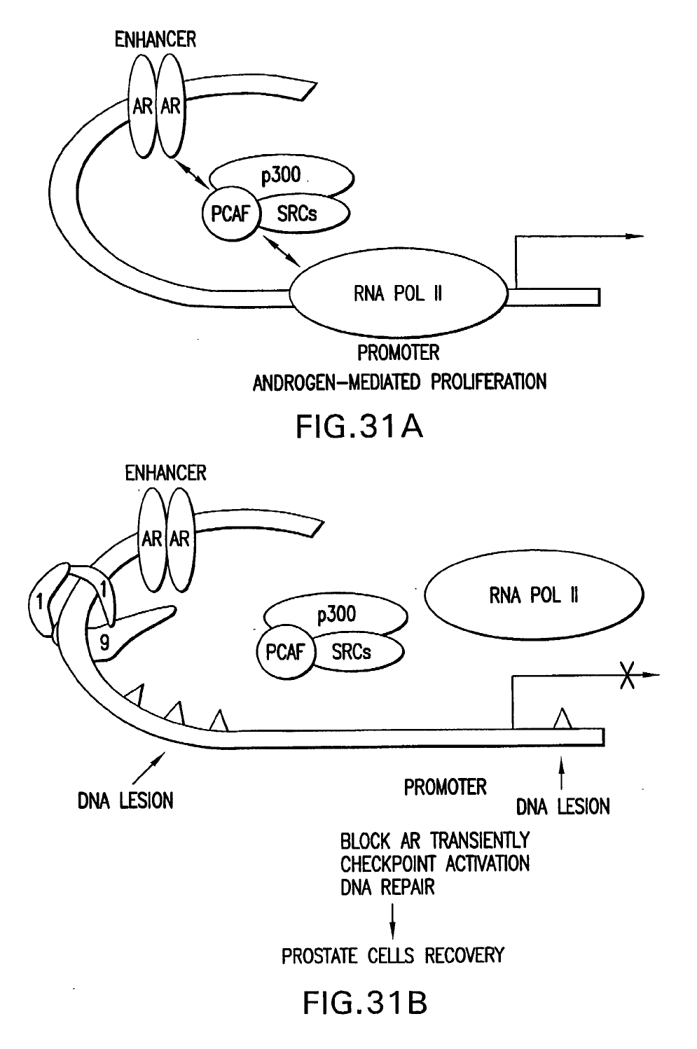

Clinical studies have shown that androgen ablation improved the survival of patients with locally advanced prostate cancer when combined with radiation therapy (Bolla, M., D. et al., N Engl J Med 337:295-300 (1997)). Furthermore, the use of animal models have suggested androgen can protect prostate cancer from apoptosis induced by radiotherapy (Joon, D. L., et al., Int J Radiat Oncol Biol Phys 38:1071-7 (1997)). Studies using prostate cancer cell lines also demonstrate that androgen plays protective roles in LNCaP cells exposed to radiation or chemotherapeutic agents (Berchem, G. J., et al., Cancer Res 55:735-8 (1995), Coffey, R. N., et al., Prostate 53:300-9 (2002)). However, the mechanism underling the protective effect of androgen remains largely unknown. The findings that hRad9 functions as a corepressor for AR can open up several avenues of investigation. Though prostate cancer has a low proliferative index, it is noteworthy that prostate cancer cells show high rates of mutation, indicating DNA lesions can occur frequently in prostate cancer cells (Hara, T., J. et al., Cancer Res 63:149-53 (2003)). With evidence showing that hRad9 functions as a negative regulator of the AR-mediated transcription (FIG. 28), a possible mechanism was provided for prostate cancer cells to reduce the potential cell proliferation at the moment when cells are repairing the DNA lesions. Loss of hRad9 in cells can decrease the cell ability to repair DNA lesions and increase cell proliferation mediated by androgen/AR (FIG. 31). Interestingly, the preliminary analyses using a few prostate cancer samples show the expression of hRad9 is reduced in prostate tumors as compared to normal prostatic tissue (FIG. 24C). This fits the above hypothesis and indicates that dysregulated expression of hRad9 can be involved in the progression of prostate cancer. Early studies also showed hRad9 may play roles in the modulation of cell cycle progrogression (Siede, W., et al., Proc Natl Acad Sci USA 90:7985-9 (1993)). Blocking of hRad9 expression showed reduced ionizing radiation-induced accumulation of G2-M cells and more cells bypassed the G2 checkpoint after ionizing radiation or hydroxyurea treatment (Hirai, I., and H. G. Wang, J Biol Chem 277:25722-7 (2002)). Furthermore, previous reports demonstrate that hRad9, as well as hHus1 might act as tumor suppressors through their functions of maintaining chromosome integrity (Cai, R. L., Y. et al., J Biol Chem 275:27909-16 (2000)). Therefore, these two functions of hRad9, repressing AR activity and DNA damage checkpoint, could interdependently prevent cell transformation in prostate cancer development.

Finally, the data (FIG. 30) demonstrated that hRad9 can suppress AR transcriptional activity via interrupting the AR N/C interaction. Previous studies suggested that AR N/C interaction might play essential roles for AR transcriptional activity. Several AR coactivators, such as SRC-1 and CBP, were shown to be able to promote AR N/C interaction (McInerney, E. M., et al., Proc Natl Acad Sci USA 93:10069-73 (1996)). Conversely, SMRT and Filamin-A, two AR corepressors, were shown to inhibit AR activity through disruption of the AR N/C interaction and/or competition with the p160 coactivators (Liao, G., et al., J Biol Chem 278:5052-61 (2003), Ngan, E. S., et al., 22:734-9 (2003)). However, whether these coregulators may utilize their LXXLL or FXXLF motifs to affect AR N/C interaction is not clear. The reasons that the FXXLF motif in hRad9 strongly interacts with the AR LBD may be: 1) two positive amino acid residues (K³⁵⁹ and K³⁶⁰) lie at the N-terminus of FXXLF; 2) no positively charge amino acid residues are located near the C-terminus of FXXLF; 3) there are no amino acid residues, such as glycine and proline, which can interrupt the FXXLF α-helix structure in FXXLF. Thus, hRad9 fits quite well in the model recently proposed for FXXLF motif binding to AR LBD (He, B., and E. M. Wilson, Mol Cell Biol 23:2135-50 (2003)).

In summary, hRad9 was identified as a corepressor of AR. hRad9 interacts with AR LBD through its C-terminus and reduces AR transcriptional activity by interrupting the AR N/C interaction. Further studies may help to better understand the connection between hRad9 and AR in prostate cancers.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### G. REFERENCES

379. Aberle, H., Bauer, A., Stappert, J., Kispert, A., and Kemler, R. (1997) Embo J 16 (13), 3797-804.
380. al-Khodairy, F., E. Fotou, K. S. Sheldrick, D. J. Griffiths, A. R. Lehman, and A. M. Cart. 1994. Identification and characterization of new elements involved in checkpoint and feedback controls in fission yeast. Mol Biol Cell 5:147-60.
381. Alt, J. R., Cleveland, J. L., Hannink, M., and Diehl, J. A. (2000) Genes Dev 14 (24), 3102-14.
382. Amundadottir, L. T. and P. Leder. 1998. Signal transduction pathways activated and required for mammary carcinogenesis in response to specific oncogenes. Oncogene 16: 737-46.
383. Balaji, K. C., Koul, H., Mitra, S., Maramag, C., Reddy, P., Menon, M., Malhotra, R. K., and Laxmanan, S. (1997) Urology 51) (6), 1007-15.
384. Bayer-Garner, I. B. and B. Smoller. 2000. Androgen receptors: a marker to increase sensitivity for identifying breast cancer in skin metastasis of unknown primary site. Mod Pathol 13:119-22.
385. Beals, C. R., Sheridan, C. M., Turck, C. W., Gardner, P., and Crabtree, G. R. (1997) Science 275 (5308), 1930-4.
386. Beitel, L. K., L. Prior, D. M. Vasiliou, B. Gottlieb, M. Kaufman, R. Lumbroso, C. Alvarado, B. McGillivray, M. Trifiro, and L. Pinsky. 1994. Complete androgen insensitivity due to mutations in the probable alpha-helical segments of the DNA-binding domain in the human androgen receptor. Hum Mol Genet 3:21-7.
387. Berchem, G. J., M. Bosseler, L. Y. Sugars, H. J. Voeller, S. Zeitlin, and E. P. Gehmann. 1995. Androgens induce resistance to bcl-2-mediated apoptosis in LNCaP prostate cancer cells. Cancer Res 55:735-8.
388. Berrino, F., P. Muti, A. Micheli, G. Bolelli, V. Krogh, R. Sciajno, P. Pisani, S. Panico, and G. Secreto. 1996. Serum sex hormone levels after menopause and subsequent breast cancer. J Natl Cancer Inst 88: 291-6.
389. Bessho, T., and A. Sancar. 2000. Human DNA damage checkpoint protein hRAD9 is a 3′ to 5′ exonuclease. J Biol Chem 275:7451-4.
390. Bieche, I., B. Parfait, S. Tozlu, R. Lidereau, and M. Vidaud. 2001. Quantitation of androgen receptor gene expression in sporadic breast tumors by real-time RT-PCR: evidence that MYC is an AR-regulated gene. Carcinogenesis 22:1521-6.
391. Birrell, S. N., J. M. Bentel, T. E. Hickey, C. Ricciardelli, M. A. Weger, D. J. Horsfall, and W. D. Tilley. 1995. Androgens induce divergent proliferative responses in human breast cancer cell lines. J Steroid Biochem Mol Biol 52: 459-67.
392. Bledsoe, R. K., V. G. Montana, T. B. Stanley, C. J. Delves, C. J. Apolito, D. D. McKee, T. G. Consler, D. J. Parks, E. L. Stewart, T. M. Willson, M. H. Lambert, J. T. Moore, K. H. Pearce, and H. E. Xu. 2002. Crystal structure of the glucocorticoid receptor ligand binding domain reveals a novel mode of receptor dimerization and coactivator recognition. Cell 110: 93-105.
393. Blok, L. J., de Ruiter, P. E., and Brinkmann, A. O. (1998) Biochemistry 37 (11), 3850-7.
394. Bocchinfuso, W. P., W. P. Hively, J. F. Couse, H. E. Varmus, and K. S. Korach. 1999. A mouse mammary tumor virus-Wnt-1 transgene induces mammary gland hyperplasia and tumorigenesis in mice lacking estrogen receptor-alpha. Cancer Res 59: 1869-76.
395. Bolla, M., D. Gonzalez, P. Warde, J. B. Dubois, R. O. Mirimanoff, G. Storme, J. Bernier, A. Kuten, C. Sternberg, T. Gil, L. Collette, and M. Pierart. 1997. Improved survival in patients with locally advanced prostate cancer treated with radiotherapy and goserelin. N Engl J Med 337:295-300.
396. Bonnette, S. G. and D. L. Hadsell. 2001. Targeted disruption of the IGF-I receptor gene decreases cellular proliferation in mammary terminal end buds. Endocrinology 142: 4937-45.
397. Boyle, W. J., Smeal, T., Defize, L. H., Angel, P., Woodgett, J. R., Karin, M., and Hunter, T. (1991) Cell 64 (3), 573-84.
398. Brisken, C., A. Ayyannan, C. Nguyen, A. Heineman, F. Reinhardt, J. Tan, S. K. Dey, G. P. Dotto, R. A. Weinberg, and T. January 2002. IGF-2 is a mediator of prolactin-induced morphogenesis in the breast. Dev Cell 3: 877-87.
399. Brown, T. R. 1995. Human androgen insensitivity syndrome. J Androl 16:299-303.
400. Brys, M., M. Wojcik, H. Romanowicz-Makowska, and W. M. Krajewska. 2002. Androgen receptor status in female breast cancer: RT-PCR and Western blot studies. J Cancer Res Clin Oncol 128: 85-90.
401. Burfeind, P., Chemicky, C. L., Rininsland, F., and Ilan, J. (1996) Proc Natl Acad Sci U S A 93 (14), 7263-8.
402. Burn, K., Duggan, B., Atlinson, E. A., Famulslki, K. S., Nemer, M., Bleackley, R. C., and Michalak, M. 1994. Modulation of gene expression by calreticulin binding to the glucocorticoid receptor. Nature 367:476-480.
403. Cai, R. L., Y. Yan-Neale, M. A. Cueto, I-I. Xu, and D. Cohen. 2000. HDAC1, a histone deacetylase, forms a complex with Hus 1 and Rad9, two G2/M checkpoint Rad proteins. Biol Chem 275:27909-16.
404. Chang, C. Y., and D. P. McDonnell. 2002. Evaluation of ligand-dependentchanges in AR structure using peptide probes. Mol Endocrinol 16:647-60.
405. Chang, C., A. Saltzman, S. Yeh, W. Young, E. Keller, H. J. Lee, C. Wang, and A. Mizokami. 1995. Androgen receptor: an overview. Crit Rev Eukaryot Gene Expr 5:97-125.
406. Chang, C., and Kokontis, J. 1988. Identification of a new member of the steroid receptor super-family by cloning and sequence analysis. Biochem. Biophys. Res. Commun. 155:971-977.
407. Chang, C., da Silva, S. L., Ideta, R., Lee, Y., Yeh, S., and Burbach, J. P. H. 1994. Human and rat TR4 orphan receptors specify a subclass of the steroid receptor superfamily. Proc. Natl. Acad. Sci. USA 91:6040-6044.
408. Chang, C., J. D. Norris, H. Gron, L. A. Paige, P. T. Hamilton, D. J. Kenan, D. Fowlkes, and D. P. McDonnell. 1999. Dissection of the LXXLL nuclear receptor-coactivator interaction motif using combinatorial peptide libraries: discovery of peptide antagonists of estrogen receptors alpha and beta. Mol Cell Biol 19:8226-39.
409. Chang, C. S., J. Kokontis, and S. T. Liao. 1988. Molecular cloning of human and rat complementary DNA encoding androgen receptors. Science 240: 324-6.
410. Chen, J. D., and Evans, R. M. 1995. A transcriptional co-repressor that interacts with nuclear hormone receptors. Nature 377:454-457.
411. Chen, Y., Martinez, L. A., LaCava, M., Coghlan, L., and Conti, C. J. (1998) Oncogene 16 (15), 1913-20.
412. Chesire, D. R., Ewing, C. M., Gage, W. R., and Isaacs, W. B. (2002) Oncogene 21 (17), 2679-94.
413. Coffey, R. N., R. W. Watson, A. J. O'Neill, K. Mc Eleny, and J. M. Fitzpatrick. 2002. Androgen-mediated resistance to apoptosis. Prostate 53:300-9.
414. Cohen, P., and Frame, S. (2001) Nat Rev Mol Cell Biol 2 (10), 769-76.
415. Cohen, P., Nimmo, H. G., and Proud, C. G. (1978) Biochem Soe Symp 43, 69-95
416. Couse, J. F. and K. S. Korach. 1999. Estrogen receptor null mice: what have we learned and where will they lead us? Endocr Rev 211: 358-417.
417. Cross, D. A., Alessi, D. R., Cohen, P., Andjekovich, M., and Hemmings, B. A. (1995) Nature 378 (6559), 785-9.
418. Culbert, A. A., Brown, M. J., Frame, S., Hagen, T., Cross, D. A., Bax, B., and Reith, A. D. (2001) FEBSLett 507 (3), 288-94.
419. Culig, Z., Hobisch, A., Cronauer, M. V., Radmayr, C., Trapman, J., Hittmair, A., Bartsch, G., and Klocker, H. (1994) Cancer Res 54 (20), 5474-8.
420. Culig, Z., Hobisch, A., Cronauer, M. V., Radrnayr, C., Trapman, J., Hittmair, A., Bartsch, G., and Klocker, H. (1995) Eur Urol 27 (Suppl 2), 45-7.
421. Darimont, B. D., R. L. Wagner, J. W. Apriletti, M. R. Stallcup, P. J. Kushner, J. D. Baxter, R. J. Fletterick, and K. R. Yamamoto. 1998. Structure and specificity of nuclear receptor-coactivator interactions. Genes Dev 12:3343-56.
422. de Groot, R. P., Auwerx, J., Bourouis, M., and Sassone-Corsi, P. (1993) Oncogene 8 (4), 841-7.
423. Debes, J. D., and D. J. Tindall. 2002. The role of androgens and the androgen receptor in prostate cancer. Cancer Lett 187:1-7.
424. Dedhar, S., PSR, Shago, M., Hagesteijn, C. L., Yang, H., Filmus, J., Hawley, R. G., Bruchovsky, N., Cheng, H., Matusik, R. J., and Giguere, V. 1994. Inhibition of nuclear hormone receptor activity by calreticulin. Nature 367:480-483.
425. Di Monaco, M., E. Brignardello, L. Leonardi, V. Gatto, and G. Boccuzzi. 1995. Inhibitory effect of hydroxyflutamide plus tamoxifen on oestradiol-induced growth of MCF-7 breast cancer cells. J Cancer Res Clin Oncol 121:710-4.
426. Diehl, J. A., Cheng, M., Roussel, M. F., and Sherr, C. J. (1998) Genes Dev 12 (22), 3499-511.
427. Dimitrakakis, C., J. Zhou, and C. A. Bondy. 2002. Androgens and mammary growth and neoplasia. Fertil Steril 77 Suppl 4: 26-33.
428. Ding, V. W., Chen, R. H., and McCormick, F. (2000) JBiol Chem 275 (42), 32475-81.
429. Dotzlaw, H., Moehren, U., Mink, S., Cato, A. C., Iniguez Lluhi, J. A., and Baniahmad, A. 2002. The amino terminus of the human AR is target for corepressor action and antihormone agonism. Mol. Endocrinol. 16:661-673.
430. Drobnjak, M., Osman, I., Scher, H. I., Fazzari, M., and Cordon-Cardo, C. (2000) Clin Cancer Res 6 (5), 1891-5.
431. Feldman, B. J., and D. Feldman. 2001. The development of androgen-independent prostate cancer. Nat Rev Cancer 1:34-45.
432. Feng, W., R. C. Ribeiro, R. L. Wagner, H. Nguyen, J. W. Apriletti, R. J. Fletterick, J. D. Baxter, P. J. Kushner, and B. L. West. 1998. Hormone-dependent coactivator binding to a hydrophobic cleft on nuclear receptors. Science 280:1747-9.
433. Forman, B. M. and Samuels, H. H. 1990. Interactions among a subfamily of nuclear hormone receptors: the regulatory zipper model. Mol. Endocrinol. 4:1293-1301.
434. Freire, R., J. R. Mnrguia, M. Tarsounas, N. F. Lowndes, P. B. Moens, and S. P. Jackson. 1998. Human and mouse homologs of Schizosaccharomyces pombe rad 1 (+) and Saccharomyces cerevisiae RAD 17: linkage to checkpoint control and mammalian meiosis. Genes Dev 12:2560-73.
435. Fu, M., Wang, C., Reutens, A. T., Wang, J., Angeletti, R. H., Siconolfi-Baez, L., Ogryzko, V., Avantaggiati, M., L., and Pestell, R. G. 2000. p300 and p300/cAMP-response element-binding protein-associated factor acetylate the androgen receptor at sites governing hormone-dependent transactivation. J. Biol. Chem. 275:20853-20860.
436. Fujimoto, N., Yeh, S., Kang, H., Inui, S., Chang, H., Mizokami, A., and Chang, C. 1999. Cloning and characterization of androgen receptor coactivator, ARA55, in human prostate. J. Biol. Chem. 274:8316-8321.
437. Galigniana, M. D., Hoousley, P. R., DeFranco, D. B., and Pratt, W. B. 1999. Inhibition of glucocorticoid receptor nucleocytoplasmic shuttling by okadaic acid requires intact cytoskeleton. J. Biol. Chem. 275:16222-16227.
438. Galigniana, M. D., Scruggs, J. L., Herrington, J., Welsh, M. J., Carter-Su, C., Housley, P. R., and Pratt, W. B. 1998. Heat shock protein 90-dependent (geldanamycin-inhibited) movement of the glucocorticoid receptor through the cytoplasm to the nucleus requires intact cytoskeleton. Mol. Endocrinol. 12:1903-1913.
439. Gao, Y., and Pimplikar, S. W. 2001. The γ-secretase-cleaved C-terminal fragment of amyloid precursor protein mediates signaling to the nucleus. Proc. Natl. Acad. Sci. USA 98:14979-14984.
440. Gerdes, M. J., Dang, T. D., Larsen, M., and Rowley, D. R. 1998. Transforming growth factor-131 induces nuclear to cytoplasmic distribution of androgen receptor and inhibits androgen response in prostate smooth muscle cells. Endocrinology 139:3569-3577.
441. Gittes, R. F. (1991) NEnglJMed324 (4), 236-45.
442. Glass, C. K., and M. G. Rosenfeld. 2000. The coregulator exchange in transcriptional functions of nuclear receptors. Genes Dev 14:121-41.
443. Greene, G. L. 2003. In vivo imaging reveals estrogen receptor's hidden personality. Nat Med 9: 22-3.
444. Greenlee, R. T., Murray, T., Bolden, S., and Wingo, P. A. (2000) CA Cancer J Clin 50 (1), 7-33.
445. Greer, D. A., B. D. Besley, K. B. Kennedy, and S. Davey. 2003. hRad9 rapidly binds DNA containing double-strand breaks and is required for damage-dependent topoisomerase II beta binding protein 1 focus formation. Cancer Res 63:4829-35.
446. Hagmann, M. 1999. Checkpoint gene linked to human cancer. Science 286:2433-4.
447. Hall, J. L., Chatham, J. C., Eldar-Finkelman, H., and Gibbons, G. H. (2001) Diabetes 50 (5), 1171-9.
448. Hammond, J., Le, Q., Goodyer, C., Gelfand, M., Trifiro, M., and LeBlanc, A. 2001. Testosteron-mediated neuroprotection through the androgen receptor in human primary neurons. J. Neruochem. 77:1319-1326.
449. Hang, H., and H. B. Lieberman. 2000. Physical interactions among human checkpoint control proteins HUS 1p, RAD 1p, and RAD9p, and implications for the regulation of cell cycle progression. Genomics 65:24-33.
450. Hara, T., J. 3/liyazaki, It. Araki, M. Yamaoka, N. Kanzaki, M. Kusaka, and M. Miyamoto. 2003. Novel mutations of androgen receptor: a possible mechanism of bicalutamide withdrawal syndrome. Cancer Res 63:149-53.
451. Hardt, S. E., and Sadoshima, J. (2002) Circ Res 90(10), 1055-63.
452. Hartwell, L. H., and M. B. Kastan. 1994. Cell cycle control and cancer. Science 266:1821-8.
453. Harwood, A. J., Plyte, S. E., Woodgett, J., St-mR, H., and Kay, R. R. (1995) Cell 80 (1), 139-48.
454. Haslam, S. Z. 1988. Acquisition of estrogen-dependent progesterone receptors by normal mouse mammary gland. Ontogeny of mammary progesterone receptors. J Steroid Biochem 31: 9-13.
455. He, B., and E. M. Wilson. 2003. Electrostatic modulation in steroid receptor recruitment of LXXLL and FXXLF motifs. Mol Cell Biol 23:2135-50.
456. He, B., J. A. Kemppainen, and E. M. Wilson. 2000. FXXLF and WXXLF sequences mediate the NH2-terminal interaction with the ligand binding domain of the androgen receptor. J Biol Chem 275:22986-94.
457. He, B., J. T. Minges, L. W. Lee, and E. M. Wilson. 2002. The FXXLF motif mediates androgen receptor-specific interactions with coregulators. J Biol Chem 277:10226-35.
458. He, B., Kemppainen, J. A., and Wilson, E. M. 2000. FALF and WALF sequences mediate the NH₂-terminal interaction with the ligand binding domain of the androgen receptor. J. Biol. Chem. 275:22986-22994.
459. He, B., Kemppainen, J. A., Voegel, J. J., Gronemeyer, H., and Wilson, E. M. 1999. Activation function 2 in the human androgen receptor ligand binding domain mediates interdomain communication with the NH₂-terminal domain. J. Biol. Chem. 274:37219-37225.
460. He, B., Meng, Y. H., and Mivechi, N. F. (1998) Mol Cell Bio 18(11), 6624-33.
461. Heery, D. M., E. Kallhoven, S. Hoare, and M. G. Parker. 1997. A signature motif in transcriptional co-activators mediates binding to nuclear receptors. Nature 387:733-6.
462. Heinlein, C. A., and Chang, C. 2002. Androgen receptor (AR) coregulators: an overview. Endocr. Rev. 23:175-200.
463. Hermanson, O., C. K. Glass, and M. G. Rosenfeld. 2002. Nuclear receptor coregulators: multiple modes of modification. Trends Endocrinol Metab 13: 55-60.
464. Hewitt, S. C., W. P. Bocchinfuso, J. Zhai, C. Harrell, L. Koonce, J. Clark, P. Myers, and K. S. Korach. 2002. Lack of ductal development in the absence of functional estrogen receptor alpha delays mammary tumor formation induced by transgenic expression of ErbB2/neu. Cancer Res 62: 2798-805.
465. Hirai, I., and H. G. Wang. 2002. A role of the C-terminal region of human Rad9 (hRad9) in nuclear transport of the hRad9 checkpoint complex. J Biol Chem 277:25722-7.
466. Hoeflich, K. P., Luo, J., Rubie, E. A., Tsao, M. S., Jin, O., and Woodgett, J. R. (2000) Nature 406 (6791), 86-90.
467. Honma, N., G. Sakamoto, F. Akiyama, Y. Esaki, M. Sawabe, T. Arai, T. Hosoi, N. Harada, M. Younes, and K. Takubo. 2003. Breast carcinoma in women over the age of 85: distinct histological pattern and androgen, oestrogen, and progesterone receptor status. Histopathology 42: 120-7.
468. Horlein, A. J., Naar, A. M., Heinzel, T., Torchia, J., Gloss B., Kurokawa, R., Ryan, A., Kamei, Y., Soderstrom, M., and Glass, C. K. 1995. Ligand-independent repression by the thyroid hormone receptor mediated by a nuclear receptor co-repressor. Nature 377:397-404
469. Hsiao, P., and Chang, C. 1999. Isolation and characterization of ARA160 as the first androgen receptor N-terminal-associated coactivator in human prostate cells. J. Biol. Chem. 274:22373-22379.
470. Hsiao, P., Lin, D., Nakao, R., and Chang, C. 1999. The linkage of Kennedy's neuron disease to ARA24, the first identified androgen receptor polyglutamine region-associated coactivator. J. Biol. Chem. 274:20229-20234.
471. Hsu, C. L., Y. L. Chen, S. Yeh, H. J. Ting, Y. C. Hu, H. Lin, X. Wang, and C. Chang. 2003. The use of phage display technique for the isolation of androgen receptor interacting peptides with (F/W)XXL (F/W) and FXXLY new signature motifs. J Biol Chem 278:23691-8.
472. Ikonen, T., Palvimo, J. J., and Janne O. A. 1997. Interaction between the amino and carboxyl terminal regions of rat androgen receptor modulates transcriptional activity and is influenced by nuclear receptor coactivator. J. Biol. Chem. 272:29821-29828.
473. Inoue, S., A. Orimo, T. Hosoi, S. Kondo, H. Toyoshima, T. Kondo, A. Ikegami, Y. Ouchi, H. Orimo, and M. Muramatsu. 1993. Genomic binding-site cloning reveals an estrogen-responsive gene that encodes a RING finger protein. Proc Natl Acad Sci USA 90: 11117-21.
474. Jepsen, K., O. Hermanson, T. M. Onami, A. S. Gleiberman, V. Lunyak, R. J. McEvilly, R. Kurokawa, V. Kumar, F. Liu, E. Seto, S. M. Hedriek, G. Mandel, C. K. Glass, D. W. Rose, and M. G. Rosenfeld. 2000. Combinatorial roles of the nuclear receptor corepressor in transcription and development. Cell 102:753-63.
475. Jiang, J. G., A. Bell, Y. Liu, and R. Zarnegar. 1997. Transcriptional regulation of the hepatocyte growth factor gene by the nuclear receptors chicken ovalbumin upstream promoter transcription factor and estrogen receptor. J Biol Chem 272: 3928-34.
476. Joon, D. L., M. Hasegawa, C. Sikes, V. S. Khoo, N. H. Terry, G. K. Zagars, M. L. Meistrich, and A. Pollack. 1997. Supraadditive apoptotic response of R3327-G rat prostate tumors to androgen ablation and radiation. Int J Radiat Oncol Biol Phys 38:1071-7.
477. Kang, H. Y., H. K. Lin, Y. C. Hu, S. Yeh, K. E. Huang, and C. Chang. 2001. From transforming growth factor-beta signaling to androgen action: identification of Smad3 as an androgen receptor coregulator in prostate cancer cells. Proc Natl Acad Sci USA 98:3018-23.
478. Kang, H. Y., S. Yeh, N. Fujimoto, and C. Chang. 1999. Cloning and characterization of human prostate coactivator ARA54, a novel protein that associates with the androgen receptor. J Biol Chem 274:8570-6.
479. Kato, S., H. Endoh, Y. Masuhiro, T. Kitamoto, S. Uchiyama, H. Sasaki, S. Masushige, Y. Gotoh, E. Nishida, H. Kawashima, and et al. 1995. Activation of the estrogen receptor through phosphorylation by mitogen-activated protein kinase. Science 270: 1491-4.
480. Kaur, R., C. F. Kostrub, and T. Enoch. 2001. Structure-function analysis of fission yeast Hus 1-Rad1-Rad9 checkpoint complex. Mol Biol Cell 12:3744-58.
481. Kleinberg, D. L., M. Feldman, and W. Ruan. 2000. IGF-I: an essential factor in terminal end bud formation and ductal morphogenesis. J Mammary Gland Biol Neoplasia 5: 7-17.
482. Knudsen, K. E., Cavenee, W. K., and Arden, K. C. 1999. D-type cyclins complex with the androgen receptor and inhibit its transcriptional transactivation ability. Cancer Res. 59:2297-2301.
483. Koklontis, J., Takakura, K., Hay, N., and Liao, S. (1994) Cancer Res 54(6), 1566-73.
484. Komatsu, K., T. Miyashita, H. Hang, K. M. Hopkins, W. Zheng, S. Cuddeback, M. Yamada, H. B. Lieberman, and H. G. Wang. 2000. Human homologue of S. pombe Rad9 interacts with BCL-2/BCL-xL and promotes apoptosis. Nat Cell Biol 2:1-6.
485. Kostrub, C. F., K. Knudsen, S. Subramani, and T. Enoch. 1998. Huslp, a conserved fission yeast checkpoint protein, interacts with Radlp and is phosphorylated in response to DNA damage. Embo J 17:2055-66.
486. Krylova, O., Messenger, M. J., and Salinas, P. C. (2000) J Cell Biol 151(1), 83-94.
487. Kuenen-Boumeester, V., T. H. Van der Kwast, W. L. van Putten, C. Claassen, B. van Ooijen, and S. C. Henzen-Logrnans. 1992. Immunohistochemical determination of androgen receptors in relation to oestrogen and progesterone receptors in female breast cancer. Int J Cancer 52:581-4.
488. Kuiper, G. G., de Ruiter, P. E., Trapman, J., Boersma, W. J., Grootegoed, J. A., and Brinkmann, A. O. (1993) Biochem J291 (Pt 1), 95-101.
489. Langley, E., Z. X. Zhou, and E. M. Wilson. 1995. Evidence for an anti-parallel orientation of the ligand-activated human androgen receptor dimer. J Biol Chem 270:29983-90.
490. Lau, K. F., Miller, C. C., Anderton, B. H., and Shaw, P. C. (1999) JPept Res 54 (1), 85-91.
491. Laudet, V., Hänni, C., Coll, J., Catzeflis, F. and Stéhelin, D. 1992. Evolution of the nuclear receptor gene superfamily. EMBO J. 11:1003-1013.
492. Lavinsky, R. M., Jeppsen, K., Heinzel, T., Torchia, J., Mullen, T., schiff, R., Del-Rio, A., L., Ricote, M., Ngo, S., Gemsch, J., Hilfenbech, S. G., Osborne, C. K., Glass, C. K., Rosenfeld, M. G., and Rose, D. W. 1998. Diverse signaling pathways modulate nuclear receptor recruitment of N-CoR and SMRT complex. Proc. Natl. Acad. Sci. USA 95:2920-2925.
493. Lea, O. A., S. Kvinnsland, and T. Thorsen. 1989. Improved measurement of androgen receptors in human breast cancer. Cancer Res 49:7162-7.
494. Lee, D. K., Duan, H. O., and Chang, C. 2000. From androgen receptor to the general transcription factor TFIIH: indication of cdk activating kinase (CAK) as an androgen receptor NH2-terminal associated coactivator. J. Biol. Chem. 275:9308-9313.
495. Lee, L. F., Guan, J., Qiu, Y., and Kung, H. J. (2001) Mol Cell Biol 21 (24), 8385-97.
496. Liao, G., Chen, L. Y., Zhang, A., Godavarthy, A., Xia, F., Ghosh, J. C., Li, H., and Chen, J. D. 2003. Regulation of androgen receptor activity by the nuclear receptor corepressor SMRT. J. Biol. Chem. 278:5052-5061.
497. Lieberman, H. B., K. M. Hopkins, M. Nass, D. Demetrick, and S. Davey. 1996. A human homolog of the Schizosaccharomyces pombe Rad9+ checkpoint control gene. Proc Natl Acad Sci USA 93:13890-5.
498. Lin, H. K., L. Wang, Y. C. Hu, S. Altuwaijri, and C. Chang. 2002. Phosphorylation-dependent ubiquitylation and degradation of androgen receptor by Akt require Mdm2 E3 ligase. Embo J 21:4037-48.
499. Lin, H. K., Yeh, S., Kang, H. Y., and Chang, C. (2001) Proc Natl Acad Sci USA 98 (13), 7200-5.
500. Llopis, J., S. Westin, M. Ricote, Z. Wang, C. Y. Cho, R. Kurokawa, T. M. Mullen, D. W. Rose, M. G. Rosenfeld, R. Y. Tsien, C. K. Glass, and J. Wang. 2000. Ligand-dependent interactions of coactivators steroid receptor coactivator-1 and peroxisome proliferator-activated receptor binding protein with nuclear hormone receptors can be imaged in live cells and are required for transcription. Proc Natl Acad Sci USA 97:4363-8.
501. Lobaccaro, J. M., S. Lumbroso, C. Belon, F. Galtier-Dereure, J. Bringer, T. Lesimple, M. Namer, B. F. Cutuli, H. Pujol, and C. Sultan. 1993. Androgen receptor gene mutation in male breast cancer. Hum Mol Genet. 2:1799-802.
502. Lydon, J. P., F. J. DeMayo, C. R. Funk, S. K. Mani, A. R. Hughes, C. A. Montgomery, Jr., G. Shyamala, O. M. Conneely, and B. W. O'Malley. 1995. Mice lacking progesterone receptor exhibit pleiotropic reproductive abnormalities. Genes Dev 9: 2266-78.
503. Mangelsdorf, D. J., C. Thummel, M. Beato, P. Herrlich, G. Schutz, K. Umesono, B. Blumberg, P. Kastner, M. Mark, P. Chambon, and et al. 1995. The nuclear receptor superfamily: the second decade. Cell 83:835-9.
504. Markus, S. M., Taneja, S. S., Logan, S. K., Li, W., Ha, S., Hittelman, A. B., Rogatsky, I., and Garabedian, M. J. 2002. Identification and characterization of ART-27, a novel coactivator for the androgen receptor N terminus. Mol. Biol. Cell 13:670-682.
505. Maucher, A. and E. von Angerer. 1993. Antiproliferative activity of casodex (ICI 176.334) in hormone-dependent tumours. J. Cancer. Res Clin Oncol 119: 669-74.
506. McInerney, E. M., D. W. Rose, S. E. Flynn, S. Westin, T. M. Mullen, A. Krones, J. Inostroza, J. Torchia, R. T. Nolte, N. Assa-Munt, M. V. Milburn, C. K. Glass, and M. G. Rosenfeid, 1998. Determinants of coactivator LXXLL motif specificity in nuclear receptor transcriptional activation. Genes Dev 12:3357-68.
507. McInerney, E. M., M. S. Tsai, B. W. O'Malley, and B. S. Katzenellenbogen. 1996. Analysis of estrogen receptor transcriptional enhancement by a nuclear hormone receptor coactivator. Proc Natl Acad Sci USA 93:10069-73.
508. McKenna, N. J., and B. W. O'Malley. 2002. Minireview: nuclear receptor coactivators—an update. Endocrinology 143:2461-5.
509. McKenna, N. J. and B. W. O'Malley. 2002. Combinatorial control of gene expression by nuclear receptors and coregulators. Cell 1118: 465-74.
510. McKenna, N. J., Lanz, R. B., and O'Malley, B. W. 1999. Nuclear Receptor Coregulators: Cellular and Molecular Biology. Endocr. Rev. 20:321-344.
511. Meyer, M. E., Gronemeyer, H., Turcotte, B., Bocquel, M. T., Tasset, D., and Chambon, P. 1989. Steroid hormone receptors compete for factors that mediate their enhancer function. Cell 57:433-442.
512. Migliaccio, A., G. Castoria, M. Di Domenico, A. de Falco, A. Bilancio, M. Lombardi, M. V. Barone, D. Ametrano, M. S. Zannini, C. Abbondanza, and F. Auricchio. 2000. Steroid-induced androgen receptor-oestradiol receptor beta-Src complex triggers prostate cancer cell proliferation. Embo J 19: 5406-17.
513. Miyoshi, Y., Uemura, H., Fujinami, K., Mikata, K., Harada, M., Kitamura, H., Koizumi, Y., and Kubota, Y. (2000) Prostate 43 (3), 225-32.
514. Ngan, E. S., Y. Hashimoto, Z. Q. Ma, M. J. Tsai, and S. Y. Tsai. 2003. Overexpression of Cdc25B, an androgen receptor coactivator, in prostate cancer. Oncogene 22:734-9.
515. Niemann, C., V. Brinkmann, E. Spitzer, G. Hartmann, M. Sachs, H. Naundorf, and W. Birchmeier. 1998. Reconstitution of mammary gland development in vitro: requirement of c-met and c-erbB2 signaling for branching and alveolar morphogenesis. J Cell Biol 143: 533-45.
516. Nyberg, K. A., R. J. Michelson, C. W. Putnam, and T. A. Weinert. 2002. Toward maintaining the genome: DNA damage and replication checkpoints. Annu Rev Genet. 36:617-56.
517. Onate, S. A., Tsai, S. Y., Tsai, M. J., and O'Malley, B. W. 1995. Sequence and characterization of a coactivator for the steroid hormone receptor superfamily. Science 270:1354-1357.
518. Ozanne, D. M., Brady, M. E., Cook, s., Gaughan, L., neal, D. E., and Robson, C. N. 2000. Androgen receptor nuclear translocation is facilitated by the f-actin cross-linking protein filamin. Mol. Endocrinol. 14:1618-1626.
519. Pan H J, Uno H., Inui S., Fulmer N. O., and Chang C. 1999. Roles of testosterone in the growth of keratinocytes through bald frontal dermal papilla cells. Endocrine 11:321-327.
520. Pap, M., and Cooper, G. M. (1998) J Biol Chem 273 (32), 19929-32.
521. Pearce, D. and Yamamoto, K. R. 1993. Mineralocorticoid and glucocorticoid receptor activities distinguished by nonreceptor factors at a composite response element. Science 259:1161-1164.
522. Perrot-Applanat, M., Lescop, P., and Milgrom, E. 1992. The cytoskeleton and the cellular traffic of the progesterone receptor. J. Cell Biol. 119:337-348.
523. Petre, C. E., Wetherill, Y. B., Danielsen, M., and Knudsen, K. E. 2002. Cyclin D1: mechanism and consequence of androgen receptor co-repressor activity. J. Biol. Chem. 277:2207-2215.
524. Pfahl, M. (1993) Endocr Rev 14 (5), 651-8.
525. Quigley, C. A., A. De Bellis, K. B. Marschke, M. K. el-Awady, E. M. Wilson, and F. S. French. 1995. Androgen receptor defects: historical, clinical, and molecular perspectives. Endocr Rev 16:271-321.
526. Rahman, M. M., H. Miyamoto, H. Lardy, and C. Chang. 2003. Inactivation of androgen receptor coregulator ARA55 inhibits androgen receptor activity and agonist effect of antiandrogens in prostate cancer cells. Proc Natl Acad Sci USA 100:5124-9.
527. Rauen, M., M. A. Burtelow, V. M. Dufault, and L. M. Karnitz. 2000. The human checkpoint protein hRad17 interacts with the PCNA-like proteins hRad1, hHus1, and hRadg. J Biol Chem 275:29767-71.
528. Rogatsky, I., Waase, C. L., and Garabedian, M. J. (1998) JBiol Chem 273 (23), 14315-21.
529. Roos-Mattjus, P., K. M. Hopkins, A. J. Oestreich, B. T. Vroman, K. L. Johnson, S. Naylor, H. B. Lieberman, and L. M. Karnitz. 2003. Phosphorylation of human Rad9 is required for genotoxin-activated checkpoint signaling. J Biol Chem 278:24428-37.
530. Sadar, M. D. (1999) JBiol Chem 274 (12), 7777-83.
531. Said, T. K., O. M. Conneely, D. Medina, B. W. O'Malley, and J. P. Lydon. 1997. Progesterone, in addition to estrogen, induces cyclin D1 expression in the murine mammary epithelial cell, in vivo. Endocrinology 138: 3933-9.
532. Santen, R. J. 1992. Clinical review 37: Endocrine treatment of prostate cancer. J. Clin. Endocrinol. Metab. 75:685-689.
533. Sears, R., Nuckolls, F., Haura, E., Taya, Y., Tamai, K., and Nevins, J. R. (2000) Genes Dev 14 (19), 2501-14.
534. Secreto, G. and B. Zumoff. 1994. Abnormal production of androgens in women with breast cancer. Anticancer Res 14:2113-7.
535. Sells, M. A., U. G. Knaus, S. Bagrodia, D. M. Ambrose, G. M. Bokoch, and J. Chemoff. 1997. Human p21-activated kinase (Pak1) regulates actin organization in mammalian cells. Curr Biol 7: 202-10.
536. Shaw, M., Cohen, P., and Alessi, D. R. (1997) FEBSLett 416 (3), 307-11.
537. Siede, W., A. S. Friedberg, and E. C. Friedberg. 1993. RAD9-dependent G1 arrest defines a second checkpoint for damaged DNA in the cell cycle of Saccharomyces cerevisiae. Proc Natl Acad Sci USA 90:7985-9.
538. Silberstein, G. B. 2001. Postnatal mammary gland morphogenesis. Microsc Res Tech 52: 155-62.
539. Simental J. A., Sar M., Lane M. V., French F. S., and Wilson E. M. 1991. Transcriptional activation and nuclear targeting signals of the human androgen receptor. J. Biol. Chem. 266:510-518.
540. St. Onge, R. P., B. D. Besley, J. L. Pelley, and S. Davey. 2003. A role for the phosphorylation of hRad9 in checkpoint signaling. J Biol Chem 278:26620-8.
541. Sugimoto, T., S. Stewart, M. Han, and K. L. Guan. 1998. The kinase suppressor of Ras (KSR) modulates growth factor and Ras signaling by uncoupling Elk-I phosphorylation from MAP kinase activation. Embo J 17:17 17-27.
542. Sui, G., C. Soohoo, B. Affar el, F. Gay, Y. Shi, and W. C. Forrester. 2002. A DNA vector-based RNAi technology to suppress gene expression in mammalian cells. Proc Natl Acad Sci USA 99: 5515-20.
543. Szelei, J., J. Jimenez, A. M. Soto, M. F. Luizzi, and C. Sonnenschein. 1997. Androgen-induced inhibition of proliferation in human breast cancer MCF7 cells transfected with androgen receptor. Endocrinology 138: 1406-12.
544. Thelen, M. P., C. Venclovas, and K. Fidelis. 1999. A sliding clamp model for the Rad1 family of cell cycle checkpoint proteins. Cell 96:769-70.
545. Thin, T. H., L. Wang, E. Kim, L. L. Collins, R. Basavappa, and C. Chang. 2003. Isolation and characterization of androgen receptor mutant, AR (M749L), with hypersensitivity to 17-beta estradiol treatment. J Biol Chem 278:7699-708.
546. Torres, M. A., Eldar-Finkelman, H., Krebs, E. G., and Moon, R. T. (1999) Mol Cell Biol 19 (2), 1427-37.
547. Truica, C. I., Byers, S., and Gelmann, E. P. (2000) Cancer Res 60 (17), 4709-13.
548. Urano, T., T. Saito, T. Tsukui, M. Fujita, T. Hosoi, M. Muramatsu, Y. Ouchi, and S. Inoue. 2002. Efp targets 14-3-3 sigma for proteolysis and promotes breast tumor growth. Nature 417:871-5.
549. Venciovas, C., and M. P. Thelen. 2000. Structure-based predictions of Radl, Radg, Hus 1 and Rad17 participation in sliding clamp and clamp-loading complexes. Nucleic Acids Res 28:2481-93.
550. Volkmer, E., and L. M. Karnitz. 1999. Human homologs of Schizosaccharomyces pombe rad 1, hus 1, and rad9 form a DNA damage-responsive protein complex. J Biol Chem 274:567-70.
551. Wagner, B. L., Norris, J. D., Knotts, T. A., Weigel, N. L., and McDonnell, D. P. 1998. The nuclear corepressors NCoR and SMRT are key regulators of both ligand- and 8-bromo-cyclic AMP-dependent transcriptional activity of the human progesterone receptor. Mol. Cell. Biol. 18: 1369-1378.
552. Wang, Q., Wang, X., Hernandez, A., Hellmich, M. R., Gatalica, Z., and Evers, B. M. (2002) JBiol Chem 24, 24
553. Wang, X., Yang, Y., Guo, X., Sampson, E. R., Hsu, C. L., Tsai, M. Y., Yeh, S., Wu, G., Guo, Y., and Chang, C. (2002) JBiol Chem 277 (18), 15426-31.
554. Watson, P. A., K. Kim, K. S. Chen, and M. N. Gould. 2002. Androgen-dependent mammary carcinogenesis in rats transgenic for the Neu proto-oncogene. Cancer Cell 2: 67-79.
555. Weinert, T. A., and L. H. Hartwell. 1988. The RAD9 gene controls fne ceil cycle response to DNA damage in Saccharomyces cerevisiae. Science 241:317-22.
556. Wen, Y., Hu, M. C., Makino, K., Spohn, B., Bartholomeusz, G., Yan, D. H., and Hung, M. C. (2000) Cancer Res 60 (24), 6841-5.
557. Wilson, C. M. and M. J. McPhaul. 1996. A and B forms of the androgen receptor are expressed in a variety of human tissues. Mol Cell Endocrinol 120:51-7.
558. Woodgett, J. R. (2001) Sci STKE 2001 (100), RE12.
559. Xie, B., S. W. Tsao, and Y. C. Wong. 1999. Sex hormone-induced mammary carcinogenesis in female noble rats: the role of androgens. Carcinogenesis 20: 1597-606.
560. Xu, L., C. K. Glass, and M. G. Rosenfeld. 1999. Coactivator and corepressor complexes in nuclear receptor function. Curr Opin Genet Dev 9:140-7.
561. Yang, L., H. K. Lin, S. Altuwaijri, S. Xie, L. Wang, and C. Chang. 2003. APPL suppresses androgen receptor transactivation via potentiating Akt activity. J Biol Chem 278:16820-7.
562. Yeh, S, and Chang, C. 1996. Cloning and characterization of a specific coactivator, ARA70, for the androgen receptor in human prostate cells. Proc. Natl. Acad. Sci. USA 93: 5517-5521.
563. Yeh, S., Lin, H. K., Kang, H. Y., Thin, T. H., Lin, M. F., and Chang, C. (1999) Proc Natl Acad Sci USA, 96 (10), 5458-63.
564. Yeh, S., M. Y. Tsai, Q. Xu, X. M. Mu, H. Lardy, K. E. Huang, H. Lin, S. D. Yeh, S. Altuwaijri, X. Zhou, L. Xing, B. F. Boyce, M. C. Hung, S. Zhang, L. Gan, and C. Chang. 2002. Generation and characterization of androgen receptor knockout (ARKO) mice: an in vivo model for the study of androgen functions in selective tissues. Proc Natl Acad Sci USA 99:13498-503.
565. Yoshida, K., K. Komatsu, H. G. Wang, and D. Kufe. 2002. c-Ab1 tyrosine kinase regulates the human Rad9 checkpoint protein in response to DNA damage. Mol Cell Biol 22:3292-300.
566. Zheng, P. Eastman, J., Pol, S. V., and Pimplikar, S. W. 1998. PAT1, a microtubule-interacting protein, recognizes the basolateral sorting signal of amyloid precursor protein. Proc. Natl. Acad. Sci. USA 95:14745-14750.
567. Zhou, Z. X., Lane, M. V., Kemppainen, J. A., French, F. S., and Wilson, E. M. 1995. Specificity of ligand-dependent androgen receptor stabilization: receptor domain interactions influence ligand dissociation and receptor stability. Mol. Endocrinol. 9: 208-218. Zou, L., D. Cortez, and S. J. Elledge. 2002. Regulation of ATR substrate selection by Rad17-dependent loading of Rad9 complexes onto chromatin. Genes Dev 16:198-208.

### H. SEQUENCES

1. SEQ ID NO:1 AAH18121. Amyloid beta prec. 585 aa Amyloid beta precursor protein-binding protein 2 [Homo sapiens (ARA67)
2. SEQ ID NO:2 BC018121. Homo sapiens amyl 1758 bp mRNA Homo sapiens amyloid beta precursor protein (cytoplasmic tail) binding protein 2, mRNA complete cds.
3. SEQ ID NO:3 AR protein sequence (Accession No. NM_{—}000044)
4. SEQ ID NO:4 AR cDNA sequence (Accession No. NM_{—}000044)
5. SEQ ID NO:5 GSK3B Protein (Accession No. NP_{—}002084)
6. SEQ ID NO:6 GSK3B DNA (Accession No. NM_{—}002093)
7. SEQ ID NO:7 hRAD9 protein (Accession No. AAB39928)
8. SEQ ID NO:8 hRAD 9 cDNA (Accession No. U53174)
9. SEQ ID NO:9 part of AR siRNA
10. SEQ ID NO:10 Part of AR siRNA
11. SEQ ID NO:11 AR siRNA
12. SEQ ID NO:12 AR siRNA with poly T after U6 promoter
13. SEQ ID NO:13 TR2 protein (Accession No. M21985)
14. SEQ ID NO:14 TR4 protein (Accession No. P49116)
15. SEQ ID NO:15 TR2 cDNA (Accession No. M21985)
16. SEQ ID NO:16 TR4 cDNA (Accession No. P49116)
17. SEQ ID NO:17 Specific primers for hRAD9, (forward)
18. SEQ ID NO:18 Specific primers for hRAD9, (Reverse)
19. SEQ ID NO: 19 18s rRNA primers, (forward)
20. SEQ ID NO: 20 18s rRNA primers, (reverse)
21. SEQ ID NO: 21 Androgen Receptor mutant R614H (AA substitution of R to H at position 608
22. SEQ ID NO:22 Small hRad9 peptide
23. SEQ ID NO:23 Small FXXLL peptide

TABLE 3
ARA67/PAT1 selectively binds to APN in S. cerevisiae^{a}

| | Growth | | | |
|---|---|---|---|---|
| | SD/Glu(-LU) | | SD/Gal(-LU) | |
| Cotransfection | 25° C. | 37° C. | 25° C. | 37° C. |
| pSos + pMyr-ARA67/PAT1 | + | − | + | − |
| pSos-ARN + pMyr-ARA67/PAT1 | + | − | + | + |
| pSos-TR2 + pMyr-ARA67/PAT1 | + | − | + | − |
| pSos-TR4 + pMyr-ARA67/PAT1 | + | − | + | − |
| pSos-ARA55 + pMyr-ARA67/PAT1 | + | − | + | − |
| pSos-APA70 + pMyr-ARA67/PAT1 | + | − | + | − |
| pSos-MAFB + pMyr-ARA67/PAT1 | + | − | + | − |
| pSos-Coll + pMyr-ARA67/PAT1 | + | − | + | − |

^{a}pMyr-ARA67/PAT1 was cotransformed with several other pSos fusion protein constructs. As shown, only ARN interacted with ARA67/PAT1, allowing the yeast host to grow at the stringent temperature of 37° C., while the otter proteins tested could not.

1. A method of screening a subject for breast cancer comprising: a) obtaining a tissue sample, and b) assaying for the presence of androgen receptor, wherein the presence of androgen receptor indicates an increased risk of or presence of breast cancer.

2. The method of claim 1, wherein the screening is in a cell.

3. The method of claim 1, wherein the subject is a mouse.

4. The method of claim 1, wherein the subject is a human.

5. The method of claim 1, wherein the subject is male.

6. A method of screening a subject for breast cancer comprising: a) obtaining a tissue sample, and b) assaying for the presence of androgen receptor mRNA, wherein the presence of androgen receptor indicates an increased risk of or presence of breast cancer.

7. The method of claim 6, wherein the screening is in a cell.

8. The method of claim 6, wherein the subject is a mouse.

9. The method of claim 6, wherein the subject is a human.

10. The method of claim 6, wherein the subject is male.

11. A method of treating cancer comprising administering to a subject an androgen receptor inhibitor.

12. The method of claim 11, wherein the androgen receptor inhibitor reduces nuclear translocation of androgen receptor.

13. The method of claim 12, wherein the androgen receptor inhibitor comprises ARA67, or fragment thereof.

14. The method of claim 11, wherein the androgen receptor inhibitor phosphorylates androgen receptor.

15. The method of claim 14, wherein the androgen receptor inhibitor comprises GSK2B or fragment thereof.

16. The method of claim 11, wherein the androgen receptor inhibitor reduces an interaction between the N-terminus and C terminus of androgen receptor.

17. The method of claim 16, wherein the androgen receptor inhibitor comprises hRad9 or fragment thereof.

18. The method of claim 11, wherein the androgen receptor inhibitor is ARA67, GSK2B, or hRad9, or fragment thereof.

19. The method of claim 11, wherein the androgen receptor inhibitor interacts with androgen receptor mRNA.

20. The method of claim 19, wherein the androgen receptor inhibitor comprises a functional nucleic acid.

21. The method of claim 20, wherein the androgen receptor inhibitor comprises an siRNA.

22. The method of claim 21, wherein the siRNA comprises SEQ ID NO:11.

23. The method of claim 11, wherein the cancer is breast cancer.

24. The method of claim 11, wherein the subject is a male.

25. A method of screening a composition for the ability to modulate AR activity comprising administering the compound to a system, wherein the system comprises AR and ARA67, GSK2B, or hRad9, and determining if the compound reduces the interaction between AR and ARA67, GSK2B, or hRad9.

26. A method of screening a composition for the ability to modulate AR activity comprising administering the compound to a system, wherein the system comprises AR and determining if the compound decreases the amount of nuclear AR.

27. A method of screening a composition for the ability to modulate AR activity comprising administering the compound to a system, wherein the system comprises AR and determining if the compound decreases the amount of phoshorylated AR.

28. A method of screening a composition for the ability to modulate AR activity comprising administering the compound to a system, wherein the system comprises AR and determining if the compound decreases the amount of N-terminus AR interacting with the C-terminus of AR.

29. The method of claim 28, wherein the system is a breast cancer cell or cell line.

30. The method of claim 29, wherein the breast cancer cell line is MCF-7, 7R-75-1, or T47-D.

31. A composition for inhibiting androgen receptor activity comprising a protein, peptide, antibody, or functional nucleic acid, wherein the composition reduces AR translocation to the nucleus, wherein the composition is not SEQ ID NO:1.

32. The composition of claim 31, wherein the composition comprises a fragment of ARA67, wherein the fragment binds androgen receptor.

33. A composition for inhibiting androgen receptor activity comprising a protein, peptide, antibody, or functional nucleic acid, wherein the composition reduces the interaction between the AR N-terminus and the AR C-terminus, wherein the composition is not SEQ ID NO:7.

34. The composition of claim 33, wherein the composition comprises a fragment of hRad9, wherein the fragment binds androgen receptor.

35. A composition for inhibiting androgen receptor activity comprising a functional nucleic acid, wherein the functional nucleic acid interacts with the mRNA of AR.

36. The composition of claim 35, wherein the composition comprises an siRNA.

37. The composition of claim 36, wherein the siRNA comprises SEQ ID NO:11.

38. A composition for inhibiting androgen receptor activity comprising an antibody or a functional nucleic acid, wherein the composition reduces AR translocation to the nucleus, and wherein the composition competes with ARA67 for binding to androgen receptor, wherein the composition is not SEQ ID NO:1.

39. A composition for inhibiting androgen receptor activity comprising an antibody or a functional nucleic acid, wherein the composition reduces AR translocation to the nucleus, and wherein the composition competes with hRad9 for binding to androgen receptor, wherein the composition is not SEQ ID NO:7.

40. A composition for inhibiting androgen receptor activity comprising an antibody or a functional nucleic acid, wherein the composition reduces AR translocation to the nucleus, and wherein the composition competes with GSK2B for binding to androgen receptor wherein the composition is not SEQ ID NO:5.

41. A composition for inhibiting androgen receptor activity comprising an antibody or a functional nucleic acid, wherein the composition reduces AR translocation to the nucleus, and wherein the composition binds androgen receptor as ARA67 binds androgen receptor, wherein the composition is not SEQ ID NO:1.

42. A composition for inhibiting androgen receptor activity comprising an antibody or a functional nucleic acid, wherein the composition reduces AR translocation to the nucleus, and wherein the composition binds androgen receptor as hRad9 binds androgen receptor wherein the composition is not SEQ ID NO:7.

43. A composition for inhibiting androgen receptor activity comprising an antibody or a functional nucleic acid, wherein the composition reduces AR translocation to the nucleus, and wherein the composition binds androgen receptor as binds androgen receptor wherein the composition is not SEQ ID NO:5.

44. The composition of claim 38, wherein the composition is an antibody.

45. The composition of claim 44, wherein the antibody is a monoclonal antibody.

46. The composition of claim 44, wherein the antibody is a polyclonal antibody.

47. The composition of claim 38, wherein the composition is a functional nucleic acid.

48. The composition of claim 47, wherein the functional nucleic acid is an aptamer.

49. A compound produced by the method of screening a compound for the ability to modulate AR activity comprising administering the compound to a system, wherein the system comprises AR and ARA67, GSK2B, or hRad9, and determining if the compound reduces the interaction between AR and ARA67, GSK2B, or hRad9 and making the compound.

50. A compound produced by the method of screening a compound for the ability to modulate AR activity comprising administering the compound to a system, wherein the system comprises AR and determining if the compound decreases the amount of nuclear AR and making the compound.

51. A compound produced by the method of screening a compound for the ability to modulate AR activity comprising administering the compound to a system, wherein the system comprises AR and determining if the compound decreases the amount of phoshorylated AR and making the compound.

52. A compound produced by the method of screening a compound for the ability to modulate AR activity comprising administering the compound to a system, wherein the system comprises AR and determining if the compound decreases the amount of N-terminus Ar interacting with the C-terminus of AR and making the compound.
